(19) United States
(12) Patent Application Publication
(10) Pub. No.: US 2010/0105058 A1
(43) Pub. Date: Apr. 29, 2010

(54) Method for Measuring DNA Methylation

(75) Inventors: Yoshitaka Tomigahara, Osaka (JP); Hirokazu Tarui, Osaka (JP)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: SUMITOMO CHEMICAL COMPANY, LIMITED, Chuo-ku, Tokyo (JP)

(21) Appl. No.: 12/593,100

(22) PCT Filed: Mar. 26, 2008
(86) PCT No.: PCT/JP2008/056525

§ 371 Date: Sep. 25, 2009

(30) Foreign Application Priority Data
Mar. 26, 2007 (JP) 2007-078661

(51) Int. Cl. C12Q 1/68

(52) U.S. Cl. 435/6

(57) ABSTRACT

The present invention relates to a method of measuring the content of methylated DNA in a DNA region of interest in a genomic DNA contained in a biological specimen, and so on.

### TECHNICAL FIELD

The present invention relates to a method of measuring the content of methylated DNA in a DNA region of interest in a genomic DNA contained in a biological specimen, and so on.

### BACKGROUND ART

As a method for evaluating the methylation state of DNA in an objective DNA region in a genomic DNA contained in a biological specimen, for example, there is known a method of measuring the content of methylated DNA in an objective DNA region in a genomic DNA (see, for example, Nucleic Acids Res., 1994 Aug. 11; 22(15): 2990-7, and Proc. Natl. Acad. Sci. U.S.A., 1997 Mar. 18; 94(6): 2284-9 for reference). In such a measuring method, first, it is necessary to extract DNA containing the objective DNA region from a DNA sample derived from a genomic DNA, and the extracting operation is complicated.

As a method of measuring the content of methylated DNA in an objective region of extracted DNA, for example, (1) a method of amplifying an objective region by subjecting the DNA to a chain reaction for DNA synthesis by DNA polymerase after modification of the DNA with a sulfite or the like (Polymerase Chain Reaction; hereinafter also referred to as PCR), and (2) a method of amplifying an objective region by subjecting the DNA to PCR after digestion of the DNA using a methylation sensitive restriction enzyme are known. Both of these methods require time and labor for DNA modification for detection of methylation, subsequent purification of the product, preparation of a reaction system for PCR, and checking of DNA amplification.

That is, the present invention provides:

1. A method of measuring the content of methylated DNA in a target DNA region in genomic DNA contained in a biological specimen, comprising:
(1) First step of selecting methylated single-stranded DNA, by First (A) step of separating methylated single-stranded DNA from a DNA sample derived from genomic DNA contained in a biological specimen, and First (B) step of causing binding between the single-stranded DNA separated in First (A) step and an immobilized methylated DNA antibody,
(2) Second step of mixing the single-stranded DNA selected in First step with a masking oligonucleotide comprising a nucleotide sequence complementary to a nucleotide sequence of a recognition site of a methylation sensitive restriction enzyme and digesting the selected single-stranded DNA with at least one kind of methylation sensitive restriction enzyme, and
(3) Third step comprising as pre steps of the following regular steps:

Step (Third (pre A) step) of separating single-stranded DNA (single-stranded DNA not containing an unmethylated CpG in a recognition site of a methylation sensitive restriction enzyme protected by the masking oligonucleotide) which is an undigested substance obtained in Second step from the immobilized methylated DNA antibody and the masking oligonucleotide into DNA in a single-stranded state (plus strand);

Step (Third (pre B) step) of extensionally forming double-stranded DNA from single-stranded DNA (plus strand) containing the target DNA region by extending DNA (plus strand) derived from a genome that is made into a single-stranded state in Third (pre A) step by one extension of an extension primer using the extension primer (forward primer) comprising a nucleotide sequence (minus strand) which is complementary to a partial nucleotide sequence (plus strand) of the nucleotide sequence comprised by the single-stranded DNA (plus strand), the partial nucleotide sequence (plus strand) located on further 3′-end side than 3′-end of the nucleotide sequence (plus strand) of the target DNA region, as the extension primer; and

Step (Third (pre C) step) of temporarily separating the double-stranded DNA extensionally formed in Third (pre B) step into single-stranded DNA (plus strand) containing the target DNA region and single-stranded DNA (minus strand) containing the target DNA region, and as regular steps:

(a) Third step-A of extensionally forming double-stranded DNA from single-stranded DNA containing the target DNA region by one extension of an extension primer by using the generated single-stranded DNA (plus strand) containing the target DNA region as a template, and the forward primer as an extension primer, and

(b) Third step-B of extensionally forming double-stranded DNA from single-stranded DNA containing the target DNA region by one extension of an extension primer by using the generated single-stranded DNA (minus strand) containing the target DNA region as a template, and an extension primer (reverse primer) comprising a nucleotide sequence (plus strand) which is complementary to a partial nucleotide sequence (minus strand) of the nucleotide sequence comprised by the single-stranded DNA (minus strand) containing the target DNA region, the partial nucleotide sequence (minus strand) located on further 3′-end side than 3′-end of the nucleotide sequence (minus strand) complementary to the nucleotide sequence (plus strand) of the target DNA region as the extension primer,

wherein by repeating the regular steps after temporarily separating the extensionally formed double-stranded DNA obtained in each of the regular steps into a single-stranded state, the methylated DNA in the target DNA region is amplified to a detectable level and a content of the amplified DNA is quantified;

2. The method according to the above 1, wherein the methylated DNA antibody in Third step is a methyl cytosine antibody;
3. The method according to either of the above 1 to 2, wherein the biological specimen is serum or plasma of a mammal;
4. The method according to either of the above 1 to 2, wherein the biological specimen is blood or a bodily fluid of a mammal;
5. The method according to either of the above 1 to 2, wherein the biological specimen is a cell lysate or a tissue lysate;
6. The method according to any one of the above 1 to 5, wherein the DNA sample derived from genomic DNA contained in a biological specimen is a DNA sample preliminarily digested with a restriction enzyme whose recognition cleaving site excludes the target DNA region comprised by the genomic DNA;
7. The method according to any one of the above 1 to 6, wherein the DNA sample derived from genomic DNA contained in a biological specimen is a DNA sample digested with at least one kind of methylation sensitive restriction enzyme;
8. The method according to any one of the above 1 to 7, wherein the DNA sample derived from genomic DNA contained in a biological specimen is a preliminarily purified DNA sample;
9. The method according to any one of the above 1 to 8, wherein the at least one kind of methylation sensitive restriction enzyme is a restriction enzyme having a recognition cleaving site in the target DNA region comprised by the genomic DNA contained in the biological specimen;
10. The method according to any one of the above 1 to 9, wherein the at least one kind of methylation sensitive restriction enzyme is HpaII or HhaI which is a methylation sensitive restriction enzyme;
11. The method according to any one of the above 1 to 10, wherein Third step is executed without conducting the digesting with the methylation sensitive restriction enzyme in Second step; and
12. The method according to any one of the above 1 to 11, wherein a counter oligonucleotide is added when methylated single-stranded DNA is separated in First (A) step.

### BRIEF DESCRIPTION OF THE DRAWINGS

FIG. 1 shows results of 1.5% agarose gel electrophoresis of amplification products obtained by amplifying methylated DNA in the region comprising the nucleotide sequence of SEQ ID NO: 23 by PCR from a prepared sample in Example 1.

From the leftmost lane in the drawing, results in a DNA marker “MK”, a sample “M” of a solution of a partially methylated oligonucleotide GPR7-2079-2176/98 mer-M(7) in which the recognition sequence of HpaII is methylated, subjected to an “A” treatment, a sample “H” of a solution of a partially methylated oligonucleotide GPR7-2079-2176/98 mer-HM(5) in which part of the recognition sequence of HpaII is not methylated, subjected to an “A” treatment, a sample “U” of a solution of an unmethylated oligonucleotide GPR7-2079-2176/98 mer-UM, subjected to an “A” treatment, a sample “M” of a solution of a partially methylated oligonucleotide GPR7-2079-2176/98 mer-M(7) in which the recognition sequence of HpaII is methylated, subjected to a “B” treatment, a sample “H” of a solution of a partially methylated oligonucleotide GPR7-2079-2176/98 mer-HM(5) in which part of the recognition sequence of HpaII is not methylated, subjected to a “B” treatment, a sample “U” of a solution of an unmethylated oligonucleotide GPR7-2079-2176/98 mer-UM, subjected to a “B” treatment, a sample “m” of a solution of a partially methylated oligonucleotide GPR7-2079-2176/98 mer-M(7) in which the recognition sequence of HpaII is methylated, subjected to a “C” treatment, a sample “H” of a solution of a partially methylated oligonucleotide GPR7-2079-2176/98 mer-HM(5) in which part of the recognition sequence of HpaII is not methylated, subjected to a “C” treatment, and a sample “U” of a solution of an unmethylated oligonucleotide GPR7-2079-2176/98 mer-UM, subjected to a “C” treatment are shown.

FIG. 2 shows results of 1.5% agarose gel electrophoresis of amplification products obtained by amplifying methylated DNA in the target DNA region comprising the nucleotide sequence of SEQ ID NO: 28 by PCR from a prepared sample in Example 2. From the leftmost lane in the drawing, results in a DNA marker “MK”, a solution “MD” of a methylated DNA fragment MX (negative control), a solution “D” of an unmethylated DNA fragment X (negative control), a solution “MC” of a methylated DNA fragment MX, a solution “C” of an unmethylated DNA fragment X, a solution “MB” of a methylated DNA fragment MX, a solution “B” of an unmethylated DNA fragment X, a solution “MA” of a methylated DNA fragment MX, and a solution “A” of an unmethylated DNA fragment X are shown.

FIG. 3 shows results of 1.5% agarose gel electrophoresis of amplification products obtained by amplifying methylated DNA in the target DNA region comprising the nucleotide sequence of SEQ ID NO: 46 by PCR from a prepared sample in Example 3. From the leftmost lane in the drawing, results in a DNA marker “MK”, a solution “MD” of a methylated DNA fragment MY (negative control), a solution “D” of an unmethylated DNA fragment Y (negative control), a solution “MC” of a methylated DNA fragment MY, a solution “C” of an unmethylated DNA fragment X, a solution “MB” of a methylated DNA fragment MY, a solution “B” of an unmethylated DNA fragment Y, a solution “MA” of a methylated DNA fragment MA, and a solution “A” of an unmethylated DNA fragment Y are shown.

FIG. 4 shows results of 1.5% agarose gel electrophoresis of amplification products obtained by amplifying methylated DNA in the target DNA region comprising the nucleotide sequence of SEQ ID NO: 55 by PCR from a prepared sample in Example 4. From the leftmost lane in the drawing, results in a DNA marker “MK”, a solution “MC” of a methylated DNA fragment MZ (negative control), a solution “C” of an unmethylated DNA fragment Z (negative control), a solution “MB” of a methylated DNA fragment MZ, a solution “B” of an unmethylated DNA fragment Z, a solution “MA” of a methylated DNA fragment MZ, a solution “B” of an unmethylated DNA fragment Z, a solution “MA” of a methylated DNA fragment MZ, and a solution “A” of an unmethylated DNA fragment Z are shown.

FIG. 5 shows results of 1.5% agarose gel electrophoresis of amplification products obtained by amplifying methylated DNA in the target DNA region comprising the nucleotide sequence of SEQ ID NO: 61 by PCR from a prepared sample in Example 5. From the leftmost lane in the drawing, results in a DNA marker “MK”, a solution “MD” of a methylated genomic DNA (negative control), a solution “D” of an unmethylated genomic DNA (negative control), a solution “MC” of a methylated genomic DNA, a solution “C” of an unmethylated genomic DNA, a solution “MB” of a methylated genomic DNA, a solution “B” of an unmethylated genomic DNA, a solution “MA” of a methylated genomic DNA, and a solution “A” of an unmethylated genomic DNA are shown.

### BEST MODE FOR CARRYING OUT THE INVENTION

As the “biological specimen” in the present invention, for example, a cell lysate, a tissue lysate (here the term “tissue” is used in a broad sense including blood, lymph node and so on) or biological samples including bodily sections such as plasma, serum and lymph, bodily secretions (urine, milk and so on) and the like and a genomic DNA obtained by extracting these biological samples, in mammals can be recited. As a biological specimen, for example, samples derived from microorganisms, viruses and the like can be recited, and in such a case, “a genomic DNA” in the present measuring method also means genomic DNA of microorganisms, viruses and the like.

When the specimen derived from a mammal is blood, use of the present measuring method in a regular health check or a simple examination is expected.

For obtaining a genomic DNA from a specimen derived from a mammal, for example, DNA may be extracted using a commercially available DNA extraction kit.

When blood is used as a specimen, plasma or serum is prepared from blood in accordance with a commonly used method, and using the prepared plasma or serum as a specimen, free DNA (including DNA derived from cancer cells such as gastric cancer cells) contained in the specimen is analyzed. This enables analysis of DNA derived from cancer cells such as gastric cancer cells while avoiding DNA derived from hemocytes, and improves the sensitivity of detection of cancer cells such as gastric cancer cells and a tissue containing the same.

Usually, a gene (a genomic DNA) consists of four kinds of bases. In these bases, such a phenomenon is known that only cytosine is methylated, and such methylation modification of DNA is limited to cytosine in a nucleotide sequence represented by 5′-CG-3′ (C represents cytosine, and G represents guanine. Hereinafter, the nucleotide sequence is also referred to as “CpG”). The site to be methylated in cytosine is its position 5. In DNA replication prior to cell division, only cytosine in “CpG” of a template chain is methylated immediately after replication, however, cytosine in “CpG” of a newly-generated strand is immediately methylated by the action of methyltransferase. Therefore, the methylation state of DNA will be passed to new two sets of DNA even after DNA replication. The term “methylated DNA” in the present invention means DNA occurring by such methylation modification.

The term “CpG pair” in the present invention means double-stranded oligonucleotide in which a nucleotide sequence represented by CpG and a CpG that is complement with this are base-paired.

The term “objective DNA region” (hereinafter, also referred to as an “objective region”) used in the present invention means a DNA region for which presence or absence of methylation of cytosine included in the region is to be examined, and has a recognition site of at least one kind of methylation sensitive restriction enzyme. A DNA region containing at least one cytosine in a nucleotide sequence represented by CpG which is present in a nucleotide sequence of a promoter region, an untranslated region, or a translated region (coding region) of a useful protein gene such as Lysyl oxidase, HRAS-like suppressor, bA305P22.2.1, Gamma filamin, HAND1, Homologue of RIKEN 2210016F16, FLJ32130, PPARG angiopoietin-related protein, Thrombomodulin, p53-responsive gene 2, Fibrillin2, Neurofilament3, disintegrin and metalloproteinase domain 23, G protein-coupled receptor 7, G-protein coupled somatostatin and angiotensin-like peptide receptor, Solute carrier family 6 neurotransmitter transporter noradrenalin member 2 and so on can be recited.

To be more specific, when the useful protein gene is a Lysyl oxidase gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Lysyl oxidase gene derived from human, and a promoter region located 5′ upstream of the same can be recited, and more concretely, the nucleotide sequence of SEQ ID NO: 1 (corresponding to a nucleotide sequence represented by base No. 16001 to 18661 in the nucleotide sequence described in Genbank Accession No. AF270645) can be recited. In the nucleotide sequence of SEQ ID NO: 1, ATG codon encoding methionine at amino terminal of Lysyl oxidase protein derived from human is represented in base No. 2031 to 2033, and a nucleotide sequence of the above exon 1 is represented in base No. 1957 to 2661. Cytosine in the nucleotide sequence represented by CpG which is present in the nucleotide sequence of SEQ ID NO: 1, in particular, cytosine in CpG which is present in a region where CpGs are densely present in the nucleotide sequence of SEQ ID NO: 1 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1539, 1560, 1574, 1600, 1623, 1635, 1644, 1654, 1661, 1682, 1686, 1696, 1717, 1767, 1774, 1783, 1785, 1787, 1795 and so on in the nucleotide sequence of SEQ ID NO: 1 can be recited.

To be more specific, when the useful protein gene is a HRAS-like suppressor gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a HRAS-like suppressor gene derived from human, and a promoter region located 5′ upstream of the same can be recited, and more concretely, the nucleotide sequence of SEQ ID NO: 2 (corresponding to a nucleotide sequence represented by base No. 172001 to 173953 in the nucleotide sequence described in Genbank Accession No. AC068162) can be recited. In the nucleotide sequence of SEQ ID NO: 2, the nucleotide sequence of exon 1 of a HRAS-like suppressor gene derived from human is represented in base No. 1743 to 1953. Cytosine in the nucleotide sequence represented by CpG which is present in the nucleotide sequence of SEQ ID NO: 2, in particular, cytosine in CpG which is present in a region where CpGs are densely present in the nucleotide sequence of SEQ ID NO; 2 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1316, 1341, 1357, 1359, 1362, 1374, 1390, 1399, 1405, 1409, 1414, 1416, 1422, 1428, 1434, 1449, 1451, 1454, 1463, 1469, 1477, 1479, 1483, 1488, 1492, 1494, 1496, 1498, 1504, 1510, 1513, 1518, 1520 and so on in the nucleotide sequence of SEQ ID NO: 2 can be recited.

To be more specific, when the useful protein gene is a bA305P22.2.1 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a bA305P22.2.1 gene derived from human, and a promoter region located 5′ upstream of the same can be recited, and more concretely, the nucleotide sequence of SEQ ID NO: 3 (corresponding to a nucleotide sequence represented by base No. 13001 to 13889 in the nucleotide sequence described in Genbank Accession No. AL121673) can be recited. In the nucleotide sequence of SEQ ID NO: 3, ATG codon encoding methionine at amino terminal of bA305P22.2.1 protein derived from human is represented in base No. 849 to 851, and a nucleotide sequence of the above exon 1 is represented in base No. 663 to 889. Cytosine in the nucleotide sequence represented by CpG which is present in the nucleotide sequence of SEQ ID NO: 3, in particular, cytosine in CpG which is present in a region where CpGs are densely present in the nucleotide sequence of SEQ ID NO: 3 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 329, 335, 337, 351, 363, 373, 405, 424, 427, 446, 465, 472, 466 and so on in the nucleotide sequence of SEQ ID NO: 3 can be recited.

To be more specific, when the useful protein gene is a Gamma filamin gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Gamma filamin gene derived from human, and a promoter region located 5′ upstream of the same can be recited, and more concretely, the nucleotide sequence of SEQ ID NO: 4 (corresponding to a complementary sequence to a nucleotide sequence represented by base No. 63528 to 64390 in the nucleotide sequence described in Genbank Accession No. AC074373) can be recited. In the nucleotide sequence of SEQ ID NO: 4, ATG codon encoding methionine at amino terminal of Gamma filamin protein derived from human is represented in base No. 572 to 574, and a nucleotide sequence of the above exon 1 is represented in base No. 463 to 863. Cytosine in the nucleotide sequence represented by CpG which is present in the nucleotide sequence of SEQ ID NO: 4, in particular, cytosine in CpG which is present in a region where CpGs are densely present in the nucleotide sequence of SEQ ID NO: 4 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 329, 333, 337, 350, 353, 360, 363, 370, 379, 382, 384, 409, 414, 419, 426, 432, 434, 445, 449, 459, 472, 474, 486, 490, 503, 505 and so on in the nucleotide sequence of SEQ ID NO; 4 can be recited.

To be more specific, when the useful protein gene is a HAND1 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a HAND1 gene derived from human, and a promoter region located 5′ upstream of the same can be recited, and more concretely, the nucleotide sequence of SEQ ID NO: 5 (corresponding to a complementary sequence to a nucleotide sequence represented by base No. 24303 to 26500 in the nucleotide sequence described in Genbank Accession No. AC026688) can be recited. In the nucleotide sequence of SEQ ID NO: 5, ATG codon encoding methionine at amino terminal of HAND1 protein derived from human is represented in base No. 1656 to 1658, and a nucleotide sequence of the above exon 1 is represented in base No. 1400 to 2198. Cytosine in the nucleotide sequence represented by CpG which is present in the nucleotide sequence of SEQ ID NO: 5, in particular, cytosine in CpG which is present in a region where CpGs are densely present in the nucleotide sequence of SEQ ID NO: 5 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1153, 1160, 1178, 1187, 1193, 1218, 1232, 1266, 1272, 1292, 1305, 1307, 1316, 1356, 1377, 1399, 1401, 1422, 1434 and so on in the nucleotide sequence of SEQ ID NO: 5 can be recited.

To be more specific, when the useful protein gene is a Homologue of RIKEN 2210016F16 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Homologue of RIKEN 2210016F16 gene derived from human, and a promoter region located 5′ upstream of the same can be recited, and more concretely, the nucleotide sequence of SEQ ID NO: 6 (corresponding to a complementary nucleotide sequence to a nucleotide sequence represented by base No. 157056 to 159000 in the nucleotide sequence described in Genbank Accession No. AL354733) can be recited. In the nucleotide sequence of SEQ ID NO: 6, a nucleotide sequence of exon 1 of a Homologue of a RIKEN 2210016F16 gene derived from human is represented in base No. 1392 to 1945. Cytosine in the nucleotide sequence represented by CpG which is present in the nucleotide sequence of SEQ ID NO: 6, in particular, cytosine in CpG which is present in a region where CpGs are densely present in the nucleotide sequence of SEQ ID NO: 6 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1172, 1175, 1180, 1183, 1189, 1204, 1209, 1267, 1271, 1278, 1281, 1313, 1319, 1332, 1334, 1338, 1346, 1352, 1358, 1366, 1378, 1392, 1402, 1433, 1436, 1438 and so on in the nucleotide sequence of SEQ ID NO: 6 can be recited.

To be more specific, when the useful protein gene is a FLJ32130 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a FLJ32130 gene derived from human, and a promoter region located 5′ upstream of the same can be recited, and more concretely, the nucleotide sequence of SEQ ID NO: 7 (corresponding to a complementary nucleotide sequence to a nucleotide sequence represented by base No. 1 to 2379 in the nucleotide sequence described in Genbank Accession No. AC002310) can be recited. In the nucleotide sequence of SEQ ID NO: 7, ATG codon encoding methionine at amino terminal of FLJ32130 protein derived from human is represented in base No. 2136 to 213B, and a nucleotide sequence assumed to be the above exon 1 is represented in base No. 2136 to 2379. Cytosine in the nucleotide sequence represented by CpG which is present in the nucleotide sequence of SEQ ID NO: 7, in particular, cytosine in CpG which is present in a region where CpGs are densely present in the nucleotide sequence of SEQ ID NO: 7 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1714, 1716, 1749, 1753, 1762, 1795, 1814, 1894, 1911, 1915, 1925, 1940, 1955, 1968 and so on in the nucleotide sequence of SEQ ID NO: 7 can be recited.

To be more specific, when the useful protein gene is a PPARG angiopoietin-related protein gene, as a nucleotide sequence that includes at least one nucleotide sequence represented by CpG present in a nucleotide sequence of its promoter region, untranslated region or translated region (coding region), a nucleotide sequence or a genomic DNA containing axon 1 of a PPARG angiopoietin-related protein gene derived from human, and a promoter region located 5′ upstream of the same can be recited, and more concretely, the nucleotide sequence of SEQ ID NO: 8 can be recited. In the nucleotide sequence of SEQ ID NO: 8, ATG codon encoding methionine at amino terminal of PPARG angiopoietin-related protein derived from human is represented in base No. 717 to 719, and a nucleotide sequence of the 5′ side part of the above exon 1 is represented in base No. 1957 to 2661. Cytosine in the nucleotide sequence represented by CpG which is present in the nucleotide sequence of SEQ ID NO: 8, in particular, cytosine in CpG which is present in a region where CpGs are densely present in the nucleotide sequence of SEQ ID NO: 8 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 35, 43, 51, 54, 75, 85, 107, 127, 129, 143, 184, 194, 223, 227, 236, 251, 258 and so on in the nucleotide sequence of SEQ ID NO: 8 can be recited.

To be more specific, when the useful protein gene is a Thrombomodulin gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Thrombomodulin gene derived from human, and a promoter region located 5′ upstream of the same can be recited, and more concretely, the nucleotide sequence of SEQ ID NO: 9 (corresponding to a nucleotide sequence represented by base No. 1 to 6096 in the nucleotide sequence described in Genbank Accession No. AF495471) can be recited. In the nucleotide sequence of SEQ ID NO: 9, ATG codon encoding methionine at amino terminal of Thrombomodulin protein derived from human is represented in base No. 2590 to 2592, and a nucleotide sequence of the above exon 1 is represented in base No. 2048 to 6096. Cytosine in the nucleotide sequence represented by CpG which is present in the nucleotide sequence of SEQ ID NO: 9, in particular, cytosine in CpG which is present in a region where CpGs are densely present in the nucleotide sequence of SEQ ID NO: 9 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1539, 1551, 1571, 1579, 1581, 1585, 1595, 1598, 1601, 1621, 1632, 1638, 1645, 1648, 1665, 1667, 1680, 1698, 1710, 1724, 1726, 1756 and so on in the nucleotide sequence of SEQ ID NO: 9 can be recited.

To be more specific, when the useful protein gene is a p53-responsive gene 2 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a p53-responsive gene 2 gene derived from human, and a promoter region located 5′ upstream of the same can be recited, and more concretely, the nucleotide sequence of SEQ ID NO: 10 (corresponding to a complementary sequence to a nucleotide sequence represented by base No. 113501 to 116000 in the nucleotide sequence described in Genbank Accession No. AC009471) can be recited. In the nucleotide sequence of SEQ ID NO: 10, a nucleotide sequence of exon 1 of a p53-responsive gene 2 gene derived from human is represented in base No. 1558 to 1808. Cytosine in the nucleotide sequence represented by CpG which is present in the nucleotide sequence of SEQ ID NO: 10 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 1282, 1284, 1301, 1308, 1315, 1319, 1349, 1351, 1357, 1361, 1365, 137B, 1383 and so on in the nucleotide sequence of SEQ ID NO: 10 can be recited.

To be more specific, when the useful protein gene is a Fibrillin2 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Fibrillin2 gene derived from human, and a promoter region located 5′ upstream of the same can be recited, and more concretely, the nucleotide sequence of SEQ ID NO: 11 (corresponding to a complementary sequence to a nucleotide sequence represented by base No. 118801 to 121000 in the nucleotide sequence described in Genbank Accession No. AC113387) can be recited. In the nucleotide sequence of SEQ ID NO: 11, a nucleotide sequence of exon 1 of a Fibrillin2 gene derived from human is represented in base No. 1091 to 1345. Cytosine in the nucleotide sequence represented by CpG which is present in the nucleotide sequence of SEQ ID NO: 11 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 679, 687, 690, 699, 746, 773, 777, 783, 795, 799, 812, 823, 830, 834, 843 and so on in the nucleotide sequence of SEQ ID NO: 11 can be recited.

To be more specific, when the useful protein gene is a Neurofilament3 gene, as a nucleotide sequence that includes at least one nucleotide sequence represented by CpG present in a nucleotide sequence of its promoter region, untranslated region or translated region (coding region), a nucleotide sequence of a genomic DNA containing axon 1 of a Neurofilament3 gene derived from human, and a promoter region located 5′ upstream of the same can be recited, and more concretely, the nucleotide sequence of SEQ ID NO: 12 (corresponding to a complementary sequence to a nucleotide sequence represented by base No. 28001 to 30000 in the nucleotide sequence described in Genbank Accession No. AF106564) can be recited. In the nucleotide sequence of SEQ ID NO: 12, a nucleotide sequence of exon 1 of a Neurofilament3 gene derived from human is represented in base No. 614 to 1694. Cytosine in the nucleotide sequence represented by CpG which is present in the nucleotide sequence of SEQ ID NO: 12 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 428, 432, 443, 451, 471, 475, 482, 491, 499, 503, 506, 514, 519, 532, 541, 544, 546, 563, 566, 572, 580 and so on in the nucleotide sequence of SEQ ID NO: 12 can be recited.

To be more specific, when the useful protein gene is a disintegrin and metalloproteinase domain 23 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a disintegrin and metalloproteinase domain 23 gene derived from human, and a promoter region located 5′ upstream of the same can be recited, and more concretely, the nucleotide sequence of SEQ ID NO: 13 (corresponding to a nucleotide sequence represented by base No. 21001 to 23300 in the nucleotide sequence described in Genbank Accession No. AC009225) can be recited. In the nucleotide sequence of SEQ ID NO: 13, a nucleotide sequence of exon 1 of a disintegrin and metalloproteinase domain 23 gene derived from human is represented in base No. 1194 to 1630. Cytosine in the nucleotide sequence represented by CpG which is present in the nucleotide sequence of SEQ ID NO: 13 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 998, 1003, 1007, 1011, 1016, 1018, 1020, 1026, 1028, 1031, 1035, 1041, 1043, 1045, 1051, 1053, 1056, 1060, 1066, 1068, 1070, 1073, 1093, 1096, 1106, 1112, 1120, 1124, 1126 and so on in the nucleotide sequence of SEQ ID NO: 13 can be recited.

To be more specific, when the useful protein gene is a G protein-coupled receptor 7 gene, as a nucleotide sequence that includes at least one nucleotide sequence represented by CpG present in a nucleotide sequence of its promoter region, untranslated region or translated region (coding region), a nucleotide sequence of a genomic DNA containing axon 1 of a G protein-coupled receptor 7 gene derived from human, and a promoter region located 5′ upstream of the same can be recited, and more concretely, the nucleotide sequence of SEQ ID NO: 14 (corresponding to a nucleotide sequence represented by base No. 75001 to 78000 in the nucleotide sequence described in Genbank Accession No. AC009800) can be recited. In the nucleotide sequence of SEQ ID NO: 14, a nucleotide sequence of axon 1 of a G protein-coupled receptor 7 gene derived from human is represented in base No. 1666 to 2652. Cytosine in the nucleotide sequence represented by CpG which is present in the nucleotide sequence of SEQ ID NO: 14 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 1480, 1482, 1485, 1496, 1513, 1526, 1542, 1560, 1564, 1568, 1570, 1580, 1590, 1603, 1613, 1620 and so on in the nucleotide sequence of SEQ ID NO: 14 can be recited.

To be more specific, when the useful protein gene is a G-protein coupled somatostatin and angiotensin-like peptide receptor gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a G-protein coupled somatostatin and angiotensin-like peptide receptor gene derived from human, and a promoter region located 5′ upstream of the same can be recited, and more concretely, the nucleotide sequence of SEQ ID NO: 15 (corresponding to a complementary sequence to a nucleotide sequence represented by base No. 57001 to 60000 in the nucleotide sequence described in Genbank Accession No. AC008971) can be recited. In the nucleotide sequence of SEQ ID NO: 15, a nucleotide sequence of exon 1 of a G-protein coupled somatostatin and angiotensin-like peptide receptor gene derived from human is represented in base No. 776 to 2632. Cytosine in the nucleotide sequence represented by CpG which is present in the nucleotide sequence of SEQ ID NO: 15 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 470, 472, 490, 497, 504, 506, 509, 514, 522, 540, 543, 552, 566, 582, 597, 610, 612 and so on in the nucleotide sequence of SEQ ID NO: 15 can be recited.

To be more specific, when the useful protein gene is a Solute carrier family 6 neurotransmitter transporter noradrenalin member 2 gene, as a nucleotide sequence that includes at least one nucleotide sequence represented by CpG present in a nucleotide sequence of its promoter region, untranslated region or translated region (coding region), a nucleotide sequence of a genomic DNA containing exon 1 of a Solute carrier family 6 neurotransmitter transporter noradrenalin member 2 gene derived from human, and a promoter region located 5′ upstream of the same can be recited, and more concretely, the nucleotide sequence of SEQ ID NO: 16 (corresponding to a complementary sequence to a nucleotide sequence represented by base No, 78801 to 81000 in the nucleotide sequence described in Genbank Accession No. AC026802) can be recited. In the nucleotide sequence of SEQ ID NO: 16, a nucleotide sequence of exon 1 of a Solute carrier family 6 neurotransmitter transporter noradrenalin member 2 gene derived from human is represented in base No. 1479 to 1804. Cytosine in the nucleotide sequence represented by CpG which is present in the nucleotide sequence of SEQ ID NO: 16 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 1002, 1010, 1019, 1021, 1051, 1056, 1061, 1063, 1080, 1099, 1110, 1139, 1141, 1164, 1169, 1184 and so on in the nucleotide sequence of SEQ ID NO: 16 can be recited.

The term “methylated DNA antibody” in the present measuring method means an antibody that binds to a methylated base in DNA as its antigen. It may be an antibody having a property of recognizing and binding to cytosine methylated at position 5 in single-stranded DNA, and more concretely, a methyl cytosine antibody can be recited. Also a commercially available methylated DNA antibody may be applicable as far as it specifically recognizes and specifically binds to DNA in a methylated state according to the present invention.

A methylated DNA antibody can be prepared by a conventional immunological technique from a methylated base, methylated DNA or the like as an antigen. Concretely, it can be obtained by selecting from antibodies prepared against an antigen such as 5-methylcytidine, 5-methylcytosine or DNA containing 5-methylcytosine according to specific binding to methyl cytosine in DNA as an index.

As an antibody obtainable by immunizing an animal against an antigen, an antibody of an IgG fraction (polyclonal antibody), and an antibody produced by a single clone (monoclonal antibody) are recited. In the present invention, since an antibody capable of specifically recognizing methylated DNA or methyl cytosine is desired, it is preferable to use a monoclonal antibody.

As a method of preparing a monoclonal antibody, a procedure based on a cell fusion method can be recited. For example, in the cell fusion method, a hybridoma is prepared by allowing cell fusion between a pancreatic cell (B cell) derived from an immunized mouse and a myeloma cell, and an antibody produced by the hybridoma is selected, and thus a methyl cytosine antibody (monoclonal antibody) is prepared. When a monoclonal antibody is prepared by a cell fusion method, it is not necessary to purify an antigen, and for example, a mixture of 5-methyl cytidine, 5-methyl cytosine or DNA or the like containing 5-methyl cytosine may be administered as an antigen to an animal used for immunization. As an administration method, 5-methyl cytidine, 5-methyl cytosine or DNA or the like containing 5-methyl cytosine is directly administered to a mouse for production of an antibody. When an antibody is difficult to be produced, an antigen bound to a support may be used for immunization. Also, by thoroughly mixing an adjuvant solution (prepared, for example, by mixing liquid paraffin and Aracel A, and mixing killed tubercle bacilli as an adjuvant) and an antigen, and immunizing via liposome incorporating the same, immunity of an antigen can be improved. Also a method involving adding equivalent amounts of a solution containing an antigen and an adjuvant solution, fully emulsifying them, and subcutaneously or intraperitoneally injecting the resultant mixture to a mouse, and a method of adding killed Bordetella pertussis as an adjuvant after mixing well with alum water are known. A mouse may be boosted intraperitoneally or intravenously after an appropriate term from initial immunization. When the amount of an antigen is small, a solution in which the antigen is suspended may be directly injected into a mouse spleen to effect immunization.

After exenterating a spleen and peeling an adipose tissue off after several days from the final immunization, a spleen cell suspension is prepared. The spleen cell is fused, for example, with an HGPRT-deficient myeloma cell to prepare a hybridoma. As a cell fusion agent, any means capable of efficiently fusing a spleen cell (B cell) and a myeloma cell is applicable, and for example, a method of using a hemagglutinating virus of Japan (HVJ), polyethyleneglycol (PEG) and the like are recited. Cell fusion may be conducted by a method using a high voltage pulse.

After the cell fusion operation, cells are cultured in an HAT medium, a clone of a hybridoma in which a spleen cell and a myeloma cell are fused is selected, and the cell is allowed to grow until screening becomes possible. In a method of detecting an antibody for selecting a hybridoma that produces an intended antibody, or a method of measuring a titer of an antibody, an antigen-antibody reaction system may be used. Concretely, as a method of measuring an antibody against a soluble antigen, a radioisotope immune assay (RIA), an enzyme-linked immunosorbent assay (ELISA) and the like can be recited.

Single-stranded DNA is able to bind with an anti methylation antibody as far as at least one position of a CpG existing therein is methylated. The term “methylated single-stranded DNA” in the present measuring method means single-stranded DNA in which at least one position of a CpG existing in single-stranded DNA is methylated, rather than meaning exclusively single-stranded DNA in which every CpG existing in single-stranded DNA is methylated.

The expression “an amount of amplified DNA obtained by (amplifying methylated DNA in a target DNA region to a detectable level”” means an amount itself after amplification of methylated DNA in a target region comprised by genomic DNA contained in a biological specimen, namely, an amount determined in Third step of the present measuring method. For example, when the biological specimen is 1 mL of serum, it means an amount of DNA amplified based on the methylated DNA contained in 1 mL of serum.

The “methylation-sensitive restriction enzyme” in the present invention, for example, a restriction enzyme or the like that does not digest a recognition sequence containing methylated cytosine, but digests only a recognition sequence containing unmethylated cytosine. In other words, in the case of DNA wherein cytosine contained in a recognition sequence inherently recognizable by the methylation sensitive restriction enzyme is methylated, the DNA will not be cleaved even when the methylation sensitive restriction enzyme is caused to act on the DNA. On the other hand, in the case of DNA wherein cytosine contained in a recognition sequence inherently recognizable by the methylation sensitive restriction enzyme is not methylated, the DNA will be cleaved when the methylation sensitive restriction enzyme is caused to act on the DNA. Concrete examples of such methylation sensitive restriction enzymes include HpaII, BstUI, NarI, SacII, and HhaI. The aforementioned methylation sensitive restriction enzyme will not cleave double-stranded DNA containing a CpG pair in a hemimethyl, state (namely, double-stranded DNA wherein cytosine in one strand is methylated and cytosine in the other strand is not methylated in the above CpG pair) and this is already revealed by Gruenbaum at al. (Nucleic Acid Research, 9, 2509-2515). For example, some of restriction enzymes capable of digesting only a recognition sequence containing unmethylated cytosine without digesting a recognition sequence containing methylated cytosine in single-stranded DNA, namely methylation sensitive restriction enzymes digest single-stranded DNA. As such enzymes, for example, HhaI can be recited.

The term “masking oligonucleotide” used herein means an oligonucleotide comprising a nucleotide sequence recognized by a methylation sensitive restriction enzyme as its part, and is an oligonucleotide that forms a double strand by complementarily binding at least one position (even every position is possible) of recognition sequences of a methylation sensitive restriction enzyme existing at several positions in a target DNA region in single-stranded DNA (namely, the position is made into a double-stranded state), thereby enabling a methylation sensitive restriction enzyme that uses only double-stranded DNA as a substrate to digest the position, and improving digestion efficiency at the position for a methylation sensitive restriction enzyme capable of digesting single-stranded DNA (a methylation sensitive restriction enzyme capable of digesting single-stranded DNA also digests double-stranded DNA, and digestion efficiency thereof is higher with respect to double-stranded DNA than with respect to single-stranded DNA). The present oligonucleotide should be an oligonucleotide that is unusable in a reaction of extending an extension primer using a later-described forward primer or reverse primer as an extension primer and the masking oligonucleotide as a template. As a nucleotide length, a length of 8 to 200 bases is preferred.

The masking oligonucleotide to be mixed with a DNA sample derived from genomic DNA may be one kind or plural kinds. When plural kinds are used, many of recognition sites of a methylation sensitive restriction enzyme in single-stranded DNA containing DNA of a target region come into a double-strand state, and “DNA remaining undigested” as will be described later by a methylation sensitive restriction enzyme can be minimized. For example, it is particularly useful to use a masking oligonucleotide designed for the position intended not to be digested when it is methylated and intended to be digested when it is not methylated among recognition sequences of a methylation sensitive restriction enzyme existing at several positions in a target DNA region (for example, the position that is methylated at 100% in a diseased patient specimen, but is not methylated at 100% in a healthy specimen).

The expression “single-stranded DNA comprising at least one unmethylated CpG in a recognition site of a methylation sensitive restriction enzyme protected by the masking oligonucleotide” used herein means single-stranded DNA in which cytosine in at least one CpG present in a recognition site of the restriction enzyme is unmethylated cytosine.

In First step of the present measuring method, from a DNA sample derived from genomic DNA contained in a biological specimen, methylated single-stranded DNA is selected by binding with an immobilized methylated DNA antibody. Concretely, First step includes First (A) step of separating methylated single-stranded DNA from a DNA sample derived from genomic DNA contained in a biological specimen and First (B) step of selecting the single-stranded DNA by binding between methylated single-stranded DNA and an immobilized methylated. DNA antibody.

In First (A) step of the present measuring method, in “separating methylated single-stranded DNA from a DNA sample derived from genomic DNA contained in a biological specimen”, a commonly used operation for making double-stranded DNA into single-stranded DNA may be conducted. Concretely, a DNA sample derived from genomic DNA contained in a biological specimen may be dissolved in an appropriate amount of ultrapure water, heated at 95° C. for 10 minutes, and rapidly cooled on ice.

The “immobilized methylated DNA antibody” in First (B) step of the present measuring method is used for selecting methylated single-stranded DNA from a DNA sample derived from genomic DNA contained in a biological specimen.

The present immobilized methylated DNA antibody may be one immobilizable to a support, and the expression “one immobilizable to a support” means that a methylated DNA antibody can be immobilized to a support directly or indirectly.

For achieving such immobilization, a methylated DNA antibody may be immobilized to a support according to a commonly used genetic engineering operation method or a commercially available kit, apparatus or the like (binding to a solid phase). Concretely, a method of immobilizing a biotinylated methylated DNA antibody obtained by biotinylating a methylated DNA antibody to a support coated with streptavidin (for example, a PCR tube coated with streptavidin, magnetic beads coated with streptavidin and so on) can be recited.

Also there is a method of letting a molecule having an active functional group such as an amino group, a thiol group, or an aldehyde group covalently bind to a methylated DNA antibody, and letting the resultant bound body covalently bind to a support made of glass, a polysaccharide derivative, silica gel, the synthetic resin or thermostable plastic whose surface is activated with a silane coupling agent or the like. Covalent bonding may be achieved, for example, using a spacer formed by serially connecting five triglycerides, a cross linker or the like.

A methylated DNA antibody may be directly immobilized to a support, or an antibody against a methylated DNA antibody (secondary antibody) may be immobilized to a support, and a methylated antibody may be bound to the secondary antibody to achieve immobilization to a support.

It suffices that the present immobilized methylated DNA antibody is immobilized to a support when single-stranded DNA (plus strand) containing the target DNA region is selected, and (1) immobilization may be achieved by binding between the present immobilized methylated DNA antibody and a support before binding between the single-stranded DNA (plus strand) and the present immobilized methylated DNA antibody, or (2) immobilization may be achieved by binding between the present immobilized methylated DNA antibody and a support after binding between the single-stranded DNA (plus strand) and the present immobilized methylated DNA antibody.

In First (B) step of the present measuring method, when “the single-stranded DNA is selected by binding between methylated single-stranded DNA and an immobilized methylated DNA antibody,” it may be concretely executed in the following manner, for example, using a “biotin-labeled biotinylated methylated cytosine antibody” as an immobilized methylated DNA antibody.

(a) An avidin-coated PCR tube is added with an appropriate amount (for example, 0.1 μg/50 μL) of a biotinylated methylated cytosine antibody, left still at room temperature for about an hour, to promote immobilization between the biotinylated methylated cytosine antibody and streptavidin. Then the remaining solution is removed and washing is performed. A washing buffer [for example, a 0.05% Tween 20-containing phosphate buffer (1 mM KH₂PO₄, 3 mM Na₂HPO.7H₂O, 154 mM NaCl, pH 7.4)] is added in a proportion of 100 μL/tube, and the solution is removed. This washing operation is repeated several times, to leave the biotinylated methylated cytosine antibody immobilized to a support inside the PCR tube.
(b) Double-stranded DNA derived from genomic DNA contained in a biological specimen is mixed with a buffer (for example, 33 mM Tris-Acetate pH 7.9, 66 mM KOAc, 10 mM MgOAc₂, 0.5 mM Dithiothreitol) and heated at 95° C. for several minutes. Then the reaction is rapidly cooled to about 0 to 4° C., and kept for several minutes at this temperature to cause formation of single-stranded DNA. Then the reaction is returned to room temperature.
(c) The formed single-stranded DNA is added to an avidin-coated PCR tube to which a biotinylated methylated cytosine antibody is immobilized, and then left still at room temperature for about an hour, to promote binding between the biotinylated methylated cytosine antibody and methylated single-stranded DNA among the single-stranded DNA (formation of a bound body) (in this stage, at least single-stranded DNA containing an unmethylated DNA region does not form a bound body). Thereafter, the remaining solution is removed and washing is performed. A washing buffer [for example, a 0.05% Tween 20-containing phosphate buffer (1 mM KH₂PO₄, 3 mM Na₂HPO.7H₂O, 154 mM NaCl, pH 7.4)] is added in a proportion of 100 μL/tube, and the solution is removed. This washing operation is repeated several times, to leave the bound body inside the PCR tube (selection of a bound body).

The buffer used in (b) is not limited to the above buffer and may be any buffer that is suited for separating double-stranded DNA derived from genomic DNA from a biological sample into single-stranded DNA.

The washing operation in (a) and (c) is important for removing an unimmobilized methylated DNA antibody suspended in the solution, unmethylated single-stranded DNA that does not bind with a methylated DNA antibody and hence is suspended in the solution, and DNA suspended in a solution digested by a restriction enzyme as will be described later, from the reaction solution. The washing buffer is not limited to the foregoing washing buffer, and any buffer suited for removing the free methylated DNA antibody, single-stranded DNA and so on suspended in the solution and the like is applicable, and a DELFIA buffer (available from Perkin Elmer, Tris-HCl pH 7.8 with Tween 80), a TE buffer and the like may be used.

In Second step of the present measuring method, single-stranded DNA selected in First step is digested by a methylation sensitive restriction enzyme recognizing single-stranded DNA as a substrate, however, it is not digested by a majority of methylation sensitive restriction enzymes that recognize only double-stranded DNA as a substrate. Therefore, by mixing single-stranded DNA selected in First step with an oligonucleotide comprising the nucleotide sequence that recognizes a methylation sensitive restriction enzyme as a masking oligonucleotide, the methylation sensitive restriction enzyme that recognizes only double-stranded DNA as a substrate is enabled to digest a recognition sequence of a methylation sensitive restriction enzyme having an unmethylated CpG, and digestion efficiency of the site by a methylation sensitive restriction enzyme capable of digesting single-stranded DNA is improved.

The masking oligonucleotide may be mixed before separating genomic DNA into single strand, or may be mixed in the state of single-stranded DNA selected in First step as a previous step of Second step, or alternatively, it may be mixed after binding with an immobilized methylated DNA antibody, as far as it binds with a nucleotide sequence recognizable by a methylation sensitive restriction enzyme and forms a double strand therewith before a treatment with the methylation sensitive restriction enzyme.

In Second step of the present measuring method, single-stranded DNA selected in First step is mixed with a masking oligonucleotide comprising the nucleotide sequence complementary to a nucleotide sequence of a recognition site of a methylation sensitive restriction enzyme, and the selected single-stranded DNA is digested with at least one kind of methylation sensitive restriction enzyme, and then generated free digests (single-stranded DNA containing at least one unmethylated CpG in a recognition site of a methylation sensitive restriction enzyme protected by the masking oligonucleotide) are removed.

As a method of examining occurrence of digestion by the methylation sensitive restriction enzyme in the condition that the masking oligonucleotide is bound thereto, concretely, a method of conducting PCR using the single-stranded DNA as a template, and a pair of primers capable of amplifying DNA containing cytosine in a recognition sequence to be analyzed, and examining amplification of DNA (amplification product) can be recited as an example. When cytosine to be analyzed is methylated, an amplification product is obtained. On the other hand, when cytosine to be analyzed is not methylated, an amplification product is not obtained. In this manner, by comparing the amplified DNA amount, it is possible to determine the methylated rate of cytosine to be analyzed.

By the way, in Second step, since the masking oligonucleotide is added to the selected single-stranded DNA, the target DNA region to which the masking oligonucleotide is bound is in a double-stranded state. In this double strand, the minus strand side is a masking oligonucleotide, and cytosine contained in the masking oligonucleotide is not methylated. On the other hand, the plus strand side is genomic DNA contained in a biological specimen, and whether the state of the double-stranded DNA is unmethylated is determined depending on whether cytosine contained in the genomic DNA is originally methylated or not. In other words, when genomic DNA contained in a biological specimen is methylated, the double-stranded DNA part to which the masking oligonucleotide is bound is in a hemimethyl state (the state not being unmethylated, minus strand: unmethylated state, plus strand: methylated state), and when genomic DNA contained in a biological specimen is not methylated, the double-stranded DNA part to which the masking oligonucleotide is bound is in a non-methyl state (minus strand: unmethylated, plus strand: unmethylated). Therefore, by utilizing the characteristic that the aforementioned methylation sensitive restriction enzyme does not cleave double-stranded DNA in a hemimethyl state, it is possible to distinguish whether cytosine in at least one CpG pair present in the recognition site of a methylation sensitive restriction enzyme in genomic DNA in the biological specimen is methylated or not. That is, by digesting with the methylation sensitive restriction enzyme, if cytosine in at least one CpG pair present in the double-stranded DNA part to which the masking oligonucleotide is bound in genomic DNA contained in the biological specimen is not methylated, the double-stranded DNA part to which the masking oligonucleotide is bound is in a non-methyl state, and cleaved by the methylation sensitive restriction enzyme. If cytosine in every CpG pair present in the double-stranded DNA part to which the masking oligonucleotide is bound in genomic DNA contained in the biological specimen is methylated, the double-stranded DNA part to which the masking oligonucleotide is bound is in a hemimethyl state, and will not be cleaved by the methylation sensitive restriction enzyme. Therefore, by executing PCR using a pair of primers capable of amplifying the target DNA region as will be described later after executing a digestion treatment after binding a masking oligonucleotide to a recognition sequence of a methylation sensitive restriction enzyme in a target DNA region, an amplification product by PCR will not be obtained when cytosine in at least one CpG pair present in the double-stranded DNA part to which the masking oligonucleotide is bound in genomic DNA contained in the biological specimen is not methylated, whereas an amplification product by PCR will be obtained when cytosine in every CpG pair present in the double-stranded DNA part to which the masking oligonucleotide is bound in genomic DNA contained in the biological specimen is methylated.

As a concrete example of a methylation sensitive restriction enzyme, HpaII or HhaI is recited, and by using such an enzyme by adding a masking oligonucleotide, it is possible to determine whether single-stranded DNA is methylated or not. That is, when cytosine of a CpG contained in a recognition site of HpaII or HhaI in the single-stranded part of DNA obtained through the above operations is methylated, HpaII or HhaI is unable to digest the DNA, and when it is not methylated, HpaII or HhaI digests the DNA. Therefore, when a PCR reaction using a pair of primers capable of amplifying the target DNA region after execution of the above reaction, an amplification product will not be obtained if DNA in the specimen is unmethylated, and an amplification product will be obtained if DNA in the specimen is in a methylated state.

In Second step of the present measuring method, a digestion treatment is conducted by adding a masking oligonucleotide, and at least one kind of methylation sensitive restriction enzyme capable of digesting a recognition sequence of a methylation sensitive restriction enzyme protected by the masking oligonucleotide, and incubating the mixture at 37° C. for an hour to overnight. Concretely, for example, it may be executed in the following manner. Single-stranded DNA selected in First step is added with 3 μL of an optimum buffer (330 mM Tris-Acetate pH 7.9, 660 mM KOAc, 100 mM MgOAc₂, 5 mM Dithiothreitol), 1.5 μL of a methylation sensitive restriction enzyme HhaI (10 U/μL) capable of digesting single-stranded DNA, each about 10 pmol of masking oligonucleotides for recognition sequences of the methylation sensitive restriction enzymes, and an appropriate amount of other substances such as BSA as necessary, and then the resultant mixture is added with sterile ultrapure water to a liquid volume of 30 μL, followed by incubation at 37° C. for an hour to overnight. As a result, when a recognition site (position) of the methylation sensitive restriction enzyme protected by the masking oligonucleotide in the target DNA region is not methylated, the position will be digested.

After digesting the double-stranded DNA formed in first step with at least one kind of methylation sensitive restriction enzyme, the generated free digest (single-stranded DNA containing at least one unmethylated CpG in the recognition site of the methylation sensitive restriction enzyme protected by the masking oligonucleotide) is removed and washed (DNA purification).

More concretely, for example, when a PCR tube coated with streptavidin is used, after removing the solution by pipetting or decantation first, a TE buffer of an amount approximately equal to the volume of the biological specimen is added, and thereafter, the TE buffer may be removed by pipetting or decantation. When magnetic beads coated with streptavidin are used, after immobilizing the beads by a magnet, the solution is removed by pipetting or decantation first, and a TE buffer of an amount approximately equal to the volume of the biological specimen is added, and thereafter, the TE buffer may be removed by pipetting or decantation.

Then, by executing such an operation several times, the digest (single-stranded DNA containing at least one unmethylated CpG in the recognition site of the restriction enzyme) is removed and washed (DNA purification).

As a fear in a digestion treatment with a methylation sensitive restriction enzyme in Second step of the present measuring method, the possibility that the recognition sequence containing unmethylated cytosine may not be completely digested (so called “DNA remaining undigested”) is recited. When such a possibility is problematic, the target DNA region has at least one recognition site of a methylation sensitive restriction enzyme, and as many as possible recognition sites would be preferred because it is possible to suppress the “DNA remaining undigested” as much as possible by presence of abundant recognition sites of a methylation sensitive restriction enzyme capable of digesting single-stranded DNA. Here, a larger number of recognition sites of a methylation sensitive restriction enzyme protected by the masking oligonucleotide would be preferred.

Therefore, when a treatment using plural kinds of methylation sensitive restriction enzymes is executed in Second step of the present measuring method, concretely, the treatment may be executed in the following manner. Single-stranded DNA selected in First step is added with 3 μL of an optimum buffer (330 mM Tris-Acetate pH 7.9, 660 mM KOAc, 100 mM MgOAc₂, 5 mM Dithiothreitol), each 1.5 μL of methylation sensitive restriction enzymes HpaII and HhaI (10 U/μL) or the like, each about 10 pmol of masking oligonucleotides for recognition sequences of the several kinds of methylation sensitive restriction enzymes, and an appropriate amount of other substances such as BSA as necessary, and the resultant mixture is added with sterile ultrapure water to a liquid volume of 30 μL, and incubated at 37° C. for an hour to overnight. When the recognition site (position) of the methylation sensitive restriction enzyme protected by the masking oligonucleotide in the target DNA region is not methylated, the position will be digested. Thereafter, according to an operation similar to that as described above, the remaining solution is removed and washing is performed by pipetting or decantation (DNA purification). More concretely, for example, when a PCR tube coated with streptavidin is used, after removing the solution by pipetting or decantation first, a washing buffer of an amount approximately equal to the volume of the biological specimen is added, and thereafter, the buffer may be removed by pipetting or decantation. When magnetic beads coated with streptavidin are used, the solution is removed by pipetting or decantation first after immobilizing the beads by a magnet, and a washing buffer of an amount approximately equal to the volume of the biological specimen is added, and thereafter, the washing buffer may be removed by pipetting or decantation.

In the present measuring method, one preferable exemplary embodiment is that “a DNA sample derived from genomic DNA contained in a biological specimen” is a DNA sample preliminarily digested with a restriction enzyme whose recognition cleavage site excludes the target DNA region comprised by the genomic DNA. Here, when genomic DNA contained in a biological specimen (template DNA) is selected with the use of the present immobilized oligonucleotide, shorter template DNA is more likely to be selected, and when the target region is amplified by PCR, shorter template DNA is more preferred. Therefore, a digestion treatment may be executed using a restriction enzyme whose recognition cleavage site excludes the target DNA region directly on the DNA sample derived from genomic DNA contained in a biological specimen. As a method of digesting with a restriction enzyme whose recognition cleavage site excludes the target DNA region, a commonly used restriction enzyme treatment method may be used.

One exemplary preferable embodiment is that “a DNA sample derived from genomic DNA contained in a biological specimen” is a DNA sample digested with at least one kind of a methylation sensitive restriction enzyme.

These preferred embodiments enable accurate determination of the methylation amount by preliminarily digesting the biological specimen itself with a restriction enzyme as described above. Such a method is useful for avoiding the “DNA remaining undigested” as described above.

As a method of digesting a sample derived from genomic DNA contained in a biological specimen with a methylation sensitive restriction enzyme, when the biological specimen is genomic DNA itself, the method similar to that described above is preferred. When the biological specimen is a tissue lysate, a cell lysate or the like, a digestion treatment may be executed using a large excess of a methylation sensitive restriction enzyme, for example, a methylation sensitive restriction enzyme in an amount of 500 times (10 U) or more with respect to 25 ng of the DNA amount, according to a method similar to that described above.

It is believed that genomic DNA basically exists in the form of double-stranded DNA. Therefore, in the present operation, a digestion treatment may be executed by a general method without adding a masking oligonucleotide.

For example, free DNA contained in blood or the like may be single-stranded DNA, and DNA contained in a certain virus particle is single strand DNA. In such a case, when a digestion treatment is executed using a restriction enzyme that digests only double-stranded DNA, a masking oligonucleotide containing a recognition sequence of the restriction enzyme may be used.

In Third step of the present measuring method, Step (Third (pre A) step) of separating single-stranded DNA (single-stranded DNA not containing an unmethylated CpG in a recognition site of a methylation sensitive restriction enzyme protected by the masking oligonucleotide) which is an undigested substance obtained in Second step from the Immobilized methylated DNA antibody into free single-stranded DNA;

Step (Third (pre B) step) of extensionally forming double-stranded DNA from free single-stranded DNA (plus strand) by extending DNA (plus strand) derived from a genome that is made into a free single-stranded state in Third (pre A) step by one extension of an extension primer using the extension primer (forward primer) comprising a nucleotide sequence (minus strand) which is complementary to a partial nucleotide sequence (plus strand) of the nucleotide sequence comprised by the single-stranded DNA (plus strand), the partial nucleotide sequence (plus strand) located on further 3′-end side than 3′-end of the nucleotide sequence (plus strand) of the target DNA region, as the extension primer; and

Step (Third (pre C) step) of temporarily separating the double-stranded DNA extensionally formed in Third (pre B) step into single-stranded DNA (plus strand) containing the target DNA region and single-stranded DNA (minus strand) containing the target DNA region are included as pre steps of the following regular steps, and

(a) Third step-A of extensionally forming double-stranded DNA from single-stranded DNA containing the target DNA region by one extension of an extension primer by using the generated single-stranded DNA (plus strand) containing the target DNA region as a template, and the forward primer as an extension primer, and

(b) Third step-B of extensionally forming double-stranded DNA from single-stranded DNA containing the target DNA region by one extension of an extension primer by using the generated single-stranded DNA (minus strand) containing the target DNA region as a template, and an extension primer (reverse primer) comprising a nucleotide sequence (plus strand) which is complementary to a partial nucleotide sequence (minus strand) of the nucleotide sequence comprised by the single-stranded DNA (minus strand) containing the target DNA region, the partial nucleotide sequence (minus strand) located on further 3′-end side than 3′-end of the nucleotide sequence (minus strand) complementary to the nucleotide sequence (plus strand) of the target DNA region as the extension primer, are included as regular steps, and by repeating the regular steps after temporarily separating the extensionally formed double-stranded DNA obtained in each of the regular steps into a single-stranded state, the methylated DNA in the target DNA region is amplified to a detectable level and a content of the amplified DNA is quantified.

In Third step of the present measuring method, first, as Third (pre A) step among the respective pre steps of the following regular steps, single-stranded DNA (single-stranded DNA not containing an unmethylated CpG in a recognition site of a methylation sensitive restriction enzyme protected by the masking oligonucleotide) which is an undigested substance obtained in Second step is temporarily separated from the immobilized methylated DNA antibody into DNA in a single-stranded state. Concretely, for example, by adding an annealing buffer to single-stranded DNA (single-stranded DNA not containing an unmethylated CpG in a recognition site of a methylation sensitive restriction enzyme protected by the masking oligonucleotide) which is an undigested substance obtained in Second step, a mixture is obtained. Then the resultant mixture is heated at 95° C. for several minutes, to obtain DNA in a single-stranded state (plus strand). Thereafter, in Third (pre B) step, concretely, for example, DNA in a single-stranded state (plus strand) obtained in Third (pre A) step and a forward primer are mixed in a solution that is prepared by adding 17.85 μL of sterile ultrapure water, 3 μL of an optimum buffer (for example, 100 mM Tris-HCl pH 8.3, 500 mM KCl, 15 mM MgCl₂), 3 μL of 2 mM dNTP, and 6 μL of 5 N betaine, and adding the resultant mixture with 0.15 μL, of AmpliTaq (a kind of DNA polymerase: 5 U/μL) to a liquid volume of 30 μL, and incubated at 37° C. for about two hours, to extensionally form double-stranded DNA from the single-stranded DNA (plus strand) containing a target DNA region. In Third (pre C) step, concretely, for example, the double-stranded DNA extensionally formed in Third (pre B) step is added with an annealing buffer to obtain a mixture, and the DNA is temporarily separated into single-stranded DNA containing the target DNA region by heating the mixture at 95° C. for several minutes.

Thereafter, the following regular steps are conducted.

(i) The reaction is rapidly cooled to a temperature lower than Tm of the forward primer by about 0 to 20° C., and kept at this temperature for several minutes for annealing the forward primer to the generated single-stranded DNA (plus strand) containing the target DNA region.
(ii) Thereafter, the reaction is returned to room temperature.
(iii) Double-stranded DNA is extensionally formed from single-stranded DNA containing the target DNA region by one extension of an extension primer by using the DNA in a single-stranded state annealed in the above (i) as a template, and the forward primer as an extension primer (namely, Third step-A). Concretely, it may be executed, for example, according to the later-described explanation, or the operation method in an extension reaction in Third (pre B) step of the present measuring method as described above.
(iv) Single-stranded DNA is made into extensionally formed double-stranded DNA by one extension of an extension primer by using the generated single-stranded DNA (minus strand) containing the target DNA region as a template, and an extension primer (reverse primer) comprising a nucleotide sequence (plus strand) which is complementary to a partial nucleotide sequence (minus strand) of the nucleotide sequence comprised by the single-stranded DNA (minus strand) containing the target DNA region, the partial nucleotide sequence (minus strand) located on further 3′-end side than 3′-end of the nucleotide sequence (minus strand) complementary to the nucleotide sequence (plus strand) of the target DNA region as the extension primer (namely, Third step-B). Concretely, it may be executed, for example, according to the operation method in an extension reaction in Third (pre B) step similarly to Third step-A of the above (iii),
(v) By repeating the regular steps of Third step after temporarily separating the extensionally formed double-stranded DNA obtained in each of the regular steps into a single-stranded state (for example, Third step-A and Third step-B), the methylated DNA in the target DNA region is amplified to a detectable level and a content of the amplified DNA is quantified.

In Third step, concretely the reaction starting from Third (pre A) step and up to regular steps may be executed as a single PCR reaction. Also, from Third (pre A) step to Third (pre C) step, each reaction may be independently executed, and only regular steps may be executed as a PCR reaction.

As a method of amplifying a target DNA region (namely, a target region) after digestion with a methylation sensitive restriction enzyme in the condition of being bound to a masking oligonucleotide, for example, PCR may be used. Using a primer preliminarily labeled with fluorescence or the like and utilizing the label as an index in amplifying the target region make it possible to evaluate presence or absence of an amplification product without executing a burdensome operation such as electrophoresis. As a PCR reaction solution, for example, a reaction solution obtained by mixing DNA obtained in Second step of the present measuring method with 0.15 μL of a 50 μM primer solution, 2.5 μL of 2 mM dNTP, 2.5 μL of a 10× buffer (100 mM Tris-HCl pH 8.3, 500 mM KCl, 20 mM MgCl₂, 0.01% Gelatin), and 0.2 μL of AmpliTaq Gold (one kind of thermostable DNA polymerase: 5 U/μL), and adding sterilized ultrapure water to make the liquid volume 25 μL can be recited.

Since a target DNA region (namely, a target region) often has a GC rich nucleotide sequence, the reaction may be occasionally executed with addition of an appropriate amount of betaine, DMSO or the like. In one exemplary reaction condition, the reaction solution as described above is kept at 95° C. for 10 minutes, and then a cycle including incubation of 30 seconds at 95° C., 30 seconds at 55 to 65° C., and 30 seconds at 72° C. is repeated 30 to 40 times. After conducting such PCR, the obtained amplification product is detected. For example, when a preliminarily labeled primer is used, an amplification amount by a PCR reaction can be evaluated by measuring an amount of a fluorescent label after executing washing and purification operations similar to those as described above. When PCR is conducted using a normal primer that is not labeled, a probe or the like that is labeled with a gold colloid particle, fluorescence or the like is caused to anneal, and detection may be achieved by measuring an amount of the probe bound to the target region. Also, for determining an amount of an amplification product more accurately, for example, a real time PCR method may be used. Real time PCR is a method in which PCR is monitored in real time, and the obtained monitor result is analyzed kinetically, and is known as a high-accuracy quantitative PCR method capable of detecting a very small difference of as small as twice in a gene amount. As such a real time PCR method, for example, a method using a probe such as a template-dependent nucleic acid polymerase probe, a method using an intercalator such as SYBR-Green and the like can be recited. As an apparatus and a kit for the real time PCR method, those commercially available may be used. As described above, detection may be executed by any method conventionally well-known without any particular limitation. These methods make it possible to conduct the operations up to detection without requiring change of the reaction container.

As another embodiment of Second step, executing Third step without executing a digestion treatment with a methylation sensitive restriction enzyme can be recited. When there is no nucleotide sequence that is cleaved by a methylation sensitive restriction enzyme in the target DNA region, Third step may be executed without executing Second step.

In First (A) step, as a preferred embodiment in separating methylated single-stranded DNA, addition of a counter oligonucleotide and the like can be recited. A counter oligonucleotide means a short oligonucleotide comprising the same nucleotide sequence as that of the target DNA region. It may be designed to have a length of usually 10 to 100 bases, and more preferably 20 to 50 bases. Here, a counter oligonucleotide is not designed on the nucleotide sequence where a forward primer or a reverse primer complementarily binds with the target DNA region. A counter oligonucleotide is added in excess relative to genomic DNA, and is added so as to prevent a complementary strand of a target DNA region (minus strand) and a single strand of a target DNA region (plus strand) from re-binding by complementation when binding with an immobilized methylated DNA antibody is caused after making a target DNA region into a single strand (plus strand). This is because in measuring a methylation frequency of DNA or an index value having correlation therewith while a methylated DNA antibody is bound to the target DNA region, the target region is more likely to bind with the methylated DNA antibody when it is a single strand. Preferably, a counter oligonucleotide is added in an amount of at least 10 times, usually 100 times or more relative to the target DNA region.

“Adding a counter oligonucleotide in separating methylated single-stranded DNA” may be concretely achieved by mixing a DNA sample derived from genomic DNA contained in a biological specimen with a counter oligonucleotide to form a double strand between the complementary strand of the target DNA region and the counter oligonucleotide so as to select methylated single-stranded DNA from a DNA sample derived from genomic DNA contained in a biological specimen. For example, the DNA sample and the counter oligonucleotide are added to 5 μL of a buffer (330 mM Tris-Acetate pH 7.9, 660 mM KOAc, 100 mM MgOAc₂, 5 mM Dithiothreitol), 5 μL of a 100 mM MgCl₂ solution, and 5 μL of a 1 mg/mL of BSA solution, and the resultant mixture is added with sterile ultrapure water to a liquid volume of 50 μL, and mixed, heated at 95° C. for 10 minutes, rapidly cooled to 70° C., kept at this temperature for 10 minutes, then cooled to 50° C., kept at this temperature for 10 minutes, and then kept at 37° C. for 10 minutes and returned to room temperature.

The present measuring method may be used in the following situations.

It is known that DNA methylation abnormality occurs in various diseases (for example, cancer), and it is believed that the degree of various diseases can be measured by detecting this DNA methylation abnormality.

For example, when there is a DNA region where methylation occurs at 100% in genomic DNA contained in a diseased biological specimen, and the present measuring method is executed for the DNA region, the amount of methylated DNA will increase. For example, when there is a DNA region where methylation does not occur at 100% in genomic DNA contained in a diseased biological specimen, and the present measuring method is executed for the DNA region, the amount of methylated DNA will be approximately 0. For example, when there is a DNA region which is in hypomethylation in genomic DNA contained in a specimen derived from a healthy subject, and in hypermethylation in genomic DNA contained in a specimen derived from a disease subject, and the present measuring method is executed for the DNA region, the amount of methylated DNA would be approximately 0 for the healthy subject, and a significantly higher value than that of the healthy subject will be exhibited by the disease patient, so that the “degree of disease” can be determined based on this difference in value. The “degree of disease” used herein has the same meaning commonly used in this field of art, and concretely means, for example, malignancy when the biological specimen is a cell, and means, for example, abundance of disease cells in the tissue when the biological specimen is a tissue. Further, when the biological specimen is plasma or serum, it means the probability that the individual has the disease. Therefore, the present measuring method makes it possible to diagnose various diseases by examining methylation abnormality.

Restriction enzymes, primers or probes that can be used in various methods for measuring a methylated DNA amount in a target region in the present measuring method are useful as reagents of a detection kit. The present invention also provides a detection kit containing these restriction enzymes, primers or probes as reagents, and a detection chip in which these primers, probes and so on are immobilized on a support, and a scope of the present measuring method or the present methylation rate measuring method of course embraces use in the form of a detection kit or a detection chip as described above utilizing the substantial principle of the method.

### EXAMPLES

In the following, the present invention will be explained in detail by way of examples, however, the present invention will not be limited to these examples.

### Example 1

A commercially available methylated cytosine antibody (available from Aviva Systems Biology) was labeled with biotin using a commercially available biotinylating kit (Biotin Labeling Kit-NH₂, available from DOJINDO Laboratories) according to the method described in the catalogue. The obtained biotin-labeled methylated cytosine antibody was refrigerated as a solution [about 0.1 μg/50 μL solution of an antibody in a 0.1% BSA-containing phosphate buffer (1 mM KH₂PO₄, 3 mM Na₂HPO.7H₂O, 154 mM NaCl, pH 7.4)].

To each PCR tube coated with streptavidin (a total of 9 tubes), 50 μL of the biotin-labeled methylated cytosine antibody solution was added and immobilized to the PCR tube by leaving it still for about an hour at room temperature. Then, after removing the solution in the PCR tube by pipetting, 100 μL of a washing buffer [0.05% Tween 20-containing phosphate buffer (1 mM KH₂PO₄, 3 mM Na₂HPO.7H₂O, 154 mM NaCl, pH 7.4)] was added into the PCR tube. Then the buffer in the PCR tube was removed by pipetting. This operation was repeated another two times.

A partially methylated oligonucleotide GPR-2079-2176/98 mer-M(7) in which a recognition site of HpaII comprising the nucleotide sequence of SEQ ID NO: 17 is methylated; a partially methylated oligonucleotide GPR7-2079-2176/98 mer-HM(5) in which part of a recognition site of HpaII having the nucleotide sequence of SEQ ID NO: 18 is not methylated; and an unmethylated oligonucleotide GPR-2079-2176/98 mer-UM having the nucleotide sequence of SEQ ID NO: 19 were separately synthesized, and a 0.01 pmol/50 μL solution in a TE buffer (hereinafter, referred to as an oligonucleotide solution) was prepared for each oligonucleotide.

<Partially Methylated Oligonucleotide in which Recognition Sequence of HpaII is Methylated>
N denotes methylated cytosine.

(SEQ ID NO: 17)

GPR7-2079-2176/98mer-M(7):
5′-
gttggccactgcggagtcgngcngggtggcnggccgcacctacagngccg
ngngngcggtgagcctggccgtgtgggggatcgtcacactcgtcgtgc-
3′

<Partially Methylated Oligonucleotide in which Recognition Sequence of HpaII is not Methylated>
N denotes methylated cytosine.

(SEQ ID NO: 18)

GPR7-2079-2176/98mer-HM(5):
5′-
gttggccactgcggagtcgcgccgggtggcnggccgcacctacagngccg
ngngngcggtgagcctggccgtgtgggggatcgtcacactcgtcgtgc-
3′

### <Unmethylated Oligonucleotide>

(SEQ ID NO: 19)

GPR7-2079-2176/98mer-UM:
5′-
gttggccactgcggagtcgcgccgggtggccggccgcacctacagcgccg
cgcgcgcggtgagcctggccgtgtgggggatcgtcacactcgtcgtgc-
3′

Each of the obtained solutions was subjected to the following treatment (Each solution was prepared in triplicate).

The PCR tube coated with streptavidin to which a biotin-labeled methylated cytosine antibody was immobilized was added with 50 μL, of the oligonucleotide solution prepared as described above and left still for an hour at room temperature. Then the solution in the tube was removed by pipetting, and the PCR tube was added with 100 μL of a washing buffer [0.05% Tween 20-containing phosphate buffer (1 mM KH₂PO₄, 3 mM Na₂HPO.7H₂O, 154 mM NaCl, pH 7.4)]. Thereafter, the buffer in the PCR tube was removed by pipetting. This operation was repeated another two times (these correspond to First step of the present measuring method).

The samples obtained in this manner were subjected to an A treatment, a B treatment or a C treatment (each prepared singly).

A treatment group (no treatment group): The sample prepared above was added with 10 μL of a buffer (330 mM Tris-Acetate pH 7.9, 660 mM KOAc, 100 mM MgOAc₂, 5 mM Dithiothreitol), and 10 μL of BSA (Bovine serum albumin 1 mg/mL), and the resultant mixture was added with sterile ultrapure water to a liquid volume of 100 μL.

B treatment group (HpaII treatment group): The sample prepared above was added with 10 μL of a buffer (330 mM Tris-Acetate pH 7.9, 660 mM KOAc, 100 mM MgOAc₂, 5 mM Dithiothreitol), 10 μL of BSA (Bovine serum albumin 1 mg/mL), and 10 U of HpaII, and the resultant mixture was added with sterile ultrapure water to a liquid volume of 100 μL.

C treatment group (addition of masking oligonucleotide+HpaII treatment group): The sample prepared above was added with 10 μL of a buffer (330 mM Tris-Acetate pH 7.9, 660 mM KOAc, 100 mM MgOAc₂, 5 mM Dithiothreitol), 10 μL of BSA (Bovine serum albumin 1 mg/mL), 10 U of HpaII, and 5 pmol of an oligonucleotide MA (masking oligonucleotide) comprising the nucleotide sequence of SEQ ID NO: 20, and the resultant mixture was added with sterile ultrapure water to a liquid volume of 100 μL.

### <Masking Oligonucleotide>

| | | |
|---|---|---|
| MA: | 5′-gccacccggcgcga-3′ | (SEQ ID NO: 20) |

Each reaction mixture was incubated overnight at 37° C. Then, after removing the solution in the PCR tube by pipetting, the PCR tube was added with 100 μL of a washing buffer [0.05% Tween 20-containing phosphate buffer (1 mM KH₂PO₄, 3 mM Na₂HPO.7H₂O, 154 mM NaCl, pH 7.4)]. Thereafter, the buffer in the PCR tube was removed by pipetting. This operation was repeated another two times (these correspond to Second step of the present measuring method).

Next, the PCR tube obtained in this manner was subjected to PCR using solutions of primers (PF2 and PR2) comprising the nucleotide sequences of SEQ ID NO: 21 and SEQ ID NO: 22, and the following reaction condition, to amplify methylated DNA in a target DNA region (GPR7-2079-2176, SEQ ID NO: 23, methylated cytosine is also denoted by C).

### <Primers>

| | | |
|---|---|---|
| PF2: | 5′-gttggccactgcggagtcg-3′ | (SEQ ID NO: 21) |
| PR2: | 5′-gcacgacgagtgtgacgatc-3′ | (SEQ ID NO: 22) |

### <Target DNA Region>

(SEQ ID NO: 23)

GPR7-2079-2176:
5′-
gttggccactgcggagtcgcgccgggtggccggccgcacctacagcgccg
cgcgcgcggtgagcctggccgtgtgggggatcgtcacactcgtcgtgc-
3′

A reaction solution of PCR was prepared by mixing each 5 μL of solutions of primers comprising the nucleotide sequences of SEQ ID NO: 21 and SEQ ID NO: 22 prepared to 3 μM, each 5 μL of 2 mM dNTPs, 5 μL of a buffer (100 mM Tris-HCl pH 8.3, 500 mM KCl, 15 mM MgCl₂, 0.01% Gelatin), 0.25 μL of 5 U/μL thermostable DNA polymerase (AmpliTaq Gold), and 10 μL of a 5 N betaine aqueous solution to DNA which is a template, and adding sterile ultrapure water to a liquid volume of 50 μL. The reaction solution was kept at 95° C. for 10 minutes, and then subjected to PCR conducting 25 cycles of incubation each including 30 seconds at 95° C., 30 seconds at 59° C., and 45 seconds at 72° C.

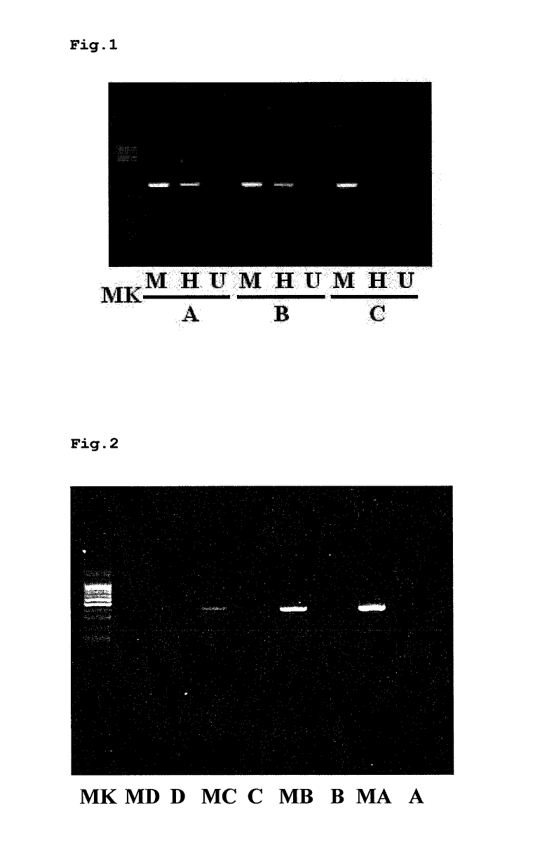

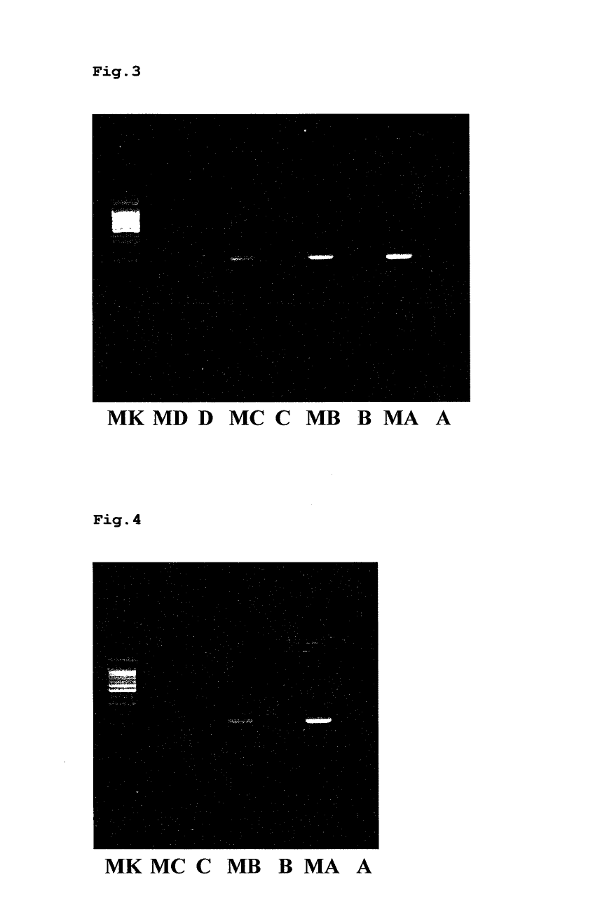

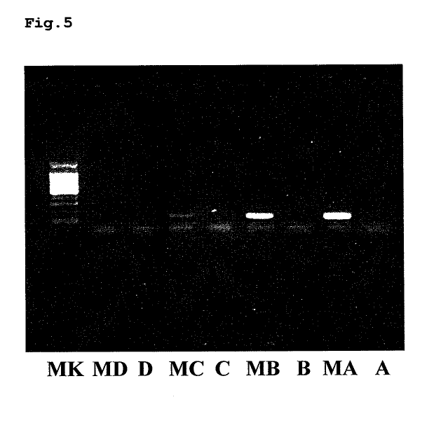

After conducting PCR, the obtained amplification product was subjected to 1.5% agarose gel electrophoresis to check the amplification of DNA. The result is shown in FIG. 1 (these correspond to Third step of the present measuring method).

In the case of A treatment group (no treatment group), in the partially methylated oligonucleotide GPR7-2079-2176/98 mer-M(7) in which the recognition sequence of HpaII is methylated, and the partially methylated oligonucleotide GPR7-2079-2176/98 mer-HM(5) in which part of the recognition sequence of HpaII is not methylated, amplification of DNA was observed, and an amplification product thereof (target DNA region: GPR7-2079-2176) was obtained. In the unmethylated oligonucleotide GPR7-2079-2176/98 mer-UM, amplification of DNA was not observed, and an amplification product thereof was not obtained. Also in the case of B treatment group (HpaII treatment group), the result was similar to that in A treatment group. In the case of C treatment group (addition of masking oligonucleotide+HpaII treatment group), in the partially methylated oligonucleotide GPR7-2079-2176/98 mer-M(7) in which the recognition sequence of HpaII is methylated, amplification of DNA was observed, and an amplification product thereof (target DNA region: GPR7-2079-2176) was obtained. Contrarily, in the cases of the partially methylated oligonucleotide GPR7-2079-2176/98 mer-HM(5) in which part of the recognition sequence of HpaII is not methylated, and in the unmethylated oligonucleotide GPR7-2079-2176/98 mer-UM, amplification of DNA was not observed, and an amplification product thereof was not obtained.

### Example 2

A commercially available methylated cytosine antibody (available from Aviva Systems Biology) was labeled with biotin using a commercially available biotinylating kit (Biotin Labeling Kit-NH₂, available from DOJINDO Laboratories) according to the method described in the catalogue. The obtained biotin-labeled methylated cytosine antibody was refrigerated as a solution [about 0.1 μg/100 μL solution of an antibody in a 0.1% BSA-containing phosphate buffer (1 mM KH₂PO₄, 3 mM Na₂HPO.7H₂O, 154 mM NaCl, pH 7.4)].

To each PCR tube coated with streptavidin (a total of 8 tubes), 50 μL of the synthetically obtained biotin-labeled methyl cytosine antibody solution was added and immobilized to the PCR tube by leaving it still for about an hour at room temperature. Then, after removing the solution by pipetting, 100 μL of a washing buffer [0.05% Tween 20-containing phosphate buffer (1 mM KH₂PO₄, 3 mM Na₂HPO.7H₂O, 154 mM NaCl, pH 7.4)] was added, and then the buffer was removed by pipetting. This operation was repeated another two times (these correspond to preparation of an immobilized methylated DNA antibody used in the present measuring method).

For genomic DNA derived from human blood purchased from Clontech, a DNA fragment (X, SEQ ID NO: 26, a region corresponding to the base numbers 25687390 to 25687775 shown in Genbank Accession No. NT 029419 and so on) to be used as a test sample was amplified by conducting PCR using oligonucleotide primers (PF1 and PR1) designed for PCR of SEQ ID NO: 24 and SEQ ID NO: 25 and the following reaction condition.

### <Oligonucleotide Primers Designed for PCR>

| | | |
|---|---|---|
| PF1: | 5′-CTCAGCACCCAGGCGGCC-3′ | (SEC ID NO: 24) |
| PR1: | 5′-CTGGCCAAACTGGAGATCGC-3′ | (SEQ ID NO: 25) |

### <DNA Fragment>

(SEQ ID NO: 26)

X: 5′-
CTCAGCACCCAGGCGGCCGCGATCATGAGGCGCGAGCGGCGCGCGGGCTG
TTGCAGAGTCTTGAGCGGGTGGCACACCGCGATGTAGCGGTCGGCTGTCA
TGACTACCAGCATGTAGGCCGACGCAAACATGCCGAACACCTGCAGGTGC
TTCACCACGCGGCACAGCCAGTCGGGGCCGCGGAAGCGGTAGGTGATGTC
CCAGCACATTTGCGGCAGCACCTGGAAGAATGCCACGGCCAGGTCGGCCA
GGCTGAGGTGTCGGATGAAGAGGTGCATGCGGGACGTCTTGCGCGGCGTC
CGGTGCAGAGCCAGCAGTACGCTGCTGTTGCCCAGCACGGCCACCGCGAA
AGTCACCGCCAGCACGGCGATCTCCAGTTTGGCCAG-3′

As a reaction solution of PCR, 5 ng of genomic DNA which is a template, mixed with each 3 μL of oligonucleotide primer solutions prepared to 5 μM, each 5 μL of 2 mM dNTPs, 5 μL of a 10× buffer (100 mM Tris-HCl pH 8.3, 500 mM KCl, 15 mM MgCl₂, 0.01% Gelatin), and 0.25 μL of 5 U/μL thermostable DNA polymerase (AmpliTaq Gold, available from ABI), and added with sterile ultrapure water to a liquid volume of 50 μL was used. The reaction solution was kept at 95° C. for 10 minutes, and then subjected to PCR conducting 40 cycles of incubation each including 30 seconds at 95° C., 30 seconds at 61° C., and 45 seconds at 72° C.

After conducting PCR, amplification was checked by 1.5% agarose gel electrophoresis, and a DNA fragment X was purified with Wizard SV Gel/PCR Kit (PROMEGA).

For a part of the obtained DNA fragment solution, a reaction solution was prepared by mixing 1 μL of SssI methylase (available from NEB), 10 μL of a 10× NEBuffer 2 (available from NEB), and 1 μL, of S-adenosyl methionine (3.2 mM, available from NEB), and adding sterile ultrapure water to a liquid volume of 100 μL. The reaction solution was incubated at 37° C. for 15 to 30 minutes, and further added with 1 μL of S-adenosyl methionine (3.2 mM, available from NEB) and incubated at 37° C. for 15 to 30 minutes. This was then purified with Wizard SV Gel/PCR Kit (PROMEGA). These operations were repeated another 5 times, to obtain a methylated DNA fragment (MX, SEQ ID NO: 27).

### <DNA Fragment> (N Denotes 5-Methylcytosine.)

(SEQ ID NO: 27)

MX: 5′-
CTCAGCACCCAGGNGGCNGNGATCATGAGGNGNGAGNGGNGNGNGGGCTG
TTGCAGAGTCTTGAGNGGGTGGCACACNGNGATGTAGNGGTNGGCTGTCA
TGACTACCAGCATGTAGGCNGANGCAAACATGCNGAACACCTGCAGGTGC
TTCACCANGNGGCACAGCCAGTNGGGGCNGNGGAAGNGGTAGGTGATGTC
CCAGCACATTTGNGGCAGCACCTGGAAGAATGCCANGGCCAGGTNGGCCA
GGCTGAGGTGTNGGATGAAGAGGTGCATGNGGGANGTCTTGNGNGGNGTC
NGGTGCAGAGCCAGCAGTANGCTGCTGTTGCCCAGCANGGCCACNGNGAA
AGTCACNGCCAGCANGGNGATCTCCAGTTTGGCCAG-3′

For each obtained DNA fragment X, the following solutions were prepared.

Solution A: 100 pg/10 μL, solution in TE

Solution B: 10 pg/10 μL, solution in TE

Solution C: 1 pg/10 μL solution in TE

Solution D: TE solution (negative control solution)

For each obtained DNA fragment MX, the following solutions were prepared.

Solution MA: 100 pg/10 μL solution in TE

Solution MB: 10 pg/10 μL, solution in TE

Solution MC: 1 pg/10 μL solution in TE

Solution MD: TE solution (negative control solution)

Counter oligonucleotides C1 to C12 comprising the nucleotide sequences of SEQ ID NO: 29 to SEQ ID NO; 40 capable of complementarily binding with a minus strand of the target DNA region comprising the nucleotide sequence of SEQ ID NO: 28 were synthesized, and each 0.01 μM solutions in TE buffer were prepared.

### <Target DNA Region> (5-Methylcytosine is Also Denoted by C.)

(SEQ ID NO: 28)

X′: 5′-
CTCAGCACCCAGGCGGCCGCGATCATGAGGCGCGAGCGGCGCGCGGGCTG
TTGCAGAGTCTTGAGCGGGTGGCACACCGCGATGTAGCGGTCGGCTGTCA
TGACTACCAGCATGTAGGCCGACGCAAACATGCCGAACACCTGCAGGTGC
TTCACCACGCGGCACAGCCAGTCGGGGCCGCGGAAGCGGTAGGTGATGTC
CCAGCACATTTGCGGCAGCACCTGGAAGAATGCCACGGCCAGGTCGGCCA
GGCTGAGGTGTCGGATGAAGAGGTGCATGCGGGACGTCTTGCGCGGCGTC
CGGTGCAGAGCCAGCAGTACGCTGCTGTTGCCCAGCACGGCCACCGCGAA
AGTCACCGCCAGCACGGCGATCTCCAGTTTGGCCAG-3′

### <Counter Oligonucleotides>

| (SEQ ID NO: 29) | |
|---|---|
| C1: | 5′-GCCACCGCGAAAGTCACCGCCAGCACGGCG-3′ |
| (SEQ ID NO: 30) | |
| C2: | 5′-GCCAGCAGTACGCTGCTGTTGCCCAGCACG-3′ |
| (SEQ ID NO: 31) | |
| C3: | 5′-CGGGACGTCTTGCGCGGCGTCCGGTGCAGA-3′ |
| (SEQ ID NO: 32) | |
| C4: | 5′-AGGCTGAGGTGTCGGATGAAGAGGTGCATG-3′ |
| (SEQ ID NO: 33) | |
| C5: | 5′-ACCTGGAAGAATGCCACGGCCAGGTCGGCC-3′ |
| (SEQ ID NO: 34) | |
| C6: | 5′-TAGGTGATGTCCCAGCACATTTGCGGCAGC-3′ |
| (SEQ ID NO: 35) | |
| C7: | 5′-CGGCACAGCCAGTCGGGGCCGCGGAAGCGG-3′ |
| (SEQ ID NO: 36) | |
| C8: | 5′-ATGCCGAACACCTGCAGGTGCTTCACCACG-3′ |
| (SEQ ID NO: 37) | |
| C9: | 5′-ATGACTACCAGCATGTAGGCCGACGCAAAC-3′ |
| (SEQ ID NO: 38) | |
| C10: | 5′-TGGCACACCGCGATGTACCGGTCGGCTGTC-3′ |
| (SEQ ID NO: 39) | |
| C11: | 5′-CGCGCGGGCTGTTGCAGAGTCTTGAGCGGG-3′ |
| (SEQ ID NO: 40) | |
| C12: | 5′-CAGGCGGCCGCGATCATGAGGCGCGAGCGG-3′ |

For each of the solutions of the DNA fragment X and the solutions of the methylated DNA fragment MX, the following treatment was executed.

To a PCR tube, 10 μL of a DNA fragment solution prepared as described above, 10 μL of a counter oligonucleotide solution prepared as described above, 5 μL of a buffer (330 mM Tris-Acetate pH 7.9, 660 mM KOAc, 100 mM MgOAc₂, 5 mM Dithiothreitol), 5 μL of a 100 mM MgCl₂ solution, and 5 μL of a 1 mg/mL BSA solution were added, and the resultant mixture was added with sterile ultrapure water to a liquid volume of 50 μL, and mixed. Thereafter, this PCR tube was heated at 95° C. for 10 minutes, rapidly cooled to 70° C., and kept at this temperature for 10 minutes. Then the tube was cooled to 50° C. and kept at this temperature for 10 minutes, and further kept at 37° C. for 10 minutes, and returned to room temperature (these correspond to First (A) step of the present measuring method).

The PCR tube coated with streptavidin to which a biotin-labeled methyl cytosine antibody was immobilized was added with 50 μL of a reaction solution of a DNA fragment prepared as described above, and left still for an hour at room temperature. Then the solution was removed by pipetting, and 100 μL of a washing buffer [0.05% Tween 20-containing phosphate buffer (1 mM KH₂PO₄, 3 mM Na₂HPO.7H₂O, 154 mM NaCl, pH 7.4)] was added, and then the buffer was removed by pipetting. This operation was repeated another two times (these correspond to First (B) step of the present measuring method).

The sample prepared as described above was added with 5 μL of a buffer (330 mM Tris-Acetate pH 7.9, 660 mM KOAc, 100 mM MgOAc₂, 5 mM Dithiothreitol), 5 μL of BSA (Bovine serum albumin 1 mg/ml), 12 U of a methylation sensitive restriction enzyme HpaII, and 5 pmol of a masking oligonucleotide M1 comprising the nucleotide sequence of SEQ ID NO: 41, capable of complementarily binding with a methylation sensitive restriction enzyme HpaII recognition site existing in a target DNA region X′ comprising the nucleotide sequence of SEQ ID NO: 28, and the resultant mixture was further added with sterile ultrapure water to a liquid volume of 50 μL.

### <Target DNA Region> (5-Methylcytosine is Also Denoted by C.)

(SEQ ID NO: 28)

X′: 5′-
CTCAGCACCCAGGCGGCCGCGATCATGAGGCGCGAGCGGCGCGCGGGCTG
TTGCAGAGTCTTGAGCGGGTGGCACACCGCGATGTAGCGGTCGGCTGTCA
TGACTACCAGCATGTAGGCCGACGCAAACATGCCGAACACCTGCAGGTGC
TTCACCACGCGGCACAGCCAGTCGGGGCCGCGGAAGCGGTAGGTGATGTC
CCAGCACATTTGCGGCAGCACCTGGAAGAATGCCACGGCCAGGTCGGCCA
GGCTGAGGTGTCGGATGAAGAGGTGCATGCGGGACGTCTTGCGCGGCGTC
CGGTGCAGAGCCAGCAGTACGCTGCTGTTGCCCAGCACGGCCACCGCGAA
AGTCACCGCCAGCACGGCGATCTCCAGTTTGGCCAG-3′

### <Masking Oligonucleotide>

| | | |
|---|---|---|
| M1: | 5′-TGCACCGGACGC-3′ | (SEQ ID NO: 41) |

After incubating each reaction solution at 37° C. for an hour, the solution was removed by pipetting, and 100 μL of a washing buffer [0.05% Tween 20-containing phosphate buffer (1 mM KH₂PO₄, 3 mM Na₂HPO.7H₂O, 154 mM NaCl, pH 7.4)] was added, and then the buffer was removed by pipetting. This operation was repeated another two times (these correspond to Second step of the present measuring method).

Then by subjecting the above PCR tube to PCR using respective solutions of oligonucleotide primers PF1 and PR1 comprising the nucleotide sequences of SEQ ID NO: 24 and SEQ ID NO: 25, and the following reaction condition, methylated DNA in a target DNA region X′ comprising the nucleotide sequence of SEQ ID NO: 28 was amplified.

### <Oligonucleotide Primers Designed for PCR>

| | | |
|---|---|---|
| PF1: | 5′-CTCAGCACCCAGGCGGCC-3′ | (SEQ ID NO: 24) |
| PR1: | 5′-CTGGCCAAACTGGAGATCGC-3′ | (SEQ ID NO: 25) |

### <Target DNA Region> (5-Methylcytosine is Also Denoted by C.)

(SEQ ID NO: 28)

X′: 5′-
CTCAGCACCCAGGCGGCCGCGATCATGAGGCGCGAGCGGCGCGCGGGCTG
TTGCAGAGTCTTGAGCGGGTGGCACACCGCGATGTAGCGGTCGGCTGTCA
TGACTACCAGCATGTAGGCCGACGCAAACATGCCGAACACCTGCAGGTGC
TTCACCACGCGGCACAGCCAGTCGGGGCCGCGGAAGCGGTAGGTGATGTC
CCAGCACATTTGCGGCAGCACCTGGAAGAATGCCACGGCCAGGTCGGCCA
GGCTGAGGTGTCGGATGAAGAGGTGCATGCGGGACGTCTTGCGCGGCGTC
CGGTGCAGAGCCAGCAGTACGCTGCTGTTGCCCAGCACGGCCACCGCGAA
AGTCACCGCCAGCACGGCGATCTCCAGTTTGGCCAG-3′

As a reaction solution of PCR, DNA which is a template, mixed with each 3 μL of oligonucleotide primer solutions prepared to 5 μM, each 5 μL of 2 mM dNTPs, 5 μL of a buffer (100 mM Tris-HCl pH 8.3, 500 mM KCl, 15 mM MgCl₂, 0.01% Gelatin), and 0.25 μL of 5 thermostable DNA polymerase (AmpliTaq Gold, available from ABI), and added with sterile ultrapure water to a liquid volume of 50 μL was used. The reaction solution was kept at 95° C. for 10 minutes, and then subjected to PCR conducting 25 cycles of incubation each including 20 seconds at 95° C., 30 seconds at 61° C., and 30 seconds at 72° C.

After conducting PCR, amplification was checked by 1.5% agarose gel electrophoresis. The result is as follows (these correspond to Third step of the present measuring method).

The result is shown in FIG. 2. In Solutions MA, MB and MC of the methylated DNA fragment MX, amplification was observed, and an amplification product thereof was obtained. In the negative control solution MD, amplification was not observed, and an amplification product was not obtained. In solutions A, B, C and D of the unmethylated DNA fragment X, amplification was not observed, and an amplification product thereof was not obtained.

From the above, it was demonstrated that DNA containing a methylated target DNA region can be selected by an immobilized methyl cytosine antibody, and amplified DNA can be detected with higher sensitivity by amplifying methylated DNA to a detectable level.

### Example 3

A commercially available methylated cytosine antibody (available from Aviva Systems Biology) was labeled with biotin using a commercially available biotinylating kit (Biotin Labeling Kit-NH₂, available from DOJINDO Laboratories) according to the method described in the catalogue. The obtained biotin-labeled methylated cytosine antibody was refrigerated as a solution [about 0.1 μg/100 μL solution of an antibody in a 0.1% BSA-containing phosphate buffer (1 mM KH₂PO₄, 3 mM Na₂HPO.7H₂O, 154 mM NaCl, pH 7.4)].

To each PCR tube coated with streptavidin (a total of 8 tubes), 50 μL of the synthetically obtained biotin-labeled methyl cytosine antibody solution was added and immobilized to the PCR tube by leaving it still for about an hour at room temperature. Then, after removing the solution by pipetting, 100 μL of a washing buffer [0.05% Tween 20-containing phosphate buffer (1 mM KH₂PO₄, 3 mM Na₂HPO.7H₂O, 154 mM NaCl, pH 7.4)] was added, and then the buffer was removed by pipetting. This operation was repeated another two times (these correspond to preparation of an immobilized methylated DNA antibody used in the present measuring method).

For genomic DNA derived from human blood purchased from Clontech, a DNA fragment (Y, SEQ ID NO: 44, a region corresponding to the base numbers 76606 to 76726 shown in Genbank Accession No. ac009800 and so on) to be used as a test sample was amplified by conducting PCR using oligonucleotide primers (PF2 and PR2) designed for PCR of SEQ ID NO: 42 and SEQ ID NO: 43 and the following reaction condition.

### <Oligonucleotide Primers Designed for PCR>

| | | |
|---|---|---|
| PF2: | 5′-TGAGCTCCGTAGGGCGTCC-3′ | (SEQ ID NO: 42) |
| PR2: | 5′-GCGCCGGGTCCGGGCCC-3′ | (SEQ ID NO: 43) |

### <DNA Fragment>

(SEQ ID NO: 44)

Y: 5′-
GCGCCGGGTCCGGGCCCGATGCGTTGGCGGGCCAGGGCTCCGAGAACGAG
GCGTTGTCCATCTCAACGAGGGCAGAGGAGCCGGCCACCTGGCGTCCCCC
AAGGACGCCCTACGGAGCTCA-3′

As a reaction solution of PCR, 5 ng of genomic DNA which is a template, mixed with each 3 μL of oligonucleotide primer solutions prepared to 5 μM, each 5 μL of 2 mM dNTPs, 5 μL of a 10× buffer (100 mM Tris-HCl pH 8.3, 500 mM KCl, 15 mM MgCl₂, 0.01% Gelatin), and 0.25 μL of 5 U/μL thermostable DNA polymerase (AmpliTaq Gold, available from ABI), and added with sterile ultrapure water to a liquid volume of 50 μL was used. The reaction solution was kept at 95° C. for 10 minutes, and then subjected to PCR conducting 50 cycles of incubation each including 30 seconds at 95° C., 30 seconds at 60° C., and 45 seconds at 72° C.

After conducting PCR, amplification was checked by 1.5% agarose gel electrophoresis, and a DNA fragment Y was purified with Wizard SV Gel/PCR Kit (PROMEGA).

For a part of the obtained DNA fragment solution, a reaction solution was prepared by mixing 1 μL of SssI methylase (available from NEB), 10 μL of a 10× NEBuffer 2 (available from NEB), and 1 μL, of S-adenosyl methionine (3.2 mM, available from NEB), and adding sterile ultrapure water to a liquid volume of 100 μL. The reaction solution was incubated at 37° C. for 15 to 30 minutes, and further added with 1 μL of S-adenosyl methionine (3.2 mM, available from NEB) and incubated at 37° C. for 15 to 30 minutes. This was then purified with Wizard SV Gel/PCR Kit (PROMEGA). These operations were repeated another 5 times, to obtain a methylated DNA fragment (MY, SEQ ID NO; 45).

### <DNA Fragment> (N Denotes 5-Methylcytosine.)

(SEQ ID NO: 45)

MY: 5′-
GNGCNGGGTCNGGGCCNGATGNGTTGGNGGGCCAGGGCTCNGAGAANGAG
GNGTTGTCCATCTCAANGAGGGCAGAGGACCNGGNGACCTGGNGTCCCCC
AAGGANGCCCTANGGAGCTCA-3′

For each obtained DNA fragment Y, the following solutions were prepared.

Solution A: 100 pg/10 μL solution in TE

Solution B: 10 pg/10 μL solution in TE

Solution C: 1 pg/10 μL solution in TE

Solution D: TE solution (negative control solution)

For each obtained DNA fragment MY, the following solutions were prepared.

Solution MA: 100 pg/10 μL solution in TE

Solution MB: 10 pg/10 μL solution in TE

Solution MC: 1 pg/10 μL solution in TE

Solution MD: TE solution (negative control solution)

Counter oligonucleotides C13, C14, and C15 comprising the nucleotide sequences of SEQ ID NO: 47, SEQ ID NO: 25, and SEQ ID NO: 49 capable of complementarily binding with a minus strand of the target DNA region Y′ comprising the nucleotide sequence of SEQ ID NO: 46 were synthesized, and each 0.01 μM solutions in a TE buffer were prepared.

### <Target DNA Region> (5-Methylcytosine is Also Denoted by C.)

(SEQ ID NO: 46)

Y′: 5′-
GCGCCGGGTCCGGGCCCGATGCGTTGGCGGGCCAGGGCTCCGAGAACGAG
GCGTTGTCCATCTCAACGAGGGCAGAGGAGCCGGCGACCTGGCGTCCCCC
AAGGACGCCCTACGGAGCTCA-3′

### <Counter Oligonucleotides>

| (SEQ ID NO: 47) | |
|---|---|
| C13: | 5′-GCGTCCCCCAAGGACGCCCTACGGAGCTCA-3′ |
| (SEQ ID NO: 48) | |
| C14: | 5′-CTCAACGAGGGCAGAGGAGCCGGCGACCTG-3′ |
| (SEQ ID NO: 49) | |
| C15: | 5′-CGCCGGGTCCGGGCCCGATGCGTTGGCGGG-3′ |

For each of the solutions of the DNA fragment Y and the solutions of the methylated DNA fragment MY, the following treatment was executed.

To a PCR tube, 10 μL of a DNA fragment solution prepared as described above, 10 μL of a counter oligonucleotide solution prepared as described above, 5 μL of a buffer (330 mM Tris-Acetate pH 7.9, 660 mM KOAc, 100 mM MgOAc₂, 5 mM Dithiothreitol), 5 μL of a 100 mM MgCl₂ solution, and 5 μL of a 1 mg/mL BSA solution were added, and the resultant mixture was added with sterile ultrapure water to a liquid volume of 50 μL, and mixed. Thereafter, this PCR tube was heated at 95° C. for 10 minutes, rapidly cooled to 70° C., and kept at this temperature for 10 minutes. Then the tube was cooled to 50° C. and kept at this temperature for 10 minutes, and further kept at 37° C. for 10 minutes, and returned to room temperature (these correspond to First (A) step of the present measuring method).

The PCR tube coated with streptavidin to which a biotin-labeled methyl cytosine antibody was immobilized was added with 50 μL of a reaction solution of a DNA fragment prepared as described above, and left still for an hour at room temperature. Then the solution was removed by pipetting, and 100 μL of a washing buffer [0.05% Tween 20-containing phosphate buffer (1 mM KH₂PO₄, 3 mM Na₂HPO.7H₂O, 154 mM NaCl, pH 7.4)] was added, and then the buffer was removed by pipetting. This operation was repeated another two times (these correspond to First (B) step of the present measuring method).

The sample prepared as described above was added with 5 μL of a buffer (330 mM Tris-Acetate pH 7.9, 660 mM KOAc, 100 mM MgOAc₂, 5 mM Dithiothreitol), 5 μL of BSA (Bovine serum albumin 1 mg/ml), 12 U of a methylation sensitive restriction enzyme HpaII, and 5 pmol of a masking oligonucleotide M2 comprising the nucleotide sequence of SEQ ID NO: 50, capable of complementarily binding with a methylation sensitive restriction enzyme HpaII recognition site existing in a target DNA region Y′ comprising the nucleotide sequence of SEQ ID NO: 46, and the resultant mixture was further added with sterile ultrapure water to a liquid volume of 50 μL.

### <Target DNA Region>

(SEQ ID NO: 46)

Y′: 5′-
GCGCGGGGTCCGGGCCCGATGCGTTGGCGGGCCAGGGCTCCGAGAACGAG
GCGTTGTCCATCTCAACGAGGGCAGAGGAGCCGGCGACCTGGCGTCCCCC
AAGGACGCCCTACGGAGCTCA-3′

### <Masking Oligonucleotide>

| | | |
|---|---|---|
| M2: | 5′-GTCGCCGGCTCC-3′ | (SEQ ID NO: 50) |

After incubating each reaction solution at 37° C. for an hour, the solution was removed by pipetting, and 100 μL of a washing buffer [0.05% Tween 20-containing phosphate buffer (1 mM KH₂PO₄, 3 mM Na₂HPO.7H₂O, 154 mM NaCl, pH 7.4)] was added, and then the buffer was removed by pipetting. This operation was repeated another two times (these correspond to Second step of the present measuring method).

Then by subjecting the above PCR tube to PCR using respective solutions of oligonucleotide primers PF2 and PR2 comprising the nucleotide sequences of SEQ ID NO: 42 and SEQ ID NO: 43, and the following reaction condition, methylated DNA in a target DNA region Y′ comprising the nucleotide sequence of SEQ ID NO: 46 was amplified.

### <Oligonucleotide Primers Designed for PCR>

| | | |
|---|---|---|
| PF2: | 5′-TGAGCTCCGTAGGGCGTCC-3′ | (SEQ ID NO: 42) |
| PR2: | 5′-GCGCCGGGTCCGGGCCC-3′ | (SEQ ID NO: 43) |

### <Target DNA Region>

(SEQ ID NO: 46)

Y′: 5′-
GCGCCGGGTCCGGGCCCGATGCGTTGGCGGGCCAGGGCTCCGAGAACGAG
GCGTTGTCCATCTCAACGAGGGCAGAGGAGCCGGCGACCTGGCGTCCCCC
AAGGACGCCCTACGGAGCTCA-3′

As a reaction solution of PCR, DNA which is a template, mixed with each 3 μL of oligonucleotide primer solutions prepared to 5 μM, each 5 μL of 2 mM dNTPs, 5 μL of a 10× buffer (100 mM Tris-HCl pH 8.3, 500 mM KCl, 15 mM MgCl₂, 0.01% Gelatin), and 0.25 μL of 5 U/μL thermostable DNA polymerase (AmpliTaq Gold, available from ABI), and added with sterile ultrapure water to a liquid volume of 50 μL was used. The reaction solution was kept at 95° C. for 10 minutes, and then subjected to PCR conducting 25 cycles of incubation each including 20 seconds at 95° C., 30 seconds at 60° C., and 30 seconds at 72° C.

After conducting PCR, amplification was checked by 1.5% agarose gel electrophoresis. The result is as follows (these correspond to Third step of the present measuring method).

The result is shown in FIG. 3. In Solutions MA, MB and MC of the methylated DNA fragment MY, amplification was observed, and an amplification product thereof was obtained. In the negative control solution MD, amplification was not observed, and an amplification product was not obtained. In solutions A, B, C and D of the unmethylated DNA fragment Y, amplification was not observed, and an amplification product thereof was not obtained.

From the above, it was demonstrated that DNA containing a methylated target DNA region can be selected by an immobilized methyl cytosine antibody, and amplified DNA can be detected with higher sensitivity by amplifying methylated DNA to a detectable level.

### Example 4

A commercially available methylated cytosine antibody (available from Aviva Systems Biology) was labeled with biotin using a commercially available biotinylating kit (Biotin Labeling Kit-NH₂, available from DOJINDO Laboratories) according to the method described in the catalogue. The obtained biotin-labeled methylated cytosine antibody was refrigerated as a solution [about 0.1 μg/100 μL, solution of an antibody in a 0.1% BSA-containing phosphate buffer (1 mM KH₂PO₄, 3 mM Na₂HPO.7H₂O, 154 mM NaCl, pH 7.4)].

To each PCR tube coated with streptavidin (a total of 8 tubes), 50 μL of the synthetically obtained biotin-labeled methyl cytosine antibody solution was added and immobilized to the PCR tube by leaving it still for about an hour at room temperature. Then, after removing the solution by pipetting, 100 μL of a washing buffer [0.05% Tween 20-containing phosphate buffer (1 mM KH₂PO₄, 3 mM Na₂HPO.7H₂O, 154 mM NaCl, pH 7.4)] was added, and then the buffer was removed by pipetting. This operation was repeated another two times (these correspond to preparation of an immobilized methylated DNA antibody used in the present measuring method).

Yeast strain X2180-1A of baker's yeast was cultured in a YPD medium (1% Yeast extract, 2% Peptone, 2% Glucose, pH 5.6 to 6.0) to a turbidity of OD₆₀₀ 0.6 to 1.0, and centrifuged at 10,000 g for 10 minutes, to prepare 1×10⁷ of yeast cells. From the prepared yeast cells, a yeast genome was acquired using a generally used preparation method of a yeast genome as described in Methods in Yeast Genetics (Cold Spring Harbor Laboratory).

The prepared yeast cells were suspended in Buffer A (1 M sorbitol, 0.1 M EDTA, pH 7.4), added with 2-mercaptoethanol (final concentration 14 mM) and 100 U zymolase (10 mg/ml), and incubated under stirring at 30° C. for an hour until the solution became clear. After collecting a protoplast by centrifugation at 550 g for 10 minutes, it was suspended in Buffer B (50 mM Tris-HCl, pH 7.4, 20 mM EDTA), added with sodium dodecyl sulfate in 1% (w/v), and then incubated at 65° C. for 30 minutes. Sequentially, 5 M CH₃COOK was added and mingled in a volume ratio of 2/5, and the mixture was cooled on ice for 30 minutes, and then centrifuged at 15,000 g for 30 minutes to collect the supernatant. The collected supernatant was added with 3 M CH₃COONa in a volume ratio of 1/10 and an equal amount of isopropanol and mingled well, and the precipitate obtained by centrifugation at 15,000 g at 4° C. for 30 minutes was rinsed with 70% ethanol and collected. After drying, the precipitate was dissolved in 1 mL of TE buffer (10 mM Tris-HCl, pH 8.0, 1 mM EDTA), and added with RNase A (available from Sigma) in a concentration of 40 μg/ml, incubated at 37° C. for an hour, and then the mixture was added with proteinase K (available from Sigma) and sodium dodecyl sulfate in a concentrations of 500 μg/mL and 1% (w/v), respectively, and shaken at 55° C. for about 16 hours. After end of the shaking, the mixture was extracted with phenol [saturated with 1 M Tris-HCl (pH 8.0)].chloroform. An aqueous layer was collected, added with NaCl in a concentration of 0.5 N, and allowed to precipitate from ethanol, and the generated precipitate was collected. The collected precipitate was rinsed with 70% ethanol, to obtain genomic DNA.

From the obtained genomic DNA, a DNA fragment to be used as a test sample (Z, SEQ ID NO: 53, a region corresponding to the base numbers 384569 to 384685 of yeast chromosome VII shown in Genbank Accession No. NC_{—}001139 and so on) was amplified by conducting PCR using oligonucleotide primers (PF3 and PR3) designed for PCR of SEQ ID NO: 51 and SEQ ID NO: 29 and the following reaction condition.

### <Oligonucleotide Primers Designed for PCR>

| | | |
|---|---|---|
| PF3: | 5′-GGACCTGTGTTTGACGGGTAT-3′ | (SEQ ID NO: 51) |
| PR3: | 5′-AGTACAGATCTGGCGTTCTCG-3′ | (SEQ ID NO: 52) |

### <DNA Fragment>

(SEQ ID NO: 53)

Z: 5′-
GGACCTGTGTTTGACGGGTATAACACTAAGTTGCGCAATTTGCTGTATTG
CGAAATCCGCCCGGACGATATCACTCTTGAGCGCATGTGCCGTTTCCGAG
AACGCCAGATCTGTACT-3′

As a reaction solution of PCR, 10 ng of genomic DNA which is a template, mixed with each 3 μL, of oligonucleotide primer solutions prepared to 5 μM, each 5 μL of 2 mM dNTPs, 5 μL of a 10× buffer (100 mM Tris-HCl pH 8.3, 500 mM KCl, 15 mM MgCl₂, 0.01% Gelatin), and 0.25 μL, of 5 U/μL thermostable DNA polymerase (AmpliTaq Gold, available from ABI), and added with sterile ultrapure water to a liquid volume of 50 μL was used. The reaction solution was kept at 95° C. for 10 minutes, and then subjected to PCR conducting 40 cycles of incubation each including 20 seconds at 95° C., 30 seconds at 58° C., and 30 seconds at 72° C.

After conducting PCR, amplification was checked by 1.5% agarose gel electrophoresis, and a DNA fragment Z was purified with Wizard SV Gel/PCR Kit (PROMEGA).

For a part of the obtained DNA fragment solution, a reaction solution was prepared by mixing 1 μL of SssI methylase (available from NEB), 10 μL of a 10× NEBuffer 2 (available from NEB), and 1 μL of S-adenosyl methionine (3.2 mM, available from NEB), and adding sterile ultrapure water to a liquid volume of 100 μL. The reaction solution was incubated at 37° C. for 15 to 30 minutes, and further added with 1 μL of S-adenosyl methionine (3.2 mM, available from NEB) and incubated at 37° C. for 15 to 30 minutes. This was then purified with Wizard SV Gel/PCR Kit (PROMEGA). These operations were repeated another 5 times, to obtain a methylated DNA fragment (MZ, SEQ ID NO: 54).

### <DNA Fragment> (N Denotes 5-Methylcytosine.)

(SEQ TO NO: 54)

MZ: 5′-
GGACCTGTGTTTGANGGGTATAACACTAAGTTGNGCAATTTGCTGTATTG
NGAAATCNGCCNGGANGATATCACTCTTGAGNGCATGTGCNGTTTCNGAG
AAGCCAGATCTGTACT-3′

For each obtained DNA fragment Z, the following solutions were prepared.

Solution A: 10 pg/10 μL, solution in TE

Solution B: 1 pg/10 μL solution in TE

Solution C: TE solution (negative control solution)

For each obtained DNA fragment MZ, the following solutions were prepared.

Solution MA: 10 pg/10 μL, solution in TE

Solution MB: 1 pg/10 μL solution in TE

Solution MC: TE solution (negative control solution)

Counter oligonucleotides C16, C17, C18 and C19 comprising the nucleotide sequences of SEQ ID NO: 56, SEQ ID NO: 57, SEQ ID NO: 58, and SEQ ID NO: 59 capable of complementarily binding with a minus strand of the target DNA region Z′ comprising the nucleotide sequence of SEQ ID NO: 55 were synthesized, and each 0.01 μM solutions in a TE buffer were prepared.

### <Target DNA Region> (Methyl Cytosine is Also Denoted by C.)

(SEQ ID NO: 55)

Z′: 5′-
GGACCTGTGTTTGACGGGTATAACACTAAGTTGCGCAATTTGCTGTATTG
CGAAATCCGCCCGGACGATATCACTCTTGAGCGCATGTGCCGTTTCCGAG
AACGCCAGATCTGTACT-3′

### <Counter Oligonucleotides>

| | |
|---|---|
| C16: | |
| 5′-AACACTAAGTTGCGCAATTTGCTGT-3′ | (SEQ ID NO; 56) |
| C17: | |
| 5′-ATTGCGAAATCCGCCCGGACGATAT-3′ | (SEQ ID NO: 57) |
| C18: | |
| 5′-CACTCTTGAGCGCATGTGCCGTTTC-3′ | (SEQ ID NO: 58) |
| C19: | |
| 5′-GGACCTGTGTTTGACGGGTAT-3′ | (SEQ ID NO: 59) |

For each of the solutions of the DNA fragment Z and the solutions of the methylated DNA fragment MZ, the following treatment was executed.

To a PCR tube, 10 μL of a DNA fragment solution prepared as described above, 10 μL of a counter oligonucleotide solution prepared as described above, 5 μL of a buffer (330 mM Tris-Acetate pH 7.9, 660 mM KOAc, 100 mM MgOAc₂, 5 mM Dithiothreitol), 5 μL of a 100 mM MgCl₂ solution, and 5 μL of a 1 mg/mL BSA solution were added, and the resultant mixture was added with sterile ultrapure water to a liquid volume of 50 μL, and mixed. Thereafter, this PCR tube was heated at 95° C. for 10 minutes, rapidly cooled to 70° C., and kept at this temperature for 10 minutes. Then the tube was cooled to 50° C. and kept at this temperature for 10 minutes, and further kept at 37° C. for 10 minutes, and returned to room temperature (these correspond to First (A) step of the present measuring method).

The PCR tube coated with streptavidin to which a biotin-labeled methyl cytosine antibody was immobilized was added with 50 μL, of a reaction solution of a DNA fragment prepared as described above, and left still for an hour at room temperature. Then the solution was removed by pipetting, and 100 μL of a washing buffer [0.05% Tween 20-containing phosphate buffer (1 mM KH₂PO₄, 3 mM Na₂HPO.7H₂O, 154 mM NaCl, pH 7.4)] was added, and then the buffer was removed by pipetting. This operation was repeated another two times (these correspond to First (B) step of the present measuring method).

The sample prepared as described above was added with 5 μL of a buffer (330 mM Tris-Acetate pH 7.9, 660 mM KOAc, 100 mM MgOAc₂, 5 mM Dithiothreitol), 5 μL of BSA (Bovine serum albumin 1 mg/ml), 12 U of a methylation sensitive restriction enzyme HpaII, and 5 pmol of a masking oligonucleotide M3 comprising the nucleotide sequence of SEQ ID NO: 60, capable of complementarily binding with a methylation sensitive restriction enzyme HpaII recognition site existing in a target DNA region Z′ comprising the nucleotide sequence of SEQ ID NO: 55, and the resultant mixture was further added with sterile ultrapure water to a liquid volume of 50 μL.

### <Target DNA Region> (Also Methyl Cytosine is Denoted by C.)

(SEQ ID NO: 55)

Z′: 5′-
GGACCTGTGTTTGACGGGTATAACACTAAGTTGCGCAATTTGCTGTATTG
CGAAATCCGCCCGGACGATATCACTCTTGAGCGCATGTGCCGTTTCCGAG
AACGCCAGATCTGTAGT-3′

### <Masking Oligonucleotide>

| | | |
|---|---|---|
| M3: | 5′-TCGTCCGGGCGG-3′ | (SEQ ID NO: 60) |

After incubating each reaction solution at 37° C. for an hour, the solution was removed by pipetting, and 100 μL of a washing buffer [0.05% Tween 20-containing phosphate buffer (1 mM KH₂PO₄, 3 mM Na₂HPO.7H₂O, 154 mM NaCl, pH 7.4)] was added, and then the buffer was removed by pipetting. This operation was repeated another two times (these correspond to Second step of the present measuring method).

Then the above PCR tube was subjected to PCR using respective solutions of oligonucleotide primers PF3 and PR3 comprising the nucleotide sequences of SEQ ID NO: 51 and SEQ ID NO: 52, and the following reaction condition, methylated DNA in a target DNA region comprising the nucleotide sequence of SEQ ID NO: 55 was amplified.

### <Oligonucleotide Primers Designed for PCR>

| | | |
|---|---|---|
| PF3: | 5′-GGACCTGTGTTTGACGGGTAT-3′ | (SEQ ID NO: 51) |
| PR3: | 5′-AGTACAGATCTGGCGTTCTCG-3′ | (SEQ ID NO: 52) |

### <Target DNA Region> (Also 5-Methylcytosine is Denoted by C.)

(SEQ ID NO: 55)

Z′: 5′-
GGACCTGTGTTTGACGGGTATAACACTAAGTTGCGCAATTTGCTGTATTG
CGAAATCCGCCCGGACGATATCACTCTTGAGCGCATGTGCCGTTTCCGAG
AACGCCAGATCTGTACT-3′

As a reaction solution of PCR, DNA which is a template, mixed with each 3 μL of oligonucleotide primer solutions prepared to 5 μM, each 5 μL, of 2 mM dNTPs, 5 of a buffer (100 mM Tris-HCl pH 8.3, 500 mM KCl, 15 mM MgCl₂, 0.01% Gelatin), and 0.25 μL of 5 U/μL thermostable DNA polymerase (AmpliTaq Gold, available from ABI), and added with sterile ultrapure water to a liquid volume of 50 μL was used. The reaction solution was kept at 95° C. for 10 minutes, and then subjected to PCR conducting 28 cycles of incubation each including 20 seconds at 95° C., 30 seconds at 58° C., and 30 seconds at 72° C.

After conducting PCR, amplification was checked by 1.5% agarose gel electrophoresis. The result is as follows (these correspond to Third step of the present measuring method).

The result is shown in FIG. 4. In Solutions MA and MB of the methylated DNA fragment MZ, amplification was observed, and an amplification product thereof was obtained. In the negative control solution MC, amplification was not observed, and an amplification product was not obtained. In solutions A, B and C of the unmethylated DNA fragment Z, amplification was not observed, and an amplification product thereof was not obtained.

From the above, it was demonstrated that DNA containing a methylated target DNA region can be selected by an immobilized methyl cytosine antibody, and amplified DNA can be detected with higher sensitivity by amplifying methylated DNA to a detectable level.

### Example 5

A commercially available methylated cytosine antibody (available from Aviva Systems Biology) was labeled with biotin using a commercially available biotinylating kit-(Biotin Labeling Kit-NH₂, available from DOJINDO Laboratories) according to the method described in the catalogue. The obtained biotin-labeled methylated cytosine antibody was refrigerated as a solution [about 0.1 μg/100 μL solution of an antibody in a 0.1% BSA-containing phosphate buffer (1 mM KH₂PO₄, 3 mM Na₂HPO.7H₂O, 154 mM NaCl, pH 7.4)].

To each PCR tube coated with streptavidin (a total of 8 tubes), 50 μL of the synthetically obtained biotin-labeled methyl cytosine antibody solution was added and immobilized to the PCR tube by leaving it still for about an hour at room temperature. Then, after removing the solution by pipetting, 100 μL of a washing buffer [0.05% Tween 20-containing phosphate buffer (1 mM KH₂PO₄, 3 mM Na₂HPO.7H₂O, 154 mM NaCl, pH 7.4)] was added, and then the buffer was removed by pipetting. This operation was repeated another two times (these correspond to preparation of an immobilized methylated DNA antibody used in the present measuring method).

Yeast strain X2180-1A of baker's yeast was cultured in a YPD medium (1% Yeast extract, 2% Peptone, 2% Glucose, pH 5.6 to 6.0) to a turbidity of OD₆₀₀ 0.6 to 1.0, and centrifuged at 10,000 g for 10 minutes, to prepare 1×10⁷ of yeast cells. From the prepared yeast cells, a yeast genome was acquired using a generally used preparation method of a yeast genome as described in Methods in Yeast Genetics (Cold Spring Harbor Laboratory).

The prepared yeast cells were suspended in Buffer A (1 M sorbitol, 0.1 M EDTA, pH 7.4), added with 2-mercaptoethanol (final concentration 14 mM) and 100 U zymolase (10 mg/ml), and incubated under stirring at 30° C. for an hour until the solution became clear. After collecting a protoplast by centrifugation at 550 g for 10 minutes, it was suspended in Buffer B (50 mM Tris-HCl, pH 7.4, 20 mM EDTA), added with sodium dodecyl sulfate in 1% (w/v), and then incubated at 65° C. for 30 minutes. Sequentially, 5 M CH₃COOK was added and mingled in a volume ratio of 2/5, and the mixture was cooled on ice for 30 minutes, and then centrifuged at 15,000 g for 30 minutes to collect the supernatant. The collected supernatant was added with 3 M CH₃COONa in a volume ratio of 1/10 and an equal amount of isopropanol and mingled well, and the precipitate obtained by centrifugation at 15,000 g at 4° C. for 30 minutes was rinsed with 70% ethanol and collected. After drying, the precipitate was dissolved in 1 mL of TE buffer (10 mM Tris-HCl, pH 8.0, 1 mM EDTA), and added with RNase A (available from Sigma) in a concentration of 40 μg/ml, incubated at 37° C. for an hour, and then the mixture was added with proteinase K (available from Sigma) and sodium dodecyl sulfate in a concentrations of 500 μg/mL and 1% (w/v), respectively, and shaken at 55° C. for about 16 hours. After end of the shaking, the mixture was extracted with phenol [saturated with 1 M Tris-HCl (pH 8.0)].chloroform. An aqueous layer was collected, added with NaCl in a concentration of 0.5 N, and allowed to precipitate from ethanol, and the generated precipitate was collected. The collected precipitate was rinsed with 70% ethanol, to obtain genomic DNA.

Part of the obtained genomic DNA was mixed with 1 μL of SssI methylase (available from NEB), 10 μL of a 10× NEBuffer 2 (available from NEB), and 1 μL of S-adenosyl methionine (3.2 mM, available from NEB), and added with sterile ultrapure water to a liquid volume of 100 μL. The reaction solution was incubated at 37° C. for 15 to 30 minutes, added with 1 μL of S-adenosyl methionine (3.2 mM, available NEB) and incubated at 37° C. for 15 to 30 minutes. This was then purified by Wizard SV Gel/PCR Kit (PROMEGA). This operation was repeated three times, and methylated genomic DNA was obtained.

For the obtained genomic DNA, the following solutions were prepared.

Solution A: 10 ng/5 μL solution in TE

Solution B: 1 ng/5 μL solution in TE

Solution C, 0.1 ng/5 μL solution in TE

Solution D: TE solution (negative control solution)

For the obtained methylated genomic DNA, the following solutions were prepared.

Solution MA: 10 ng/5 μL solution in TE

Solution MB: 1 ng/5 μL solution in TE

Solution MC: 0.1 ng/5 μL, solution in TE

Solution MD: TE solution (negative control solution)

For each of the genomic DNA solutions and methylated genomic DNA solutions prepared as described above, the following treatment was executed.

A PCR tube was added with 5 μL of the solution prepared as described above, 10 U of a restriction enzyme XspI, and 2 μL of a 10× buffer (200 mM Tris-HCl pH 8.5, 100 mM MgCl₂, 10 mM Dithiothreitol, 1000 mM KCl) suited for XspI, and added with sterile ultrapure water to a liquid volume of 20 μL. The reaction solution was incubated at 37° C. for an hour.

Counter oligonucleotides C16, C17, C18, C19, C20, C21, C22, and C23 comprising the nucleotide sequences of SEQ ID NO: 56, SEQ ID NO: 57, SEQ ID NO: 58, SEQ ID NO: 59, SEQ ID NO: 62, SEQ ID NO: 63, SEQ ID NO: 64, and SEQ ID NO: 65, capable of complementarily binding with a minus strand of the target DNA region (S, a region corresponding to the base numbers 384523 to 384766 of yeast chromosome VII shown in Genbank Accession No. NC_{—}001139 and so on) comprising the nucleotide sequence of SEQ ID NO: 61 were synthesized, and each 0.01 μM solutions in a TE buffer were prepared.

### <Target DNA Region> (Also Methyl Cytosine is Denoted by C.)

(SEQ ID NO: 61)

S: 5′-
TAGGAAATACATTCCGAGGGCGCCCGCACAAGGCCTATTATTAGAGGGAC
CTGTGTTTGACGGGTATAACACTAAGTTGCGCAATTTGCTGTATTGCGAA
ATCCGCCCGGACGATATCACTCTTGAGCGCATGTGCCGTTTCCGAGAACG
CCAGATCTGTACTGCGATCGCACACGAGGAGACACAGCGTCACGTGTTTT
GCCATTTTGTACGACAAATGAACCGCCTGGCCACGCCTCTAATC-3′

### <Counter Oligonucleotides>

| (SEQ ID NO; 56) | |
|---|---|
| C16: | 5′-AACACTAAGTTGCGCAATTTGCTGT-3′ |
| (SEQ ID NO: 57) | |
| C17: | 5′-ATTGCGAAATCCGCCCGGACGATAT-3′ |
| (SEQ ID NO: 58) | |
| C18: | 5′-CACTCTTGAGCGCATGTGCCGTTTC-3′ |
| (SEQ ID NO: 59) | |
| C19: | 5′-GGACCTGTGTTTGACGGGTAT-3′ |
| (SEQ ID NO: 62) | |
| C20: | 5′-AATACATTCCGAGGGCGCCCGCACAAGGCC-3′ |
| (SEQ ID NO: 63) | |
| C21: | 5′-GCGATCGCACACGAGGAGACA-3′ |
| (SEQ ID NO: 64) | |
| C22: | 5′-AGCGTCACGTGTTTTGCCATTTTGTACGAC-3′ |
| (SEQ ID NO: 65) | |
| C23: | 5′-AAATGAACCGCCTGGCCACGCCTCTAATC-3′ |

For each of the reaction solution of genomic DNA and methylated genomic DNA, the following treatment was executed.

To a PCR tube, 20 μL, of a reaction solution prepared as described above, 10 μL of a counter oligonucleotide solution prepared as described above, 5 μL of a buffer (330 mM Tris-Acetate pH 7.9, 660 mM KOAc, 100 mM MgOAc₂, 5 mM Dithiothreitol), 5 μL of a 100 mM MgCl₂ solution, and 5 μL of a 1 mg/mL BSA solution were added, and the resultant mixture was added with sterile ultrapure water to a liquid volume of 50 μL, and mixed. Thereafter, this PCR tube was heated at 95° C. for 10 minutes, rapidly cooled to 70° C., and kept at this temperature for 10 minutes. Then the tube was cooled to 50° C. and kept at this temperature for 10 minutes, and further kept at 37° C. for 10 minutes, and returned to room temperature (these correspond to First (A) step of the present measuring method).

The PCR tube coated with streptavidin to which a biotin-labeled methyl cytosine antibody was immobilized was added with 50 μL of a reaction solution of a DNA fragment prepared as described above, and left still for an hour at room temperature. Then the solution was removed by pipetting, and 100 μL of a washing buffer [0.05% Tween 20-containing phosphate buffer (1 mM KH₂PO₄, 3 mM Na₂HPO.7H₂O, 154 mM NaCl, pH 7.4)] was added, and then the buffer was removed by pipetting. This operation was repeated another two times (these correspond to First (B) step of the present measuring method).

The sample prepared as described above was added with 5 μL of a buffer (330 mM Tris-Acetate pH 7.9, 660 mM KOAc, 100 mM MgOA_{c2}, 5 mM Dithiothreitol), 5 μL of BSA (Bovine serum albumin 1 mg/ml), 12 U of a methylation sensitive restriction enzyme HpaII, and 5 pmol of a masking oligonucleotide M3 comprising the nucleotide sequence of SEQ ID NO: 60, capable of complementarily binding with a methylation sensitive restriction enzyme HpaII recognition site existing in a target DNA region S comprising the nucleotide sequence of SEQ ID NO: 61, and the resultant mixture was further added with sterile ultrapure water to a liquid volume of 50 μL.

### <Target DNA Region> (Also Methyl Cytosine is Denoted by C.)

(SEQ TD NO: 61)

S: 5′-
TAGGAAATACATTCCGAGGGCGCCCGCACAAGGCCTATTATTAGAGGGAC
CTGTGTTTGACGGGTATAACACTAAGTTGCGCAATTTGCTGTATTGCGAA
ATCCGCCCGGACGATATCACTCTTGAGCGCATGTGCCGTTTCCGAGAACG
CCAGATCTGTACTGCGATCGCACACGAGGAGACACAGCGTCACGTGTTTT
GCCATTTTGTACGACAAATGAACCGCCTGGCCACGCCTCTAATC-3′

### <Masking Oligonucleotide>

| | | |
|---|---|---|
| M3: | 5′-TCGTCCGGGCGG-3′ | (SEQ ID NO: 60) |

After incubating each reaction solution at 37° C. for an hour, the solution was removed by pipetting, and 100 μL, of a washing buffer [0.05% Tween 20-containing phosphate buffer (1 mM KH₂PO₄, 3 mM Na₂HPO.7H₂O, 154 mM NaCl, pH 7.4)] was added, and then the buffer was removed by pipetting. This operation was repeated another two times (these correspond to Second step of the present measuring method).

Then by subjecting the above PCR tube to PCR using respective solutions of oligonucleotide primers PF3 and PR3 comprising the nucleotide sequences of SEQ ID NO: 51 and SEQ ID NO: 52, and the following reaction condition, methylated DNA in a target DNA region Z′ comprising the nucleotide sequence of SEQ ID NO: 55 which is a partial sequence of the target DNA region S comprising the nucleotide sequence of SEQ ID: 61 was amplified.

### <Oligonucleotide Primers Designed for PCR>

| | | |
|---|---|---|
| PF3: | 5′-GGACCTGTGTTTGACGGGTAT-3′ | (SEQ ID NO: 51) |
| PR3: | 5′-AGTACAGATCTGGCGTTCTCG-3′ | (SEQ ID NO: 52) |

### <Target DNA Region> (Also Methyl Cytosine is Denoted by C.)

(SEQ ID NO: 61)

S: 5′-
TAGGAAATACATTCCGAGGGCGCCCGCACAAGGCCTATTATTAGAGGGAC
CTGTGTTTGACGGGTATAACACTAAGTTGCGCAATTTGCTGTATTGCGAA
ATCCGCCCGGACGATATCACTCTTGAGCGCATGTGCCGTTTCCGAGAACG
CCAGATCTGTACTGCGATCGCACACGAGGAGACACAGCGTCACGTGTTTT
GCCATTTTGTACGACAAATGAACCGCCTGGCCACGCCTCTAATC-3′

### <Target DNA Region> (Also 5-Methylcytosine is Denoted by C.)

(SEQ ID NO: 55)

Z′: 5′-
GGACCTGTGTTTGACGGGTATAACACTAAGTTGCGCAATTTGCTGTATTG
CGAAATCCGCCCGGACGATATCACTCTTGAGCGCATGTGCCGTTTCCGAG
AACGCCAGATCTGTACT-3′

As a reaction solution of PCR, DNA which is a template, mixed with each 3 μL of oligonucleotide primer solutions prepared to 5 μM, each 5 μL of 2 mM dNTPs, 5 μL of a buffer (100 mM Tris-HCl pH 8.3, 500 mM KCl, 15 mM MgCl₂, 0.01% Gelatin), and 0.25 μL of 5 U/μL thermostable DNA polymerase (AmpliTaq Gold, available from ABI), and added with sterile ultrapure water to a liquid volume of 50 μL was used. The reaction solution was kept at 95° C. for 10 minutes, and then subjected to PCR conducting 31 cycles of incubation each including 20 seconds at 95° C., 30 seconds at 58° C., and 30 seconds at 72° C.

After conducting PCR, amplification was checked by 1.5% agarose gel electrophoresis. The result is as follows (these correspond to Third step of the present measuring method).

The result is shown in FIG. 5. In Solutions MA, MB and MC of methylated genomic DNA, amplification was observed, and an amplification product thereof was obtained. In the negative control solution MD, amplification was not observed, and an amplification product was not obtained. In solutions A, B, C and D of unmethylated genomic DNA, amplification was not observed, and an amplification product thereof was not obtained.

From the above, it was demonstrated that single-stranded DNA containing a methylated target DNA region can be selected by an immobilized methylated cytosine antibody, and a methylation sensitive restriction enzyme recognition site protected by a masking oligonucleotide can digest an unmethylated target DNA region by a treatment with a methylation sensitive restriction enzyme after addition and mixing of a masking oligonucleotide, and only methylated DNA can be amplified to a detectable level and an amount of amplified DNA can be quantified while unmethylated DNA in the target DNA region is not amplified.

From the above, it was demonstrated that DNA containing a methylated target DNA region can be selected by an immobilized methyl cytosine antibody, and methylated DNA is amplified to a detectable level and the amplified DNA can be detected with higher sensitivity.

### INDUSTRIAL APPLICABILITY

Based on the present invention, it becomes possible to provide a method of measuring the content of methylated DNA in an objective DNA region in a genomic DNA contained in a biological specimen in a simple and convenient manner, and so on.

### Free Text in Sequence Listing

### SEQ ID NO:17

### Designed Oligonucleotide

### SEQ ID NO:18

### Designed Oligonucleotide

### SEQ ID NO:19

### Designed Oligonucleotide

### SEQ ID NO:20

### Designed Biotinated Oligonucleotide

### SEQ ID NO:21

### Designed Oligonucleotide Primer for PCR

### SEQ ID NO:22

### Designed Oligonucleotide Primer for PCR

### SEQ ID NO:23

### Designed Oligonucleotide

### SEQ ID NO:24

### Designed Oligonucleotide Primer for PCR

### SEQ ID NO:25

### Designed Oligonucleotide Primer for PCR

### SEQ ID NO:26

### Designed Oligonucleotide Consist of Objective DNA Domain

### SEQ ID NO:27

### Designed Oligonucleotide Consist of Objective DNA Domain

### SEQ ID NO:28

### Designed Oligonucleotide Consist of Objective DNA Domain

### SEQ ID NO:29

### Designed Oligonucleotide for Experiment

### SEQ ID NO:30

### Designed Oligonucleotide for Experiment

### SEQ ID NO:31

### Designed Oligonucleotide for Experiment

### SEQ ID NO:32

### Designed Oligonucleotide for Experiment

### SEQ ID NO:33

### Designed Oligonucleotide for Experiment

### SEQ ID NO:34

### Designed Oligonucleotide for Experiment

### SEQ ID NO:35

### Designed Oligonucleotide for Experiment

### SEQ ID NO:36

### Designed Oligonucleotide for Experiment

### SEQ ID NO:37

### Designed Oligonucleotide for Experiment

### SEQ ID NO:38

### Designed Oligonucleotide for Experiment

### SEQ ID NO:39

### Designed Oligonucleotide for Experiment

### SEQ ID NO:40

### Designed Oligonucleotide for Experiment

### SEQ ID NO:41

### Designed Oligonucleotide for Experiment

### SEQ ID NO:42

### Designed Oligonucleotide Primer for PCR

### SEQ ID NO:43

### Designed Oligonucleotide Primer for PCR

### SEQ ID NO:44

### Designed Oligonucleotide Consist of Objective DNA Domain

### SEQ ID NO:45

### Designed Oligonucleotide Consist of Objective DNA Domain

### SEQ ID NO:46

### Designed Oligonucleotide Consist of Objective DNA Domain

### SEQ ID NO:47

### Designed Oligonucleotide for Experiment

### SEQ ID NO:48

### Designed Oligonucleotide for Experiment

### SEQ ID NO:49

### Designed Oligonucleotide for Experiment

### SEQ ID NO; 50

### Designed Oligonucleotide for Experiment

### SEQ ID NO:51

### Designed Oligonucleotide Primer for PCR

### SEQ ID NO:52

### Designed Oligonucleotide Primer for PCR

### SEQ ID NO:53

### Designed Oligonucleotide Consist of Objective DNA Domain

### SEQ ID NO:54

### Designed Oligonucleotide Consist of Objective DNA Domain

### SEQ ID NO:55

### Designed Oligonucleotide Consist of Objective DNA Domain

### SEQ ID NO:56

### Designed Oligonucleotide for Experiment

### SEQ ID NO:57

### Designed Oligonucleotide for Experiment

### SEQ ID NO:58

### Designed Oligonucleotide for Experiment

### SEQ ID NO:59

### Designed Oligonucleotide for Experiment

### SEQ ID NO:60

### Designed Oligonucleotide for Experiment

### SEQ ID NO:61

### Designed Oligonucleotide Consist of Objective DNA Domain

### SEQ ID NO:62

### Designed Oligonucleotide for Experiment

### SEQ ID NO:63

### Designed Oligonucleotide for Experiment

### SEQ ID NO:64

### Designed Oligonucleotide for Experiment

### SEQ ID NO:65

Designed Oligonucleotide for Experiment

1. A method of measuring the content of methylated DNA in a target DNA region in genomic DNA contained in a biological specimen, comprising:
(1) First step of selecting methylated single-stranded DNA, by First (A) step of separating methylated single-stranded DNA from a DNA sample derived from genomic DNA contained in a biological specimen, and First (B) step of causing binding between the single-stranded DNA separated in First (A) step and an immobilized methylated DNA antibody,
(2) Second step of mixing the single-stranded DNA selected in First step with a masking oligonucleotide comprising a nucleotide sequence complementary to a nucleotide sequence of a recognition site of a methylation sensitive restriction enzyme and digesting the selected single-stranded DNA with at least one kind of methylation sensitive restriction enzyme, and
(3) Third step comprising as pre steps of the following regular steps:
Step (Third (pre A) step) of separating single-stranded DNA (single-stranded DNA not containing an unmethylated CpG in a recognition site of a methylation sensitive restriction enzyme protected by the masking oligonucleotide) which is an undigested substance obtained in Second step from the immobilized methylated DNA antibody and the masking oligonucleotide into DNA in a single-stranded state (plus strand);
Step (Third (pre B) step) of extensionally forming double-stranded DNA from single-stranded DNA (plus strand) containing the target DNA region by extending DNA (plus strand) derived from a genome that is made into a single-stranded state in Third (pre A) step by one extension of an extension primer using the extension primer (forward primer) comprising a nucleotide sequence (minus strand) which is complementary to a partial nucleotide sequence (plus strand) of the nucleotide sequence comprised by the single-stranded DNA (plus strand), the partial nucleotide sequence (plus strand) located on further 3′-end side than 3′-end of the nucleotide sequence (plus strand) of the target DNA region, as the extension primer; and
Step (Third (pre C) step) of temporarily separating the double-stranded DNA extensionally formed in Third (pre B) step into single-stranded DNA (plus strand) containing the target DNA region and single-stranded DNA (minus strand) containing the target DNA region, and as regular steps:
(a) Third step-A of extensionally forming double-stranded DNA from single-stranded DNA containing the target DNA region by one extension of an extension primer by using the generated single-stranded DNA (plus strand) containing the target DNA region as a template, and the forward primer as an extension primer, and
(b) Third step-B of extensionally forming double-stranded DNA from single-stranded DNA containing the target DNA region by one extension of an extension primer by using the generated single-stranded DNA (minus strand) containing the target DNA region as a template, and an extension primer (reverse primer) comprising a nucleotide sequence (plus strand) which is complementary to a partial nucleotide sequence (minus strand) of the nucleotide sequence comprised by the single-stranded DNA (minus strand) containing the target DNA region, the partial nucleotide sequence (minus strand) located on further 3′-end side than 3′-end of the nucleotide sequence (minus strand) complementary to the nucleotide sequence (plus strand) of the target DNA region as the extension primer,
wherein by repeating the regular steps after temporarily separating the extensionally formed double-stranded DNA obtained in each of the regular steps into a single-stranded state, the methylated DNA in the target DNA region is amplified to a detectable level and a content of the amplified DNA is quantified.

2. The method according to claim 1, wherein the methylated DNA antibody in Third step is a methyl cytosine antibody.

3. The method according to claim 1, wherein the biological specimen is serum or plasma of a mammal.

4. The method according to claim 1, wherein the biological specimen is blood or a bodily fluid of a mammal.

5. The method according to claim 1, wherein the biological specimen is a cell lysate or a tissue lysate.

6. The method according to claim 1, wherein the DNA sample derived from genomic DNA contained in a biological specimen is a DNA sample preliminarily digested with a restriction enzyme whose recognition cleaving site excludes the target DNA region comprised by the genomic DNA.

7. The method according to claim 1, wherein the DNA sample derived from genomic DNA contained in a biological specimen is a DNA sample digested with at least one kind of methylation sensitive restriction enzyme.

8. The method according to claim 1, wherein the DNA sample derived from genomic DNA contained in a biological specimen is a preliminarily purified DNA sample.

9. The method according to claim 1, wherein the at least one kind of methylation sensitive restriction enzyme is a restriction enzyme having a recognition cleaving site in the target DNA region comprised by the genomic DNA contained in the biological specimen.

10. The method according to claim 1, wherein the at least one kind of methylation sensitive restriction enzyme is HpaII or HhaI which is a methylation sensitive restriction enzyme.

11. The method according to claim 1, wherein Third step is executed without conducting the digesting with the methylation sensitive restriction enzyme in Second step.

12. The method according to claim 1, wherein a counter oligonucleotide is added when methylated single-stranded DNA is separated in First (A) step.


SEQUENCE LISTING (65 sequences)

SEQ ID NO: 1 - DNA, 2661 bp, Homo sapiens
acagacatgtgccaccatgcccagctaattttttgtttgtttgtttgtttgtttgtatttttagtagagatggggttttgccatgttggccaggctggtctcgaactcctgacctcgaatgataatgatccgccgcttggcctccaaagtgctaggattacaggtgtgagccactgcgccaggcctgggcactttctttagtagtttgaggagcaacatttttgacagtgtccttctgctcaagattcaagatcccagataaaattaaaccatctagagagatggcttgattggccaaacctggatctcatgaccacttcttgaagtgggtaagtctcataaatgctcagtccttccactatgcaactgagtggggtgggtgggaagcccctcaaaggaaaatccggttgttcttactagaaagaaaaggaaaatggatgtgaggcagtcaaaatcagcagaggtccaccacaccaccaaaatgtggtgattaaatatggagagacagagactaacagaggtatgtgaatattgaagtatgtctggacaatagcccaatgatgagaccaataaaatggttaccaaaatctggttttgagtagtagtgttaaatcagaccatttagtaaccattttttgttgcaaagtttctagcactgcccaaaccctgagtggtatatgaataactcgtccattatgtatctctttccagtcagcataatttatcccccacctatattcttttctgaccactcctacttccttctctttaccaaaatctaaactctaaggctgtttcttcagcaacttctttgtttagattggaagataaattaaacagcatgcgatgttttactgactttcagtatttaacagaggtgatttaatttttttttaaatccaaagtcaaacttctttataagatgaaggagaaaaatgtcttataaaatgcatatgtgaagatgccttctgagtgctttctcatgcagacttgttctagtctttaatgaatcttccttgtagacactgtggagatgaaagatggttctccacttctactcaaagtacaaatcaggccggcattttgaaaaagagacaggtttattcatagctgcagcgttagctggctttgttccctgtacaatttcacttttggttattaaaatattcactgtaggaaataaatttgtaacccatttctcatattacctacacacagaaaaacaaaatttgatatcctggggtttatttgctgagggcgcttcccataaaagcgagagagtgtgcgttgggaaatgtgtctggttaactcttttatggataaactttagtcacaatcctcccccgcccccctctcacccccagcaccctcccaacctcccgacttcccgcctctcaagggctggtgacctaatagcatttttcttcgtgcatattttggcgtcgccccatggcctggctgccttcgcctgtctgagttttttgaaattcctgcatgttcgccccagattaagccagtgtgtctcaggatgtgtgttccgttttgttctttccccttaacgctccctgtgcaacgtgtctggggggaggagggcagggacgggagagagggaggggcagaggcgaggagctgtccgccttgcacgtttccaatcgcattacgtgaacaaatagctgaggggcggccgggccagaacggcttgtgtaactttgcaaacgtgccagaaagtttaaatctctcctccttccttcactccagacactgcccgctctccgggactgccgcgcggctccccgttgccttccaggactgagaaaggggaaagggaagggtgccacgtccgagcagccgccttgactggggaagggtctgaatcccacccttggcattgcttggtggagactgagatacccgtgctccgctcgcctccttggttgaagatttctccttccctcacgtgatttgagccccgtttttattttctgtgagccacgtcctcctcgagcggggtcaatctggcaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaacctggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacg

SEQ ID NO: 2 - DNA, 1953 bp, Homo sapiens
tataaattccacgcaggcattgaattgaatttgttcttaaccaaatgcgttttatctatacctggcaggaatctagaagtgaaattacaagatttatttcattttaattctattatgaagcatttaatcacaaataccctgaaaatgaaaagataatttatcattttaccttgactgagcaactctcctcacttcacattcatgaatccataacgcagagaggagactggatgattaagtgtttgattagagaaaacagattaacctagcaaacataataaatttggctcataagcaggatggctttataaatgctcacaatacctctcctgtataaaatcatgaaccacttcctacagtgatgactccatcgaaatagttgagaaacataaagcaaatgcatgtttatggctttctctttgagacattaaaagggtattgaaaggcatatctgattcagcttataactctggatatatattaaggaacatgtaagaaaatattaatgcataaaaaaagctacaacttctcaagtgttctagtttccactttgtcaataattacgttttcaatgtccttctgtggactgtttccaaaggtgccaatccagacccaaagtttcagatcactcagattcacccttaaccttcataacacaacccaatagctttacgaaaaaagttgcatatttaggtagttgttatcccattatgacaaaatacataaaattagcgagatattttttagccttcaaataagtgggaaaaaatccttttagctgagattccatttacatcagaataaaaatctaagttatgactaggttgaagcaacgtcctgtgcagcgctccataaagttcacttagtcttcaagggttccttacttagctaggttagtattcctggcctctttttttagcagtgagaaaaaggatactctccctgccccagctttatttttaaactcacagccatatcctggaggtctctgctggctatttggcgcgtgggggcggaggggggccgggggaggggggcggggcggggtctggaggtctgtgctggctatctggcgtgtgtgtgtgtgtgtgtgtgtgtgtgtgtggttggaggtctctgctggctatctggcgtgtgtgtgtgtgtggtgtggtgtgtgtaagcagtgaggttgttttagggccagtccttcctccgccactttgctgactcaaagacccagaggctttcttggggtgcaggtaccatgattccttgggccctaagggaatttttgttaggctagaagagtgggtgtactcatgatgggtgtacccgaacattcctgggctcaacaaaaccgattatctttataaccgcggcgcctagcacagcgcctggtgccctaaacgttggctgcgggaacgtccgagacgcgggtgcggagccgggggcggaataactggttgcgcggcgctttgaccgtaggcgctggagcgcgtgcgttgcgtgcgcgcgcggaggcggctgcgtcggggcgcgagaaggtgcagttccccggcgggcgggcgggcgggcgggcgaagctgggctcggggccaagcgaggtctagccggagcgactgtgccccgcctcctgggcggagcgggcggctccccatggtcagagcctcgtgccggctcggcagcgcccggacgccgagcccagcgcgtcggccccccggcgtgcgggcgtctcagagccgcggaggggccgccgggaccgtttcagcgtggcggcgctggtgctggcgttggccctggaggacggccccgagtgatggctggcgcctgcctcccgggtgtctcccgggtacagatggagtcgtcccgcggccgccggcggcaaggtcggcagctgcgaggccaagagagaccccaggacacacacagctgcctcccggtgcgagaagaagaccccggcttgagagtgag

SEQ ID NO: 3 - DNA, 889 bp, Homo sapiens
cggccccatggctccgtgtcgtgtccaagggatgggctggcacctcttggaccaggcttaccaccagggcccttctctgaagccccagtctgaccggcctgctgctgggaatccccctctgcccccacactaacctctgctggggctgagccagggcgcgtcggacagtcagggcgacccagccagggcgaccgttggccccgctcctatggggcagcagggaccgacgtcagcagggtggggcgggcacccgagtggtatgccccgccctgccccgcctgcccgccctggtggccgtctgggggcgacaagtcctgagagaaccagacggaagcgcgctgggactgacacgtggacttgggcggtgctgcccgggtgggtcagcctgggctgggaggcagccccgggacacagctgtgcccacgccgtctgagcaccccaagcccgatgcagccacccccagacgaggcccgcagggacatggccggggacacccagtggtccaggtgtggcgggggtgaggggagggggggtgggagcggtggagatggggccgtggggagggagctgagatactgccacgtgggacgatgctaggtggggagggctgagctgggcgggctcctctggctgtggggccccctgtgttccttgtgggaggtggaaggaagtgagtgccctgtccttcctccctgccatgagattccaggaccggacctggcaagtgccctatcccagccagtgttcctggggctcttccaggcagggctatgttccccaggccaggggcattgtcctggacagtcaggaggcatacccctcgccaggtggaaccaccctgtgtatgcatgaccctgacaagcaggcgccaggacagtcaggaggccag

SEQ ID NO: 4 - DNA, 863 bp, Homo sapiens
gttgttgggtgtgaatggagaactgtgggccctccccgacaccttccagcgggacggcaacgggggcccagggggtgggcgccatcaaccccgtcccaccgccaggacggcgcgggggagggccggcgggggcggggcgtcctgtaaggcgcggcccccacccgcgggcggggcggcattcctgggaggccggcgctctgacgtggacccgggggccgcgggcacggcgggggggcggcggtccgggggcttcttaaaccccccgccccggcccagcccgcacttcccgagcaccgctccgaccctggagggagagagagccagagagcggccgagcgcctaggaggcccgccgagcctcgccgagccccgccagccccggcgcgagagaagttggagaggagagcagcgcagcgcagcgagtcccgtggtcgcgccccaacagcgcccgacagcccccgatagcccaaaccgcggccctagccccggccgcacccccagcccgcgccagcatgatgaacaacagcggctactcagacgccggcctcggcctgggcgatgagacagacgagatgccgtccacggagaaggacctggcggaggacgcgccgtggaagaagatccagcagaacacattcacgcgctggtgcaatgagcacctcaagtgcgtgggcaagcgcctgaccgacctgcagcgcgacctcagcgacgggctccggctcatcgcgctgctcgaggtgctcagccagaagcgcatgtaccgcaagttccatccgcgccccaacttccgccaaatgaagctggagaacgtgtccgtggccctcgagttcctcgagcgcgagcacatcaagctcgtgtccatag

SEQ ID NO: 5 - DNA, 2198 bp, Homo sapiens
aagagaggcacactccctctaccacaccgagggagggggcgttgagctgagaaaggttgagagaatgagggacccaggtaggtggacatcggccaagaaaggaaccacagcgggaggtaagaccgagagtccccagcttgaagcgtcaccactccgggattcccagattccaacgcgagcctggggaaagcccacagtggagagagtccggctggcagggaatggccctacccccggggtgaaatctcggagggtcgtgcagccgagtcgcgcctctgcgctgatgcgtgagagatgccggacgtcgcgtttgcctgtgcgagcctcgcggatgctgtgcagtcttggtcccctctgcgtgtgtctaacgccgaatgctggtgtctcgaggtgtgagcttcggggccggtgtctttaaagaaccaaagattcttaaggagtgatgatctgggtagagcggcccgacgtagccgcgctcccaggtctcggtgcgagtcctgcggacagaccagaggagacctgctggccagatgccccgggcccaaggcggacgccagactgtctctgcgccagccgggctggccttcggaatggatcaggcacccgggaggccggagtggatctcagaccctcaagccgggaacaaacccgtcgatgcccgtgggcctggagtccgcctcctccttcccgccccacccctacccctgcctccgaaaggcttcttcgctggtcagtagctgcgtgcccgtctgcctgaggctgggtcagaattggcgggctggtaacgaccccgtgcacaagcggctcccagtctctccagaaagggccgatgactaaggggtgggggtgggggcggagggctggaaggtgttagggaagaacgttagcggcctatcctgtcttcagcagcgccctctcatcttctagctctgacgccgagcagagcagttggagctcgggactgggaactgctggaattcctatttagacttctagacagtctagaaacaagaacctttctttccctgggcctcagtttccttgtctgtaaaatcaaaaggcgggctctaggtgtaggccttcttttcgcttggtgattctggattcctttccttggatccgtggggagggggtggcagcaacagtccagggcgttggccgtcctgtgcctcaagtacgtagtccccgtgcccgccccctcaacacccccagcagcccgcccccctaagcccgcagagcagggagctgagtgggaggggcagaggcggggccggttcccagtccctgctggcggactagagtggcgcgggctgagcgtaaaacctgggatagccactcccccttttccttatccccgcccccctgccattggctcccgggagaggttgacatcaaagccgcggtcttatataagccagatccgcaggggagtccgcagaagggttaaacaggtctttgggcttcggcgacctcgcccgcggcagaaaccggtaagaagacagtgggctgcgcgtctcattttcagccttgcccggactctcccaaagccggcgcccagtagtggctccagagcccacaggtggcccccggcagtctctggggcgcatggagcggcgttaatagggctggcggcgcaggccagtagccgctccaacatgaacctcgtgggcagctacgcacaccatcaccaccatcaccacccgcaccctgcgcaccccatgctccacgaacccttcctcttcggtccggcctcgcgctgtcatcaggaaaggccctacttccagagctggctgctgagcccggctgacgctgccccggacttccctgcgggcgggccgccgcccgcggccgctgcagccgccaccgcctatggtcctgacgccaggcctgggcagagccccgggcggctggaggcgcttggcggccgtcttggccggcggaaaggctcaggacccaagaaggagcggagacgcactgagagcattaacagcgcattcgcggagttgcgcgagtgcatccccaacgtgccggccgacaccaagctctccaagatcaagactctgcgcctagccaccagctacatcgcctacctgatggacgtgctggccaaggatgcacagtctggcgatcccgaggccttcaaggctgaactcaagaaggcggatggcggccgtgagagcaagcggaaaagggagctg

SEQ ID NO: 6 - DNA, 1945 bp, Homo sapiens
ctggatgacagagtgagactccgtctcaaaaaaaaagctccatttgggaggccgaggagggtggattacctgaggtcaggagtttgagaccagcctggcccacatagggaaaccccatctctactaaaaatacaaaaattagtcaggtgtggtggctgacacctataatcccagctacttgggaagctgaggcagggagaatcacttgaaccggggaggtggaggttgcagtgagctgagatcatgccactgcactccagcctgggcgacagggtgagattctgtctcaaacaaacaaatttaaaagctccgaatcctccaaaaataccaagattttcctgtcggtaactagagatgggtactgatgattatttttaataggtgattttcaaagatgtgaacgttatccatggagatttaagtctccaaaaggaaaaaaaatgcatacctttatactaaaacttcatcaccagtcaaatttggatcatcactaaattggcttctacacctctctcctaatataaggtacttgtgtaagtttgcagttgtgagacacttatttcctcatttttaatgtcttctcagtagggccactgatatagtcactatttgactgaccagaatggttggcactggtgattggctcataaagtgccctcgatttagggggctcaattatcaaaggtttaaatcctagcccaaaccattgctgtgatgggggttaatcaatgaaccactcagcttcacttgcaaaagcgggatcacaatagccgctttcgtcatgacccagcctaggtgagatttagtacttaagtacactgccaggcacacaaggttaatttaacaatttaacacatttgtttcctcatccatttctccaaaccttccaactaatcctaacgttcgttcggccaaatgggccaggaattcacttaaacaaaaacaaaaaacaaaacaaacaaaaaaacactccctggggcttggggaaggaggcaccgccgcccatgtcgcagtctgggggtggctcagtcctcagcacccagatctacggccataatgctcttcgaggccaaggagcccggatgcggggcgttgccgaaggcgtcttgctcaggctgcgggaaaggagaggggtgggagcggggtgggggcatcgcgacccagggcaaggcggcgagtcgccgtcttcgagtcccacctgtccgaagcggggtgagaaaaggcaaaacatggcaaagccatgcacctcccagggtgggcaactcacggccggtgaacgccggacccttagcagtttccagacctttggaaccggaagcggagcctgagagcgcgcccgagagggcgtgaacgggaccgctttcccggaagtgcttgcggcctctgcccagcgagctgccccggggtctctctggtttcctaatcagggcaacgccgcgggagagaacctttaccttggctgcactaagttctcggtgccactccctggcagggcgggaccttgtttaggccctgtgatcgcgcggttcgtagtagcgcaaggcgcagagtggaccttgacccgcctagggcgggaagagtttggcccgccgggtcccaaagggcagaatggacgggctcctaaatcccagggaatcctctaaattcattgcagaaaacagtcgggatgtgtttattgacagcggaggcgtacggagggtggcagagctgctgctggccaaggcggcggggccagagctgcgcgtggaggggtggaaagcccttcatgagctgaaccccagggcggccgacgaggccgcggtcaactgggtgttcgtgacagacacgctcaacttctccttttggtcggagcaggacgagcacaagtgtgtggtgaggtacagagggaaaacatacagtgggtactggtccctgtgcgccgccgtcaacagagccctcgacgaag

SEQ ID NO: 7 - DNA, 2379 bp, Homo sapiens
aagcttgtggtttacttggacctctgcctcatctttcttcttttgcgcttcagcctgcgcattcgcttcctccactaggctctcatggtgcagaggtttccaagaagatggtgtgaaggccgagatcatttggttatattataaaatagaatgcaaattcacacaagtttttgttttttatttatttatttttttagagatgaggtcttgctatgttgtttagtctggtctcgaactcctggcctcgtgatcctcccaccttgacctcccaaagtgctgggattacaggcctgaggcctgagccactacacccaactgaattcacatttttttttttcttttctgagacggagtctcactctgtcacccagtatggagtgcagtggcgcgactgcggctcactgcaagctccgtctctcgggttcaagtgattctcatgcctcagccccccaagtagctggaattacaggggtgcactaccacacctggctaatttttctgttttagtagagatggggtttcaccatgttgcctggtctcaaactcctgactttaagtgatccacacacctcagcctcccaaagtgctgggattacaggtgtgagcctccacacccggccgaattcacatgaattttaaagtgatgtcttcaaagtggtttcactgtggggatgggcagctttttgttatacatctagaacgttcctcttctgtttctatgaatactcggttggaaagggctgaaaaacggtcttaagagattatctgattcgtttcccagttttattactcacatatcagctgtaatttgagcacgttttctgattgagacaagactcagatggtattaaacattactacaacacatccgggcacggtggctcacgcctgtaatcccagcactttgggaggccgaggcgggcggatcacgaggtcaggagatcgagaccatcctggctaacacggtgaagccctgtctctactaaaaatacaaaaaattaggcgggcatggtggcgggcgcctgtagtcccagctactcgggaggctgaggcaggagaatggcgtgaacccgggaggcggagcttgcagtgagccgagatcgcgccactgcactccagcctgggcgacagagcaagactccatctcaaaaaaaaaaaaaaaaaaaaaaaactacaacactataaattcatatctattataatagtactttgtgcagggccctaccctaagtccttaaccgaacccggaagcgagaagatgacttttgtttgtttttagagatgggcgcctggctctgtcgccagcctggagtgtgggggcgcgatctcgactcacagcagcctccacctcccgagttcaggcgatcttcctgcctcagcccctcgaggagctgggaccaccggcgcgctccatcgcgcccggctaggagctgactttgaatccgggctctgcgcctggccttctgcatctctataagggaagacatctgtgacctcggggcaaaggtcaaattagatcctgggtaggatcctgttcccgctgcccctcgggctggcactgccaggagtactcagagctcaaagctgggatctgcagtcccttacccactcagtgcacgccgcctaaggctttgcgcttcacctttactcacctcgaagccctggacatccgcatctgccctaagacttctcacctcagtagcagaaggaagtcgcgtcagctggccacagcctctctcctaggagaccgtccgggaaaagcgagtcagggtagaccctgaggcccctcagctccggctattttcagatctgtcgctccttcaccctcagcctttcaaacaggccactccaaaaaaaagcccaatcacagccttccttcttctcctggccttccggcactgtccaatcaacgtacgccatctatcggttagtggtgttgcggggccacccttcccgctggtttccctcgtggtgtgtaaaggcagagaggaaaggcgaggggtgttgacgccaggaaggttccatcttggttaagggcaggagtcccttacggacttgtctgaggaaagacaggaaagcgccagcatctccaccttccccggaagcctccctttgccaggcagaaagggtttcccatggggccgcccctggcgccgcgcccggcccacgtacccggggaggccgggccccggaggacgagggaaagcaggccgggcgccgtgagcttcgcggacgtggccgtgtacttctctcccgaggagtgggaatgcctgcggccagcgcagagggccctgtaccgggacgtgatgcgggagaccttcggccacctgggcgcgctgggtgaggccgggccctccggccgggacccccagtccgtcg

SEQ ID NO: 8 - DNA, 933 bp, Homo sapiens
gagacgtactctggctctgtcgcccaggctggagcgcaatggcgccatctcggcgcactgcaacctccacctcccgggttcaagcgattctactgcctcagcctcccgagtagctgggactacaggcgcgcactaccaagcccggctaatttcttttgtatttttagtagagactgggtttcacgatgttggccgggctggtctggaagtcttgacctcaagcgtgcgccctctccgccactgggtaaggcggggggcggaatagggggcttgcaatttcacactagaggcgggcgccgtgggggaaagaagagtcacgtctcccacggttcgtagaggaaggcctgcctgagcctggagcgggggcgggagagccacagtttggcatccccagggcatcccccagcccgcagactaccaggcctccagaggacaggaccccacccccggccacaggccctgcccccagcactccccgcaccccgcctccaagactcctccgcccactccgcacccaacttataaaaaccgtcctcgggcgcggcggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcggctcggccaggggctgcgcgaacacgcggagcgcacccgcagccagctgagcgcgctggagcggcgcc

SEQ ID NO: 9 - DNA, 6096 bp, Homo sapiens
atctgcacctcctcatatagggttgatccaagtttcacagacatcactgagttcttagtggactcagctattggggctgttctcacactttttttttctttgcaagaatcagcaatgggtgcaagtggacctgtgtaggacgtccagtgaaacattgtgttggtgaatcagctagaatccatccaagaactcagccagcctggtgtggggtgagatctgatccttgaatgtccctcagtggcttttagggctggcaggttcagaagggccctctcatcacccccccagggcctcattccttgtttaacactttgctatcacagtcttgaatccttgtaattgaacaatggaccccacattttcactttgcactggtttctgattctgtaaccgatcctgtccccctctcttgtctcattcactctgggaattgtccccacattctgagacctttcagcagtgccccaacgaggttcctgcccttatctgaagctccaccctcacccccatggcggcaccgcaggcagccctgcttttgcgtcccgcgtaggcaggctgtgcaccggagtcacgaccccctgattcagcctaggcagccacagcttgactgctcccgccggacaagccctactgtgctatctgccgctcttcccttcctcttcccagggggtccgcgtcaggggaggcgcagctgtgtgcattccgggagcttcagacccccgtgtccagcagctccttcgtttcctgggtgctggggcggccttcccagcgaagagctcaactcagcgggacgtttggaggctctctgccccaaggcgctggggagtgtgcggcgggacagtcgtgcttgcctttttcactttcagagtgtccacgccccacccgtttggtcactgcaggtcagtccagtccagcccggcccaccccaccggtgcgtgtctgtcgcacgtggcagacgccatactctctgttcttgtttaaagcccaggatctactgggccctggaggcaagaggtgaacgcagcggaatccacgctgagctgcccgggaacggagcttccaaccccagaaggaggactctgtgctcctacaccttaaccctttttagcccgaaacttctccaacttccttggctttgtttagagctcgacagcgccgccccctggcgctcgttgtgaggacagtagaggagagaggcaagggtgtttttaaacagtttgcctctcaccattatgggggcgacccgagggggagacccactcttccgcattcccggtaagtgaaccaccggaagaggtcgaaagtgacggattcccatgtcctcctccagccccccccccaccctgcccatccacaggacggtggctcttcagtgccctttgccgagcaagtggcgtttctatgcacgtgggtatcaattcggactctggacgaaatggaaacctccttagccgacccgggtgggatcagctgggatcctgcgcgctcccctggggggttgccagccactctgttggggtgcaagaagcaccatccttcggaagctgggccgaaactggccaggctgactcgctcccacgcgcccgcccctacccggcgccgcagcaattcacctgccaccgcctctgagccgggtccggacttcggcgccctgacagtgtccccgcgacttccccacccgatgagatggggtctggcgttggccagtgcgtgtccagggactcgcgggtccctggccagccatggggcagagggcgctggtgttaggccagtcttccccaccctgccccgtcaccccagccacacccactgtcctgtgaggccaagcgcgctccgctggtttcctgagccaggcaccttggccgcggacaggatccagctgtctctccttgcgatcctgtcttcggggaagtccacgtcctaggcaggtcctcccaaagtgcccttggtgccgatcacccctcccagcgtcttgcaggtcctgtgcaccacctcccccactccccattcaaagccctcttctctgaagtctccggttcccagagctcttgcaatccaggctttccttggaagtggctgtaacatgtatgaaaagaaagaaaggaggaccaagagatgaaagagggctgcacgcgtgggggcccgagtggtgggcggggacagtcgtcttgttacaggggtgctggccttccctggcgcctgcccctgtcggccccgcccgagaacctccctgcgccagggcagggtttactcatcccggcgaggtgatcccatgcgcgagggcgggcgcaagggcggccagagaacccagcaatccgagtatgcggcatcagcccttcccaccaggcacttccttccttttcccgaacgtccagggagggagggccgggcacttataaactcgagccctggccgatccgcatgtcagaggctgcctcgcaggggctgcgcgcagcggcaagaagtgtctgggctgggacggacaggagaggctgtcgccatcggcgtcctgtgcccctctgctccggcacggccctgtcgcagtgcccgcgctttccccggcgcctgcacgcggcgcgcctgggtaacatgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactctgagcggcctccgtccaggagcctggctccgtccaggagcctgtgcctcctcacccccagctttgctaccaaagcaccttagctggcattacagctggagaagaccctccccgcaccccccaagctgttttcttctattccatggctaactggcgagggggtgattagagggaggagaatgagcctcggcctcttccgtgacgtcactggaccactgggcaatgatggcaattttgtaacgaagacacagactgcgatttgtcccaggtcctcactaccgggcgcaggagggtgagcgttattggtcggcagccttctgggcagaccttgacctcgtgggctagggatgactaaaatatttattttttttaagtatttaggtttttgtttgtttcctttgttcttacctgtatgtctccagtatccactttgcacagctctccggtctctctctctctacaaactcccacttgtcatgtgacaggtaaactatcttggtgaatttttttttcctagccctctcacatttatgaagcaagccccacttattccccattcttcctagttttctcctcccaggaactgggccaactcacctgagtcaccctacctgtgcctgaccctacttcttttgctcttagctgtctgctcagacagaacccctacatgaaacagaaacaaaaacactaaaaataaaaatggccatttgctttttcaccagatttgctaatttatcctgaaatttcagattcccagagcaaaataattttaaacaaaggttgagatgtaaaaggtattaaattgatgttgctggactgtcatagaaattacacccaaagaggtatttatctttacttttaaacagtgagcctgaattttgttgctgttttgatttgtactgaaaaatggtaattgttgctaatcttcttatgcaatttccttttttgttattattacttatttttgacagtgttgaaaatgttcagaaggttgctctagattgagagaagagacaaacacctcccaggagacagttcaagaaagcttcaaactgcatgattcatgccaattagcaattgactgtcactgttccttgtcactggtagaccaaaataaaaccagctctactggtcttgtggaattgggagcttgggaatggatcctggaggatgcccaattagggcctagccttaatcaggtcctcagagaatttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatccttgcttatgacatcacttgtacaaaataaacaaataacaatgtg

SEQ ID NO: 10 - DNA, 2500 bp, Homo sapiens
acccacttctgtgtgtggatagtatcctgcaggagagatgttgtctgcagtgtgagctgggcccaccggagtgtgtgaataggatcctgcaggagaaatggaatccggagtgtgagctgcatccgctgtagagggtggataaaatcctgcaggaaagatggcatctggaatgtcagcgggagccaccgacctctgaggatgcaccccgcaggtgtgatgcggggccagttccaaggctgggttaggttttaccctggcttctgtgttgtactctcattctcttcctctttcttctaatacctgctctgggaggcatcaggccatgtccagtgtgcaggccatggagacccacacggcaaggaactggaaccccctgccagcagcctcgggggtccagtccttagatggtgccctgtggtcagcaatgcacctgtgacctccgggctatgtctcgtggtagttgcttttgtgttttaacatagcaacaggaaactagcctattacccaccaatcccattccaggctgctttcaaacgcagctcaggctagaacaccagcacggggacacagctgagacttggggtttgcgacgggaacacgcccatgctgtgcctctgaatctggcaccgtcaccctgtggcctgggttcagcaacttggcctcaccttccttgtctgtgaaattcagactgggtccttgtgagatgattggagagaatgtatgaactatgtgagaacgccacctttgtgcgtatctcacgcagtgtcttccctcctttccaaagtcttctgctgtctctagacacacccgacgtggggggggggggttccctgggtctcctcctaggtctgtcccaggagggcacgcactgaaggccgcgagaatcccgggggctgcattgcgccgcgccaaggactccacacaggacctttcattttcccaactgtgctgagccaggcggccggcagagagcaggtggctgacaggccccggggagccggaccgcctgggtctaatcttcccgcagactcccttgctgtgcgctttggggcttgggcctcagtttcctcaaaaggaatgaggggcttttttggaacgttaaataatttcctacgtggttgcgggtagggagaaggagaaagagaggagcgcgcctgcgcgcctggaatcgtgcccggatcagagcaagcgctctaaaagtgttacaaacattaaggcgccaactaaaaaacccgtagtgagcgcaggcagaaaccacgggtaagagaagtggagaagcttcgcgtaggccccagggtcccgagccccgagtctcgagcgcagaatcaggggtgccaatgctctcctccgcgcccccgagcgctcgccttggccatgcgggccgccccaccgggatgagggcgctcaggccggacgctggggccccgggttctcgccccgccccgccctcggggattcagaggggccgggaggagcctcgcgcatgtgcacagctggcgccccccgccccccgcgcacagctgggacgtgggccgcggccgggcgggcgcagtcgggagccggccgtggtggctccgtgcgtccgagcgtccgtccgcgccgtcggccatggccaagcgctccaggggccccgggcgccgctgcctgttggcgctcgtgctgttctgcgcctgggggacgctggccgtggtggcccagaagccgggcgcagggtgtccgagccgctgcctgtgcttccgcaccaccgtgcgctgcatgcatctgctgctggaggccgtgcccgccgtggcgccgcagacctccatcctgtgagtgccgcgggggacgccgggggcgcggggtccggggcttcgtggagatccgggagcgcaggggtgatcggaggtggggggcgcggagggtggagggggcatcgggcgcgcggggggcctggggacttgggacgcagaagggaacctccgaagggggacgtggggggacctgggcgcggggacccgctgggcctttgttcgccctgcgggagacgccgaggggcggaacagagcgctgtgcgcgcggccttcgtagccgcctttgttcggaactcggaatccccgcaggactgggaagttgttggagcctccggggctccccccgctcgcctcccgccgccccctctcatgctccgccggcctcccgcttccccctggttcgcggcccctcctccgctcacctttcccccgctcaggacccctcggtccccctccgctccccgagcgcggcgcagccccctccgtcctcccagccccctccgccccgttcctcgtcctgttcgctcccctcctccgctcctcttcctcctccccttcctcctcctcctccccttcctcctcctcctccccttcctcctcctcctcccttcccctcctcctcccccccttccttctcctcccccagcctccgccctctccccctcccccgccccttggagcgcagtgcccaccccatccccccgcgccgg

SEQ ID NO: 11 - DNA, 2200 bp, Homo sapiens
cctcgccccctccagccggccccccgggcccctcctctcggcgcccggaccttggccctccctctcctttcccacttctctctttgccctaacttcgcccccatcccccgctcatttcctctcgcacccgggctcgccaatccctctttccaagtccctcttccagcccggccttcctctcgggttcgccccccttctccccaatctccgtcctcttccctcccttcgccctcccccccttccttcctcttcccctcacccaaccctggttcccctcgttcctcagtcccgatctctcccttactctgtccccgcccactctgcgccggcctctcagtccgggttgagccccacgtgtggacggccgcgcccccactgacagccgccgcccgccggcccgccccgcgccccgccgggcctctaaaacccccgcgccgcgccctccaccgccgcatcttctccagcgcccagcctcccgccctctctcttgctggccgcacgccccggccccgcgcacctccgcccggctccgcagccgctacccgcgcttcgttgccctgtgggactccgagcgagcccggagggaaccctcctcttcttctgggggcgacttttgtttgcttgcctgtttctttctggtgacttttgcagctttccaatatccgtcttcggagcgcacgggaatccgccgagctctgcgtgcaggcccttttttcttttgaggttcacattttttgaaattttacgccagggcttttgtaatttcctcccccgcccgctgacggtcctggagtcgctcggggctttaggccggttatgcaacgtgtaccgctcggggctgccggctgcacctccgccgcgcctcgccgctcactgcgctagacccggcgccccgcgtctcgcttcgcgggcagtcagggggccggcgctctgtcgaggtctccagctagagcagggagcccgagcccgagggagtccccggagccgacgaagggcttattagaccctgactcttttctgaggcgcgcagattttgtctttgatcactccctctccgcgggtctacggccgcgcgctttcggcgccggcgatggggagaagacggaggctgtgtctccagctctacttcctgtggctgggctgtgtggtgctctgggcgcagggcacggccggccagcctcagcctcctccgcccaagccgccccggccccagccgccgccgcaacaggttcggtccgctacagcaggctctgaaggcgggtttctagcgcccgagtatcgcgaggagggtgccgcagtggccagccgcgtccgccggcgaggacagcaggacgtgctccgagggtaagtgggcaagcggctccgcacctagggctccggcttgggggaggggggaatcctcagtttggcggctttctggcccactccgtcccagaccctttagctggagcctagagctgcagccccctttgccagaatatccaaagacccccaggagcgcgtcccccttttccttcccaaccccgcagctcagcgggcggaaagccctctctccgggggttgggcggcgggtggttagggggtccaggggtgccgatcgcagagcgtgtgcagagctcgcgctgcgggaacaggttctgaatgtccggcggcaggcgggcctgggtccgcctgctgcaggggccagagaagcctgcttgctccccacgtcggggccgccgctcgtgagccttttgtttgaggacgtgtgcagggttcacagctcaccttctcatcgtcaacccgagcgctccaccttgcgacgcgctttccttgacacgtcggggccaaagtaacagttgaccaaggaggaatggatttgggaaggagggcaaggattctttggaacggaatggtccctttgttctctgcatctggaagctagaatagtagcaaattatatgtttccatgcctcttttcgccctttaaaaaggcaggcaagggacgacagatgaaaggcagtgtttagacatttctgaccctcctgcattccagcatctagctcttttgcttccacgtctgcctcccgatctccaataatttgaagtgtaattttgatttgtttgttgtcctgaaatctactcgctcggggcattgcttacgaagaccgtttatatgttgctgcatccctctacctatctgttacgtgaccgcgcttgt

SEQ ID NO: 12 - DNA, 2000 bp, Homo sapiens
ttggaagaaaaggatctccgaggaaggggctgagagaagggcagggtgaactggactaaaggccagagtaggaaggagaagaggggccaaaaaagaaggggatgaaattaagcacagaagatgggtaaagaaaaaagtatcagggaaagggcaaaataagagaaagccttgaggataagagggtagaaggctaaagaacaaggggaccactgggtcggggaagcgctgcctgaacggcgggacagtgacaaagaaagggcgctggcgatattcgcaccaagggtgcgaaacgcaatcgggaggtgagaaatggaaagaaggcgaatgcccggctacaagtagcctgggactgaaaggggacctgggggaggggctgggcccagggcagaaaagtccaggttcccatgcggcctgggcccacgtggagcgggcgctgaatcaccgttcagccgcccccctcccctcctccccgaccggtgcccgcagtccccgcctcctcggccgccgcctccacggggcggggccctggcccgggaccagcgccgcggctataaatgggctgcggcgaggccggcagaacgctgtgacagccacacgccccaaggcctccaagatgagctacacgttggactcgctgggcaacccgtccgcctaccggcgggtaaccgagacccgctcgagcttcagccgcgtcagcggctccccgtccagtggcttccgctcgcagtcgtggtcccgcggctcgcccagcaccgtgtcctcctcctataagcgcagcatgctcgccccgcgcctcgcttacagctcggccatgctcagctccgccgagagcagccttgacttcagccagtcctcgtccctgctcaacggcggctccggacccggcggcgactacaagctgtcccgctccaacgagaaggagcagctgcaggggctgaacgaccgctttgccggctacatagagaaggtgcactacctggagcagcagaataaggagattgaggcggagatccaggcgctgcggcagaagcaggcctcgcacgcccagctgggcgacgcgtacgaccaggagatccgcgagctgcgcgccaccctggagatggtgaaccacgagaaggctcaggtgcagctggactcggaccacctggaggaagacatccaccggctcaaggagcgctttgaggaggaggcgcggttgcgcgacgacactgaggcggccatccgcgcgctgcgcaaagacatcgaggaggcgtcgctggtcaaggtggagctggacaagaaggtgcagtcgctgcaggatgaggtggccttcctgcggagcaaccacgaggaggaggtggccgaccttctggcccagatccaggcatcgcacatcacggtggagcgcaaagactacctgaagacagacatctcgacggcgctgaaggaaatccgctcccagctcgaaagccactcagaccagaatatgcaccaggccgaagagtggttcaaatgccgctacgccaagctcaccgaggcggccgagcagaacaaggaggccatccgctccgccaaggaagagatcgccgagtaccggcgccagctgcagtccaagagcatcgagctagagtcggtgcgcggcaccaaggagtccctggagcggcagctcagcgacatcgaggagcgccacaaccacgacctcagcagctaccaggtaggaaccgcggctgcgcggccagcctgcgccagcgccagcgccgcgcgcccccgacacttgggctcgtgcccaggcgccctctccgccgcgctccctggtggccgctcgctagagcacgcgcgccgcagacctagggtatttgcggatcagcgtcctcgcccatctcatcctccacactccgcccccacccacctgccccagctgctaagggtcttgacctttttcagaaacgtgcatcttttccagttctaattttgcacgcttgcacgtttaaagcaggagggatgaattcggtagtggataa

SEQ ID NO: 13 - DNA, 2300 bp, Homo sapiens
tcagattgtcattgggagggtgaataaatgaatgcttgcattatgagagtttgggggcagaaatatgccacagactcttatctgaagccatcagatttagtggctgcgaacccaccgaagtcagggatttacattttttacagcaacgagagaaaacttcccctttcctctgcagaagtcaggactggatctcaaaaatagaaatgtgtcctcctaaatgtgtgcccatccccgtggttgacaaacaacggatttcccaagatagctgccacacacttggtttctaatctctgtattgcttccccgccagaatgtcgaagtccttcccgaatatgcccagtcatactttctgaacttttgagcaaacaccgtccggcttcttgtgctttcctcaaagaccccaggcaccggcagggaggacacaggccggggcagagcgcccctgcgcgggggattcctgccactccgcgccagcctgcggcgcaaacgctcttctcagccgcagtcccacccgctgctggcaatctgaatgaggagccgcgctatttttacctccccggctgcaatcctttatatttacatgcaggaagcaaatatataagggattaagaaggagatgcgtggccttagtttatccagagcaggaagaggttggaataggagagggtatgtgaagtctggggtggtggaaaaggcaggtggacttcggctggttgttttctcccgatcatccctgtctctggcctggaaacccccgtactctctttcttctggcttatccgtgactgccggctccccctccaccgcccccatcttttgaggtaccacccgtcacctccgatgctgcttgggctgctgcatcactctgctgctttacccccttccccgccccccaacaaagcatgcgcagtgcgttccgggccaggcaacagcagcagcacagcatccagcaacagcatcagcacccgaagccccgctcgggcgcgctctcggggggcggggcgcacgcccgctccgcgcgtccccgcgccgctcgctcccgcgcgtccccgcgccgctcgctcccgcgcgccgcctcagcatcctcaggcccggcggcagcccccgcagtcgctgaagcggccgcgcccgccgggggagggagtagccgctggggaggctccaagttggcggagcggcgaggacccctggactcctctgcgtcccgccccgggagtggctgcgaggctaggcgagccgggaaagggggcgccgcccagccccgagccccgcgccccgtgccccgagcccggagccccctgcccgccgcggcaccatgcgcgccgagccggcgtgaccggctccgcccgcggccgccccgcagctagcccggcgctctcgccggccacacggagcggcgcccgggagctatgagccatgaagccgcccggcagcagctcgcggcagccgcccctggcgggctgcagccttgccggcgcttcctgcggcccccaacgcggccccgccggctcggtgcctgccagcgccccggcccgcacgccgccctgccgcctgcttctcgtccttctcctgctgcctccgctcgccgcctcgtcccggccccgcgcctggggggctgctgcgcccagcggtgggtatggccccgtgccctttgcgttggctttcccgcggggccctgcagaggaaagcgaagggcgcgcgggtccgtgtgctccgggcttgtccccggctcggcctttccttccctccctgcctgtctttccacccttctcgttcccaaacccccattcatcccagttcacttttggaagtccatttctgttgcattcgcgaaaaacccattccaattcttgttggttccactgggaggtgtttagtggatcctgggtccctcagcgatctctgtgcaacttgcggaggggcaaccagtggatgggaaatacagcgagggagcaagttgctacttgcgtggtggaaccttaatgtgaatgcggggaggatgtagtgataatagtggtaatgggctgtttcctcaaatttcgtatccggcgcattcagtgcggttggaattaaggtgggggaggcacacttcggggaccaaagaattaaggtgctgaagacatacttcatgcacgacctttggttctgatttctcaaagtgcttgtcattataatgaacaattaatataataccatcttctatatattgatgattggaagtcactgaaagcagaaagctggctttgtcaggaaaataaaaagaaattgggaagctgccagcatctgtatccctacatggc

SEQ ID NO: 14 - DNA, 3000 bp, Homo sapiens
tactgccgactttaggtctctctggatctcaggcccccttctctaagatgcatcctagaggaccaaaaatacactttatttgggcttcgcctgcttttgtggaagggtagtttactagaggatataatctcgtgttttaatttgctctctctcctaaaggaaatgtggagaaaaaaaaaaagcagaaattggaaataaccaatatttagtttatttcattcgattcttaggggaactggtgaggagcctaagatgattttcccttcctagagaaagaatccaaagtccagggaaatagcgacaggggagttcaagactgcccctgctagtccttccttggctactctccgctgcgatcgcaggatagctctcattagcaggagaatcgggcaagtgtgtggataagtagagagtgtgttgaacaacttgtaacgttttatgaaatacgcattgtcatggttccctaaaaggctttgcggaagccgtttgtctttactaatcaagtctttacttacacaaaagtagaagtagaagtagttttagaaaacatactaacaatcttctatccccttgaagaccagagtagcagaaaacaggtgatttgcattataaaattgcactcactttttcctcctttcagatttcacattacattagcccatttgtgttacggtgtataaaaaatggaacaggcgcctccactgcattgttctcctttaaaaatagatcacttacaccctaactttgttttccttaaattcgattcttaacaggagagctttctattatttcagatggagtgaggttgcacgactgggatggaagaaaggaatcccttaaatttgggggaatttctgttctctgttccaagaccattttacttggggtgtgggggtgggcgcggcggtcagggcagtggaacgcagtcgcggctgcgccatccctgcacttccaggcgcgcgggagggaccggcggggacgcgagctgcggactctggcgaactcgggggaggcagacagggggaggcggacacccagccggcaggcgtctcagcctccccgcagccggcgggcttttctcctgacagctccaggaaaggcagaccccttccccagccagccaggtaaggtaaagactgctgttgagcttgctgttactgagggcgcacagaccctggggagaccgaagcttgccactgcgggattctgtggggtaacctgggtctacggaagtttcctgaaagaggggagaagggtttgcatttttcctatggaggattcttctctctctagcatttcgtttgatgtattcaactggtagaagtgagatttcaacaggtagcagagagcgctcacgtggaggaggtttggggcgccgcggcgccacccccacccctcctcgggaccgcgcctatttctaaagttacacgtcgacgaactaacctatgctttaaattcctctttccagccccgtgagtccgcggcgacattgggccgtggggtggctgggaacggtcccctcctccggaaaaaccagagaacggcttggagagctgaaacgagcgtccgcgagcaggtccgtgcagaaccgggcttcaggaccgctgagctccgtagggcgtccttgggggacgccaggtcgccggctcctctgccctcgttgagatggacaacgcctcgttctcggagccctggcccgccaacgcatcgggcccggacccggcgctgagctgctccaacgcgtcgactctggcgccgctgccggcgccgctggcggtggctgtaccagttgtctacgcggtgatctgcgccgtgggtctggcgggcaactccgccgtgctgtacgtgttgctgcgggcgccccgcatgaagaccgtcaccaacctgttcatcctcaacctggccatcgccgacgagctcttcacgctggtgctgcccatcaacatcgccgacttcctgctgcggcagtggcccttcggggagctcatgtgcaagctcatcgtggctatcgaccagtacaacaccttctccagcctctacttcctcaccgtcatgagcgccgaccgctacctggtggtgttggccactgcggagtcgcgccgggtggccggccgcacctacagcgccgcgcgcgcggtgagcctggccgtgtgggggatcgtcacactcgtcgtgctgcccttcgcagtcttcgcccggctagacgacgagcagggccggcgccagtgcgtgctagtctttccgcagcccgaggccttctggtggcgcgcgagccgcctctacacgctcgtgctgggcttcgccatccccgtgtccaccatctgtgtcctctataccaccctgctgtgccggctgcatgccatgcggctggacagccacgccaaggccctggagcgcgccaagaagcgggtgaccttcctggtggtggcaatcctggcggtgtgcctcctctgctggacgccctaccacctgagcaccgtggtggcgctcaccaccgacctcccgcagacgccgctggtcatcgctatctcctacttcatcaccagcctgagctacgccaacagctgcctcaaccccttcctctacgccttcctggacgccagcttccgcaggaacctccgccagctgataacttgccgcgcggcagcctgactcccccagcgtccggctccgcaactgcccgccactcctggccagcgagggaggagccggcgccagagtgcgggaccagacaggccgcctaggcctcctggggaaaccgactcgcgccccatacccgacctagcagatcggaagcgctgcgactgtgcccgcaggttgaccttgccaagccctccaggtgatgcgcggccatgccgggtgaggagaactgaggctgagatcgccacactgagggctccctaaagccgaggtggaggaagaggagggtagaggaggagggcggtattgctgggaaccgccccctccctgccctgctccctgctgccccacccgagccct

SEQ ID NO: 15 - DNA, 3000 bp, Homo sapiens
gaatacattaaagtaggggcaacccttgagcccagacttctgccatgtgaagaccctttgaaaatcctgacaaacacaggtactgcgtaagtggtcagctaattaaagaggggaggtggagctgtcctttgtgtatccaataagtacccattatctcatttgagcatgaaaagaggccactgttattactttcaagaaggaaagtaagcaggatagctcatatttttagaaccattcctcaccaaatggaataattccggtgaaaagtgggagtgaggaagaaagaaaaaaaaaacttctaatcataatgtttgggaataagaaaggaagaagaaactcacgtcaaagccgactttctcctgcagctgtaaaataaactcttaagacccttcctgctgaaactctggagaggaaaactggagtggcgggtgggctttgcctgcagctcaactctccctcgcggcgcgggcgcggctgggttcagcacctcggaaagcgcccctcgcggcgccccgggattacgcatgctccttggggcccgccgccttggccgtgcaagtgccaccgtaactggtgagagccgctggcaacccacccggagttgacaaccgcggagagacgcagacacccactgacctccaggaagctgagcgtggtggatggaactctacgatctctttctctccaaggacggaaacctcatccaagcagtcccagaggaaacggataaaggtatttgaaagggagcgagcggccccaaatcgcacaattgagcggctgggggagttatgcgccagtgccccagtgaccgcgggacacggagaggggaagtctgcgttgtacataaggacctagggactccgagcttggcctgagaacccttggacgccgagtgcttgccttacgggctgcactcctcaactctgctccaaagcagccgctgagctcaactcctgcgtccagggcgttcgctgcgcgccaggacgcgcttagtacccagttcctgggctctctcttcagtagctgctttgaaagctcccacgcacgtcccgcaggctagcctggcaacaaaactggggtaaaccgtgttatcttaggtcttgtcccccagaacatgacctagaggtacctgcgcatgcagatggccgatgcagccacgatagccaccatgaataaggcagcaggcggggacaagctagcagaactcttcagtctggtcccggaccttctggaggcggccaacacgagtggtaacgcgtcgctgcagcttccggacttgtggtgggagctggggctggagttgccggacggcgcgccgccaggacatcccccgggcagcggcggggcagagagcgcggacacagaggcccgggtgcggattctcatcagcgtggtgtactgggtggtgtgcgccctggggttggcgggcaacctgctggttctctacctgatgaagagcatgcagggctggcgcaagtcctctatcaacctcttcgtcaccaacctggcgctgacggactttcagtttgtgctcaccctgcccttctgggcggtggagaacgctcttgacttcaaatggcccttcggcaaggccatgtgtaagatcgtgtccatggtgacgtccatgaacatgtacgccagcgtgttcttcctcactgccatgagtgtgacgcgctaccattcggtggcctcggctctgaagagccaccggacccgaggacacggccggggcgactgctgcggccggagcctgggggacagctgctgcttctcggccaaggcgctgtgtgtgtggatctgggctttggccgcgctggcctcgctgcccagtgccattttctccaccacggtcaaggtgatgggcgaggagctgtgcctggtgcgtttcccggacaagttgctgggccgcgacaggcagttctggctgggcctctaccactcgcagaaggtgctgctgggcttcgtgctgccgctgggcatcattatcttgtgctacctgctgctggtgcgcttcatcgccgaccgccgcgcggcggggaccaaaggaggggccgcggtagccggaggacgcccgaccggagccagcgcccggagactgtcgaaggtcaccaaatcagtgaccatcgttgtcctgtccttcttcctgtgttggctgcccaaccaggcgctcaccacctggagcatcctcatcaagttcaacgcggtgcccttcagccaggagtatttcctgtgccaggtatacgcgttccctgtgagcgtgtgcctagcgcactccaacagctgcctcaaccccgtcctctactgcctcgtgcgccgcgagttccgcaaggcgctcaagagcctgctgtggcgcatcgcgtctccttcgatcaccagcatgcgccccttcaccgccactaccaagccggagcacgaggatcaggggctgcaggccccggcgccgccccacgcggccgcggagccggacctgctctactacccacctggcgtcgtggtctacagcggggggcgctacgacctgctgcccagcagctctgcctactgacgcaggcctcaggcccagggcgcgccgtcggggcaaggtggccttccccgggcggtaaagaggtgaaaggatgaaggagggctggggggggccccatttaagaagtaggtgggaggaggatgggcagagcatggaggaggagcctgtggataggccgaggaccttctctggagaggagatgcttcgaaatcaggtggagagaggaaattggcaaagggatagagacgagccccacgggccagacagccaacctccgctccgcaccccacagcctctccttactcttcccacgctgagtagtgtgggggcgcccagaagcgaagacaagcagcaaaaatgtagagaaattggcacggggagcggggcttagccaaatgatgcacagacaattgtgcccgtttattccagcgacttctgcggagagggcagccgtcggcacaaacactcctttgcgt

SEQ ID NO: 16 - DNA, 2200 bp, Homo sapiens
gtcccccgattccctcacccatcatataacgtgtgtatttattatgtttcccgtttcctctgtctccgccagcagaatgtaaactccatgaggtcaggaatctccgagttatgttgcgccagtgtaatccaagagcccggaacagtgcctggcacacagcgggcatatggaagaacaaatgtgtgaaggtgtgaatgaatgaataattgaaagaataaatagtagttctcagcctcacagaacacgggtcacaacctcaaatgacctgctaccctgcccataaataacagagatgcaggagtaagtgctgggctgtgacctgtcaacatgctaagccgctcaaacaaaactgcccaacagcccgctggccgcctatttgcagcactgggccctgagccgcacattcccatttcgttgataaagaaactgaccagatagtttaagtggcctgctgcggaagacagagctggtgctgcaccggtcgctgcttccccagtccttttttggcctcctttctgacgcgacgcagaccccagttctggagagtctgtcactcgctccccgtggtgggagatcagaggcctggtgtccttgggagcggcgagcggtgctcggcgcaggatagaaagggagtgcgcgcccgagtcccccagatccctgggaacccgcgccaccctcccgcccctgcccatccccggccgcgctgtcagtctccattagcgctaacaggctccagacggagcgggccgggcgctgggttaatgcaatcggcgcgttacctggggcgcaggctacattaccagcccggcccccgccaggcacggccagaaccagtcagcccgcgccctgccggccgccccgcgcctccagctcttccccggccccgcccgaacgccacacggcggagcccagccccagcccgcgccctagagcctgccaaggcgccgccggtcgggggccggcagggcgcaaggcaccagggatcccctcgccgccggacacgtgagtgcgccctgagcgcgggacagggctaggtctgcctgggaggcccgggccgagacgcgccagcagagggctagcgagtttgtagtgcagtgacgttaagtgtccgagaaggctcctgtggctgttgaagtgtcgcggacctgagctggggagggggtcggcacgctgccctcagcctcggtgagttcaatcccagccatttggggcaggcgagagtgggtgaacgaggaaaagtgctgcagggtcttcagccgcccccagagggctgtcagaagtctccaactcttgagttccggcgtgccccaacctctgtttccaaatttttccagcggacgcgcgctcttttctgggaaccctgcgtccgctcagcgcgcgctcatcccagtgtctaaggcgctcccgggtggtcttgggagttgcaagtagggaggaacggccgggtaaccacctcttttccctttatccaagcagagcctcggcgtgcccccaggaccggtaaagttcctctcgccagccgcatccatgcttctggcgcggatgaacccgcaggtgcagcccgagaacaacggggcggacacgggtccagagcagccccttcgggcgcgcaaaactgcggagctgctggtggtgaaggagcgcaacggcgtccagtgcctgctggcgccccgcgacggcgacgcgcagccccgggagacctggggcaagaagatcgacttcctgctgtccgtagtcggcttcgcagtggacctggccaacgtgtggcgcttcccctacctctgctacaagaacggcggcggtgagcgtggggtcgggctgggaatttgaatctgggaggtccactgtctgcagcggtggctgggacaggagctggaatacacacggaagggaggcgaggagacaggggcaaatctggggcgcagaaagaactggacagggctaacgggaaaaaaaaaagattggagtcctctggaaggtcattttcccaggctctttgcagagtacctcgagctcattccagcggaagtgtcaggattgggcaccctggaagcaaaacagcagaagagtgaaatcgagtcatgaccctaaagtcatggtaggggtatggatggaaaggacagaatctggggtgccaggttgggtgggggagcctgaccttttgatggtctgctggaagggaggtggagattccaagagc

SEQ ID NO: 17 - DNA, 98 bp, Artificial Sequence
  Designed oligonucleotide, n=m5c
gttggccactgcggagtcgngcngggtggcnggccgcacctacagngccgngngngcggtgagcctggccgtgtgggggatcgtcacactcgtcgtgc

SEQ ID NO: 18 - DNA, 98 bp, Artificial Sequence
  Designed oligonucleotide, n=m5c
gttggccactgcggagtcgcgccgggtggcnggccgcacctacagngccgngngngcggtgagcctggccgtgtgggggatcgtcacactcgtcgtgc

SEQ ID NO: 19 - DNA, 98 bp, Artificial Sequence
  Designed oligonucleotide
gttggccactgcggagtcgcgccgggtggccggccgcacctacagcgccgcgcgcgcggtgagcctggccgtgtgggggatcgtcacactcgtcgtgc

SEQ ID NO: 20 - DNA, 14 bp, Artificial Sequence
  Designed biotinated oligonucleotide
gccacccggcgcga

SEQ ID NO: 21 - DNA, 19 bp, Artificial Sequence
  Designed oligonucleotide primer for PCR
gttggccactgcggagtcg

SEQ ID NO: 22 - DNA, 20 bp, Artificial Sequence
  Designed oligonucleotide primer for PCR
gcacgacgagtgtgacgatc

SEQ ID NO: 23 - DNA, 98 bp, Artificial Sequence; identical to SEQ ID NO: 19
  Designed oligonucleotide

SEQ ID NO: 24 - DNA, 18 bp, Artificial Sequence
  Designed oligonucleotide primer for PCR
ctcagcacccaggcggcc

SEQ ID NO: 25 - DNA, 20 bp, Artificial Sequence
  Designed oligonucleotide primer for PCR
ctggccaaactggagatcgc

SEQ ID NO: 26 - DNA, 386 bp, Artificial Sequence
  Designed oligonucleotide consist of objective DNA domain (Genbank Accession No.NT029419 25687390-256 87775 Homo sapiens)
ctcagcacccaggcggccgcgatcatgaggcgcgagcggcgcgcgggctgttgcagagtcttgagcgggtggcacaccgcgatgtagcggtcggctgtcatgactaccagcatgtaggccgacgcaaacatgccgaacacctgcaggtgcttcaccacgcggcacagccagtcggggccgcggaagcggtaggtgatgtcccagcacatttgcggcagcacctggaagaatgccacggccaggtcggccaggctgaggtgtcggatgaagaggtgcatgcgggacgtcttgcgcggcgtccggtgcagagccagcagtacgctgctgttgcccagcacggccaccgcgaaagtcaccgccagcacggcgatctccagtttggccag

SEQ ID NO: 27 - DNA, 386 bp, Artificial Sequence
  Designed oligonucleotide consist of objective DNA domain ( Genbank Accession No.NT029419 25687390-256 87775 Homo sapiens ) n=m5c
ctcagcacccaggnggcngngatcatgaggngngagnggngngngggctgttgcagagtcttgagngggtggcacacngngatgtagnggtnggctgtcatgactaccagcatgtaggcngangcaaacatgcngaacacctgcaggtgcttcaccangnggcacagccagtnggggcngnggaagnggtaggtgatgtcccagcacatttgnggcagcacctggaagaatgccanggccaggtnggccaggctgaggtgtnggatgaagaggtgcatgngggangtcttgngnggngtcnggtgcagagccagcagtangctgctgttgcccagcanggccacngngaaagtcacngccagcanggngatctccagtttggccag

SEQ ID NO: 28 - DNA, 386 bp, Artificial Sequence; identical to SEQ ID NO: 26
  Designed oligonucleotide consist of objective DNA domain ( Genbank Accession No.NT029419 25687390-25687775 Homo sapiens )

SEQ ID NO: 29 - DNA, 30 bp, Artificial Sequence
  Designed oligonucleotide for experiment
gccaccgcgaaagtcaccgccagcacggcg

SEQ ID NO: 30 - DNA, 30 bp, Artificial Sequence
  Designed oligonucleotide for experiment
gccagcagtacgctgctgttgcccagcacg

SEQ ID NO: 31 - DNA, 30 bp, Artificial Sequence
  Designed oligonucleotide for experiment
cgggacgtcttgcgcggcgtccggtgcaga

SEQ ID NO: 32 - DNA, 30 bp, Artificial Sequence
  Designed oligonucleotide for experiment
aggctgaggtgtcggatgaagaggtgcatg

SEQ ID NO: 33 - DNA, 30 bp, Artificial Sequence
  Designed oligonucleotide for experiment
acctggaagaatgccacggccaggtcggcc

SEQ ID NO: 34 - DNA, 30 bp, Artificial Sequence
  Designed oligonucleotide for experiment
taggtgatgtcccagcacatttgcggcagc

SEQ ID NO: 35 - DNA, 30 bp, Artificial Sequence
  Designed oligonucleotide for experiment
cggcacagccagtcggggccgcggaagcgg

SEQ ID NO: 36 - DNA, 30 bp, Artificial Sequence
  Designed oligonucleotide for experiment
atgccgaacacctgcaggtgcttcaccacg

SEQ ID NO: 37 - DNA, 30 bp, Artificial Sequence
  Designed oligonucleotide for experiment
atgactaccagcatgtaggccgacgcaaac

SEQ ID NO: 38 - DNA, 30 bp, Artificial Sequence
  Designed oligonucleotide for experiment
tggcacaccgcgatgtagcggtcggctgtc

SEQ ID NO: 39 - DNA, 30 bp, Artificial Sequence
  Designed oligonucleotide for experiment
cgcgcgggctgttgcagagtcttgagcggg

SEQ ID NO: 40 - DNA, 30 bp, Artificial Sequence
  Designed oligonucleotide for experiment
caggcggccgcgatcatgaggcgcgagcgg

SEQ ID NO: 41 - DNA, 12 bp, Artificial Sequence
  Designed oligonucleotide for experiment
tgcaccggacgc

SEQ ID NO: 42 - DNA, 19 bp, Artificial Sequence
  Designed oligonucleotide primer for PCR
tgagctccgtagggcgtcc

SEQ ID NO: 43 - DNA, 17 bp, Artificial Sequence
  Designed oligonucleotide primer for PCR
gcgccgggtccgggccc

SEQ ID NO: 44 - DNA, 121 bp, Artificial Sequence
  Designed oligonucleotide consist of objective DNA domain (Genbank Accession No.AC009800 76606-76726 Homo sapiens)
gcgccgggtccgggcccgatgcgttggcgggccagggctccgagaacgaggcgttgtccatctcaacgagggcagaggagccggcgacctggcgtcccccaaggacgccctacggagctca

SEQ ID NO: 45 - DNA, 121 bp, Artificial Sequence
  Designed oligonucleotide consist of objective DNA domain (Genbank Accession No.AC009800 76606-76726 Homo sapiens) n=m5c
gngcngggtcngggccngatgngttggngggccagggctcngagaangaggngttgtccatctcaangagggcagaggagcnggngacctggngtcccccaaggangccctanggagctca

SEQ ID NO: 46 - DNA, 121 bp, Artificial Sequence; identical to SEQ ID NO: 44
  Designed oligonucleotide consist of objective DNA domain (Genbank Accession No.AC009800 76606-76726 Homo sapiens)

SEQ ID NO: 47 - DNA, 30 bp, Artificial Sequence
  Designed oligonucleotide for experiment
gcgtcccccaaggacgccctacggagctca

SEQ ID NO: 48 - DNA, 30 bp, Artificial Sequence
  Designed oligonucleotide for experiment
ctcaacgagggcagaggagccggcgacctg

SEQ ID NO: 49 - DNA, 30 bp, Artificial Sequence
  Designed oligonucleotide for experiment
cgccgggtccgggcccgatgcgttggcggg

SEQ ID NO: 50 - DNA, 12 bp, Artificial Sequence
  Designed oligonucleotide for experiment
gtcgccggctcc

SEQ ID NO: 51 - DNA, 21 bp, Artificial Sequence
  Designed oligonucleotide primer for PCR
ggacctgtgtttgacgggtat

SEQ ID NO: 52 - DNA, 21 bp, Artificial Sequence
  Designed oligonucleotide primer for PCR
agtacagatctggcgttctcg

SEQ ID NO: 53 - DNA, 117 bp, Artificial Sequence
  Designed oligonucleotide consist of objective DNA domain ( Genbank Accession No.NC001139 384569-384685 Saccharomyces cereviciae chromosome VII )
ggacctgtgtttgacgggtataacactaagttgcgcaatttgctgtattgcgaaatccgcccggacgatatcactcttgagcgcatgtgccgtttccgagaacgccagatctgtact

SEQ ID NO: 54 - DNA, 117 bp, Artificial Sequence
  Designed oligonucleotide consist of objective DNA domain (Genbank Accession No.NC001139 384569-384685 Saccharomyces cereviciae chromosome VII ) n=m5c
ggacctgtgtttgangggtataacactaagttgngcaatttgctgtattgngaaatcngccnggangatatcactcttgagngcatgtgcngtttcngagaangccagatctgtact

SEQ ID NO: 55 - DNA, 117 bp, Artificial Sequence; identical to SEQ ID NO: 53
  Designed oligonucleotide consist of objective DNA domain (Genbank Accession No.NC001139 384569-384685 Saccharomyces cereviciae chromosome VII)

SEQ ID NO: 56 - DNA, 25 bp, Artificial Sequence
  Designed oligonucleotide for experiment
aacactaagttgcgcaatttgctgt

SEQ ID NO: 57 - DNA, 25 bp, Artificial Sequence
  Designed oligonucleotide for experiment
attgcgaaatccgcccggacgatat

SEQ ID NO: 58 - DNA, 25 bp, Artificial Sequence
  Designed oligonucleotide for experiment
cactcttgagcgcatgtgccgtttc

SEQ ID NO: 59 - DNA, 21 bp, Artificial Sequence
  Designed oligonucleotide for experiment
ggacctgtgtttgacgggtat

SEQ ID NO: 60 - DNA, 12 bp, Artificial Sequence
  Designed oligonucleotide for experiment
tcgtccgggcgg

SEQ ID NO: 61 - DNA, 244 bp, Artificial Sequence
  Designed oligonucleotide consist of objective DNA domain (Genbank Accession No.NC001139 384523-384766 Saccharomyces cereviciae chromosome VII)
taggaaatacattccgagggcgcccgcacaaggcctattattagagggacctgtgtttgacgggtataacactaagttgcgcaatttgctgtattgcgaaatccgcccggacgatatcactcttgagcgcatgtgccgtttccgagaacgccagatctgtactgcgatcgcacacgaggagacacagcgtcacgtgttttgccattttgtacgacaaatgaaccgcctggccacgcctctaatc

SEQ ID NO: 62 - DNA, 30 bp, Artificial Sequence
  Designed oligonucleotide for experiment
aatacattccgagggcgcccgcacaaggcc

SEQ ID NO: 63 - DNA, 21 bp, Artificial Sequence
  Designed oligonucleotide for experiment
gcgatcgcacacgaggagaca

SEQ ID NO: 64 - DNA, 30 bp, Artificial Sequence
  Designed oligonucleotide for experiment
agcgtcacgtgttttgccattttgtacgac

SEQ ID NO: 65 - DNA, 29 bp, Artificial Sequence
  Designed oligonucleotide for experiment
aaatgaaccgcctggccacgcctctaatc